SEQUENCE LISTING (804 sequences)

SEQ ID NO: 1 - protein, 10 aa, mycoplasma genitalium
  Sequence located in MG002 at 269-278 and may interact with Sequence 2 in this patent.
RLRIFSSFFE

SEQ ID NO: 2 - protein, 10 aa, mycoplasma genitalium
  Sequence located in MG081 at 82-91 and may interact with Sequence 1 in this patent.
LEKGAKNAKT

SEQ ID NO: 3 - protein, 10 aa, mycoplasma genitalium
  Sequence located in MG003 at 281-290 and may interact with Sequence 4 in this patent.
QSIFSFCNNI

SEQ ID NO: 4 - protein, 10 aa, mycoplasma genitalium
  Sequence located in MG305 at 516-525 and may interact with Sequence 3 in this patent.
LRNEGEGIVN

SEQ ID NO: 5 - protein, 10 aa, mycoplasma genitalium
  Sequence located in MG003 at 172-181 and may interact with Sequence 6 in this patent.
IVEFVPDFSV

SEQ ID NO: 6 - protein, 10 aa, mycoplasma genitalium
  Sequence located in MG122 at 396-405 and may interact with Sequence 5 in this patent.
NDLKNGVKRD

SEQ ID NO: 7 - protein, 10 aa, mycoplasma genitalium
  Sequence located in MG004 at 440-449 and may interact with Sequence 8 in this patent.
RSLSVLEVNK

SEQ ID NO: 8 - protein, 10 aa, mycoplasma genitalium
  Sequence located in MG051 at 196-205 and may interact with Sequence 7 in this patent.
FIDLKYGKGA

SEQ ID NO: 9 - protein, 10 aa, mycoplasma genitalium
  Sequence located in MG004 at 318-327 and may interact with Sequence 10 in this patent.
KRDTVPEVLL

SEQ ID NO: 10 - protein, 10 aa, mycoplasma genitalium
  Sequence located in MG036 at 137-146 and may interact with Sequence 9 in this patent.
LAIRNWFNQQ

SEQ ID NO: 11 - protein, 10 aa, mycoplasma genitalium
  Sequence located in MG004 at 229-238 and may interact with Sequence 12 in this patent.
FPTGANIIYT

SEQ ID NO: 12 - protein, 10 aa, mycoplasma genitalium
  Sequence located in MG231 at 688-697 and may interact with Sequence 11 in this patent.
KGCSSIYYVR

SEQ ID NO: 13 - protein, 10 aa, mycoplasma genitalium
  Sequence located in MG004 at 741-750 and may interact with Sequence 14 in this patent.
KRNAKGVKTL

SEQ ID NO: 14 - protein, 10 aa, mycoplasma genitalium
  Sequence located in MG414 at 433-442 and may interact with Sequence 13 in this patent.
LPISFSNLSK

SEQ ID NO: 15 - protein, 10 aa, mycoplasma genitalium
  Sequence located in MG007 at 166-175 and may interact with Sequence 16 in this patent.
EQIEAIENGS

SEQ ID NO: 16 - protein, 10 aa, mycoplasma genitalium
  Sequence located in MG241 at 66-75 and may interact with Sequence 15 in this patent.
LLNFSDFVSG

SEQ ID NO: 17 - protein, 10 aa, mycoplasma genitalium
  Sequence located in MG007 at 165-174 and may interact with Sequence 18 in this patent.
DEQIEAIENG

SEQ ID NO: 18 - protein, 10 aa, mycoplasma genitalium
  Sequence located in MG241 at 65-74 and may interact with Sequence 17 in this patent.
VLLNFSDFVS

SEQ ID NO: 19 - protein, 10 aa, mycoplasma genitalium
  Sequence located in MG008 at 399-408 and may interact with Sequence 20 in this patent.
KKYLFFDVLV

SEQ ID NO: 20 - protein, 10 aa, mycoplasma genitalium
  Sequence located in MG031 at 44-53 and may interact with Sequence 19 in this patent.
NKYIEEKVFL

SEQ ID NO: 21 - protein, 10 aa, mycoplasma genitalium
  Sequence located in MG009 at 168-177 and may interact with Sequence 22 in this patent.
LLSLKDLNID

SEQ ID NO: 22 - protein, 10 aa, mycoplasma genitalium
  Sequence located in MG028 at 154-163 and may interact with Sequence 21 in this patent.
IDIEILKAKK

SEQ ID NO: 23 - protein, 10 aa, mycoplasma genitalium
  Sequence located in MG014 at 7-16 and may interact with Sequence 24 in this patent.
EFNNKIRTAL

SEQ ID NO: 24 - protein, 10 aa, mycoplasma genitalium
  Sequence located in MG156 at 98-107 and may interact with Sequence 23 in this patent.
KRSSNLVIKL

SEQ ID NO: 25 - protein, 10 aa, mycoplasma genitalium
  Sequence located in MG015 at 184-193 and may interact with Sequence 26 in this patent.
SFAFLKKSKT

SEQ ID NO: 26 - protein, 10 aa, mycoplasma genitalium
  Sequence located in MG015 at 184-193 and may interact with Sequence 25 in this patent.
SFAFLKKSKT

SEQ ID NO: 27 - protein, 10 aa, mycoplasma genitalium
  Sequence located in MG015 at 148-157 and may interact with Sequence 28 in this patent.
AIQSLFNILT

SEQ ID NO: 28 - protein, 10 aa, mycoplasma genitalium
  Sequence located in MG247 at 88-97 and may interact with Sequence 27 in this patent.
GYLTEKVYQS

SEQ ID NO: 29 - protein, 10 aa, mycoplasma genitalium
  Sequence located in MG015 at 147-156 and may interact with Sequence 30 in this patent.
QAIQSLFNIL

SEQ ID NO: 30 - protein, 10 aa, mycoplasma genitalium
  Sequence located in MG025 at 18-27 and may interact with Sequence 29 in this patent.
QYIKKALDSL

SEQ ID NO: 31 - protein, 10 aa, mycoplasma genitalium
  Sequence located in MG015 at 148-157 and may interact with Sequence 32 in this patent.
AIQSLFNILT

SEQ ID NO: 32 - protein, 10 aa, mycoplasma genitalium
  Sequence located in MG025 at 17-26 and may interact with Sequence 31 in this patent.
CQYIKKALDS

SEQ ID NO: 33 - protein, 10 aa, mycoplasma genitalium
  Sequence located in MG015 at 149-158 and may interact with Sequence 34 in this patent.
IQSLFNILTT

SEQ ID NO: 34 - protein, 10 aa, mycoplasma genitalium
  Sequence located in MG025 at 16-25 and may interact with Sequence 33 in this patent.
CCQYIKKALD

SEQ ID NO: 35 - protein, 10 aa, mycoplasma genitalium
  Sequence located in MG015 at 294-303 and may interact with Sequence 36 in this patent.
LSYIASLRQI

SEQ ID NO: 36 - protein, 10 aa, mycoplasma genitalium
  Sequence located in MG075 at 390-399 and may interact with Sequence 35 in this patent.
EGINSAESLY

SEQ ID NO: 37 - protein, 10 aa, mycoplasma genitalium
  Sequence located in MG017 at 167-176 and may interact with Sequence 38 in this patent.
LTTVYPHRYT

SEQ ID NO: 38 - protein, 10 aa, mycoplasma genitalium
  Sequence located in MG066 at 19-28 and may interact with Sequence 37 in this patent.
KGGHVGMAIG

SEQ ID NO: 39 - protein, 10 aa, mycoplasma genitalium
  Sequence located in MG017 at 15-24 and may interact with Sequence 40 in this patent.
FLSLVKEVKK

SEQ ID NO: 40 - protein, 10 aa, mycoplasma genitalium
  Sequence located in MG266 at 671-680 and may interact with Sequence 39 in this patent.
FLNFLYKTKK

SEQ ID NO: 41 - protein, 10 aa, mycoplasma genitalium
  Sequence located in MG017 at 165-174 and may interact with Sequence 42 in this patent.
NTLTTVYPHR

SEQ ID NO: 42 - protein, 10 aa, mycoplasma genitalium
  Sequence located in MG429 at 218-227 and may interact with Sequence 41 in this patent.
TVGINGRKGI

SEQ ID NO: 43 - protein, 10 aa, mycoplasma genitalium
  Sequence located in MG032 at 191-200 and may interact with Sequence 44 in this patent.
LVSENDIIKH

SEQ ID NO: 44 - protein, 10 aa, mycoplasma genitalium
  Sequence located in MG119 at 99-108 and may interact with Sequence 43 in this patent.
VLDNIILGNE

SEQ ID NO: 45 - protein, 10 aa, mycoplasma genitalium
  Sequence located in MG040 at 397-406 and may interact with Sequence 46 in this patent.
ALDVAVESIL

SEQ ID NO: 46 - protein, 10 aa, mycoplasma genitalium
  Sequence located in MG286 at 86-95 and may interact with Sequence 45 in this patent.
GEINRYLRNE

SEQ ID NO: 47 - protein, 10 aa, mycoplasma genitalium
  Sequence located in MG040 at 366-375 and may interact with Sequence 48 in this patent.
AQELLDINID

SEQ ID NO: 48 - protein, 10 aa, mycoplasma genitalium
  Sequence located in MG239 at 680-689 and may interact with Sequence 47 in this patent.
SLFKKIDINI

SEQ ID NO: 49 - protein, 10 aa, mycoplasma genitalium
  Sequence located in MG040 at 435-444 and may interact with Sequence 50 in this patent.
GVSEAGYEFL

SEQ ID NO: 50 - protein, 10 aa, mycoplasma genitalium
  Sequence located in MG440 at 40-49 and may interact with Sequence 49 in this patent.
KELVTSLRDS

SEQ ID NO: 51 - protein, 10 aa, mycoplasma genitalium
  Sequence located in MG040 at 517-526 and may interact with Sequence 52 in this patent.
RNGSSDSNQQ

SEQ ID NO: 52 - protein, 10 aa, mycoplasma genitalium
  Sequence located in MG468 at 1096-1105 and may interact with Sequence 51 in this patent.
LLIGIAGTIP

SEQ ID NO: 53 - protein, 10 aa, mycoplasma genitalium
  Sequence located in MG048 at 134-143 and may interact with Sequence 54 in this patent.
VGLDIYRPAA

SEQ ID NO: 54 - protein, 10 aa, mycoplasma genitalium
  Sequence located in MG039 at 318-327 and may interact with Sequence 53 in this patent.
GSRAIDIETN

SEQ ID NO: 55 - protein, 10 aa, mycoplasma genitalium
  Sequence located in MG048 at 239-248 and may interact with Sequence 56 in this patent.
KLTGFIISKL

SEQ ID NO: 56 - protein, 10 aa, mycoplasma genitalium
  Sequence located in MG136 at 433-442 and may interact with Sequence 55 in this patent.
QLGNDETSEL

SEQ ID NO: 57 - protein, 10 aa, mycoplasma genitalium
  Sequence located in MG049 at 102-111 and may interact with Sequence 58 in this patent.
GDPLRAKWIA

SEQ ID NO: 58 - protein, 10 aa, mycoplasma genitalium
  Sequence located in MG049 at 102-111 and may interact with Sequence 57 in this patent.
GDPLRAKWIA

SEQ ID NO: 59 - protein, 10 aa, mycoplasma genitalium
  Sequence located in MG049 at 2-11 and may interact with Sequence 60 in this patent.
VCPTVLSLIA

SEQ ID NO: 60 - protein, 10 aa, mycoplasma genitalium
  Sequence located in MG154 at 56-65 and may interact with Sequence 59 in this patent.
HGGRHKRKYR

SEQ ID NO: 61 - protein, 10 aa, mycoplasma genitalium
  Sequence located in MG049 at 1-10 and may interact with Sequence 62 in this patent.
MVCPTVLSLI

SEQ ID NO: 62 - protein, 10 aa, mycoplasma genitalium
  Sequence located in MG154 at 55-64 and may interact with Sequence 61 in this patent.
HHGGRHKRKY

SEQ ID NO: 63 - protein, 10 aa, mycoplasma genitalium
  Sequence located in MG050 at 97-106 and may interact with Sequence 64 in this patent.
AEFKKRCACV

SEQ ID NO: 64 - protein, 10 aa, mycoplasma genitalium
  Sequence located in MG209 at 220-229 and may interact with Sequence 63 in this patent.
SLELLTGRTH

SEQ ID NO: 65 - protein, 10 aa, mycoplasma genitalium
  Sequence located in MG050 at 200-209 and may interact with Sequence 66 in this patent.
ASRIGTSNAV

SEQ ID NO: 66 - protein, 10 aa, mycoplasma genitalium
  Sequence located in MG348 at 169-178 and may interact with Sequence 65 in this patent.
RRSDASAVRY

SEQ ID NO: 67 - protein, 10 aa, mycoplasma genitalium
  Sequence located in MG054 at 119-128 and may interact with Sequence 68 in this patent.
GIVGSSGRGA

SEQ ID NO: 68 - protein, 10 aa, mycoplasma genitalium
  Sequence located in MG095 at 178-187 and may interact with Sequence 67 in this patent.
ANNTATSSTR

SEQ ID NO: 69 - protein, 10 aa, mycoplasma genitalium
  Sequence located in MG054 at 63-72 and may interact with Sequence 70 in this patent.
PRSLKNTTFN

SEQ ID NO: 70 - protein, 10 aa, mycoplasma genitalium
  Sequence located in MG163 at 80-89 and may interact with Sequence 69 in this patent.
RAGQLIGCKV

SEQ ID NO: 71 - protein, 10 aa, mycoplasma genitalium
  Sequence located in MG058 at 167-176 and may interact with Sequence 72 in this patent.
PSHNVAESIN

SEQ ID NO: 72 - protein, 10 aa, mycoplasma genitalium
  Sequence located in MG285 at 118-127 and may interact with Sequence 71 in this patent.
INRFSNVVAR

SEQ ID NO: 73 - protein, 10 aa, mycoplasma genitalium
  Sequence located in MG061 at 64-73 and may interact with Sequence 74 in this patent.
DGTSVELIKG

SEQ ID NO: 74 - protein, 10 aa, mycoplasma genitalium
  Sequence located in MG037 at 221-230 and may interact with Sequence 73 in this patent.
VARAHLQYFT

SEQ ID NO: 75 - protein, 10 aa, mycoplasma genitalium
  Sequence located in MG061 at 346-355 and may interact with Sequence 76 in this patent.
YEFKDYKKNE

SEQ ID NO: 76 - protein, 10 aa, mycoplasma genitalium
  Sequence located in MG225 at 391-400 and may interact with Sequence 75 in this patent.
FVFFIIFKFI

SEQ ID NO: 77 - protein, 10 aa, mycoplasma genitalium
  Sequence located in MG062 at 625-634 and may interact with Sequence 78 in this patent.
LLKTTLQGIE

SEQ ID NO: 78 - protein, 10 aa, mycoplasma genitalium
  Sequence located in MG279 at 99-108 and may interact with Sequence 77 in this patent.
LNALEGSLKK

SEQ ID NO: 79 - protein, 10 aa, mycoplasma genitalium
  Sequence located in MG062 at 626-635 and may interact with Sequence 80 in this patent.
LKTTLQGIEG

SEQ ID NO: 80 - protein, 10 aa, mycoplasma genitalium
  Sequence located in MG279 at 98-107 and may interact with Sequence 79 in this patent.
SLNALEGSLK

SEQ ID NO: 81 - protein, 10 aa, mycoplasma genitalium
  Sequence located in MG062 at 627-636 and may interact with Sequence 82 in this patent.
KTTLQGIEGA

SEQ ID NO: 82 - protein, 10 aa, mycoplasma genitalium
  Sequence located in MG279 at 97-106 and may interact with Sequence 81 in this patent.
SSLNALEGSL

SEQ ID NO: 83 - protein, 10 aa, mycoplasma genitalium
  Sequence located in MG062 at 46-55 and may interact with Sequence 84 in this patent.
FEALKKREEE

SEQ ID NO: 84 - protein, 10 aa, mycoplasma genitalium
  Sequence located in MG108 at 59-68 and may interact with Sequence 83 in this patent.
LFLSLFEGFE

SEQ ID NO: 85 - protein, 10 aa, mycoplasma genitalium
  Sequence located in MG065 at 104-113 and may interact with Sequence 86 in this patent.
ETVIASYNSF

SEQ ID NO: 86 - protein, 10 aa, mycoplasma genitalium
  Sequence located in MG065 at 104-113 and may interact with Sequence 85 in this patent.
ETVIASYNSF

SEQ ID NO: 87 - protein, 10 aa, mycoplasma genitalium
  Sequence located in MG065 at 303-312 and may interact with Sequence 88 in this patent.
LNDRKLTKFR

SEQ ID NO: 88 - protein, 10 aa, mycoplasma genitalium
  Sequence located in MG261 at 381-390 and may interact with Sequence 87 in this patent.
QIVTFQRFKT

SEQ ID NO: 89 - protein, 10 aa, mycoplasma genitalium
  Sequence located in MG065 at 302-311 and may interact with Sequence 90 in this patent.
CLNDRKLTKF

SEQ ID NO: 90 - protein, 10 aa, mycoplasma genitalium
  Sequence located in MG261 at 380-389 and may interact with Sequence 89 in this patent.
AQIVTFQRFK

SEQ ID NO: 91 - protein, 10 aa, mycoplasma genitalium
  Sequence located in MG065 at 393-402 and may interact with Sequence 92 in this patent.
FGDEPTGALD

SEQ ID NO: 92 - protein, 10 aa, mycoplasma genitalium
  Sequence located in MG301 at 99-108 and may interact with Sequence 91 in this patent.
IESTGRFVSE

SEQ ID NO: 93 - protein, 10 aa, mycoplasma genitalium
  Sequence located in MG066 at 615-624 and may interact with Sequence 94 in this patent.
GAFSFGESDD

SEQ ID NO: 94 - protein, 10 aa, mycoplasma genitalium
  Sequence located in MG072 at 104-113 and may interact with Sequence 93 in this patent.
TGEGKTLTIV

SEQ ID NO: 95 - protein, 10 aa, mycoplasma genitalium
  Sequence located in MG066 at 49-58 and may interact with Sequence 96 in this patent.
KWINRDRFVL

SEQ ID NO: 96 - protein, 10 aa, mycoplasma genitalium
  Sequence located in MG328 at 309-318 and may interact with Sequence 95 in this patent.
QNKPVAINPL

SEQ ID NO: 97 - protein, 10 aa, mycoplasma genitalium
  Sequence located in MG066 at 167-176 and may interact with Sequence 98 in this patent.
LYKLNKLIVL

SEQ ID NO: 98 - protein, 10 aa, mycoplasma genitalium
  Sequence located in MG341 at 238-247 and may interact with Sequence 97 in this patent.
EILKIFKNNE

SEQ ID NO: 99 - protein, 10 aa, mycoplasma genitalium
  Sequence located in MG066 at 594-603 and may interact with Sequence 100 in this patent.
EASSSYEWFC

SEQ ID NO: 100 - protein, 10 aa, mycoplasma genitalium
  Sequence located in MG450 at 114-123 and may interact with Sequence 99 in this patent.
TKPLVAGGRL

SEQ ID NO: 101 - protein, 10 aa, mycoplasma genitalium
  Sequence located in MG067 at 95-104 and may interact with Sequence 102 in this patent.
TIASSSIISS

SEQ ID NO: 102 - protein, 10 aa, mycoplasma genitalium
  Sequence located in MG274 at 239-248 and may interact with Sequence 101 in this patent.
AANYARGGNG

SEQ ID NO: 103 - protein, 10 aa, mycoplasma genitalium
  Sequence located in MG067 at 96-105 and may interact with Sequence 104 in this patent.
IASSSIISSV

SEQ ID NO: 104 - protein, 10 aa, mycoplasma genitalium
  Sequence located in MG274 at 238-247 and may interact with Sequence 103 in this patent.
DAANYARGGN

SEQ ID NO: 105 - protein, 10 aa, mycoplasma genitalium
  Sequence located in MG069 at 4-13 and may interact with Sequence 106 in this patent.
SLVKIRNKFK

SEQ ID NO: 106 - protein, 10 aa, mycoplasma genitalium
  Sequence located in MG072 at 722-731 and may interact with Sequence 105 in this patent.
TEHLDSVFKL

SEQ ID NO: 107 - protein, 10 aa, mycoplasma genitalium
  Sequence located in MG069 at 339-348 and may interact with Sequence 108 in this patent.
FLSWKELVDG

SEQ ID NO: 108 - protein, 10 aa, mycoplasma genitalium
  Sequence located in MG290 at 17-26 and may interact with Sequence 107 in this patent.
KKAPLLQNIS

SEQ ID NO: 109 - protein, 10 aa, mycoplasma genitalium
  Sequence located in MG069 at 479-488 and may interact with Sequence 110 in this patent.
APKGRRKEAS

SEQ ID NO: 110 - protein, 10 aa, mycoplasma genitalium
  Sequence located in MG098 at 80-89 and may interact with Sequence 109 in this patent.
GGLTAALFRG

SEQ ID NO: 111 - protein, 10 aa, mycoplasma genitalium
  Sequence located in MG069 at 317-326 and may interact with Sequence 112 in this patent.
ATTTGQDSTS

SEQ ID NO: 112 - protein, 10 aa, mycoplasma genitalium
  Sequence located in MG191 at 992-1001 and may interact with Sequence 111 in this patent.
SSSSTLVGSG

SEQ ID NO: 113 - protein, 10 aa, mycoplasma genitalium
  Sequence located in MG069 at 316-325 and may interact with Sequence 114 in this patent.
GATTTGQDST

SEQ ID NO: 114 - protein, 10 aa, mycoplasma genitalium
  Sequence located in MG191 at 991-1000 and may interact with Sequence 113 in this patent.
SSSSSTLVGS

SEQ ID NO: 115 - protein, 10 aa, mycoplasma genitalium
  Sequence located in MG069 at 281-290 and may interact with Sequence 116 in this patent.
GSDYVTRVIG

SEQ ID NO: 116 - protein, 10 aa, mycoplasma genitalium
  Sequence located in MG346 at 16-25 and may interact with Sequence 115 in this patent.
PNNTGNIVRS

SEQ ID NO: 117 - protein, 10 aa, mycoplasma genitalium
  Sequence located in MG070 at 43-52 and may interact with Sequence 118 in this patent.
RKHNNDLLKL

SEQ ID NO: 118 - protein, 10 aa, mycoplasma genitalium
  Sequence located in MG223 at 72-81 and may interact with Sequence 117 in this patent.
KLEKVVVVLA

SEQ ID NO: 119 - protein, 10 aa, mycoplasma genitalium
  Sequence located in MG071 at 403-412 and may interact with Sequence 120 in this patent.
EKAFDSIRKL

SEQ ID NO: 120 - protein, 10 aa, mycoplasma genitalium
  Sequence located in MG181 at 259-268 and may interact with Sequence 119 in this patent.
ELAYGIERLL

SEQ ID NO: 121 - protein, 10 aa, mycoplasma genitalium
  Sequence located in MG071 at 217-226 and may interact with Sequence 122 in this patent.
GKIAKTVDDS

SEQ ID NO: 122 - protein, 10 aa, mycoplasma genitalium
  Sequence located in MG241 at 485-494 and may interact with Sequence 121 in this patent.
TVINRLRYLS

SEQ ID NO: 123 - protein, 10 aa, mycoplasma genitalium
  Sequence located in MG071 at 174-183 and may interact with Sequence 124 in this patent.
DKTIDSNEKA

SEQ ID NO: 124 - protein, 10 aa, mycoplasma genitalium
  Sequence located in MG334 at 659-668 and may interact with Sequence 123 in this patent.
VLSNIAILLS

SEQ ID NO: 125 - protein, 10 aa, mycoplasma genitalium
  Sequence located in MG072 at 326-335 and may interact with Sequence 126 in this patent.
EGRSYSNGLQ

SEQ ID NO: 126 - protein, 10 aa, mycoplasma genitalium
  Sequence located in MG195 at 485-494 and may interact with Sequence 125 in this patent.
LKPIGITSSF

SEQ ID NO: 127 - protein, 10 aa, mycoplasma genitalium
  Sequence located in MG073 at 365-374 and may interact with Sequence 128 in this patent.
IDYGFRLPSA

SEQ ID NO: 128 - protein, 10 aa, mycoplasma genitalium
  Sequence located in MG140 at 316-325 and may interact with Sequence 127 in this patent.
GTGKSETIVN

SEQ ID NO: 129 - protein, 10 aa, mycoplasma genitalium
  Sequence located in MG073 at 236-245 and may interact with Sequence 130 in this patent.
KILNQLFEIK

SEQ ID NO: 130 - protein, 10 aa, mycoplasma genitalium
  Sequence located in MG462 at 295-304 and may interact with Sequence 129 in this patent.
FNLKQLIENF

SEQ ID NO: 131 - protein, 10 aa, mycoplasma genitalium
  Sequence located in MG074 at 3-12 and may interact with Sequence 132 in this patent.
QFIKLSLLVF

SEQ ID NO: 132 - protein, 10 aa, mycoplasma genitalium
  Sequence located in MG239 at 223-232 and may interact with Sequence 131 in this patent.
KNKETEFDKL

SEQ ID NO: 133 - protein, 10 aa, mycoplasma genitalium
  Sequence located in MG077 at 109-118 and may interact with Sequence 134 in this patent.
LIGSLLGIFL

SEQ ID NO: 134 - protein, 10 aa, mycoplasma genitalium
  Sequence located in MG037 at 187-196 and may interact with Sequence 133 in this patent.
EKYAKETADE

SEQ ID NO: 135 - protein, 10 aa, mycoplasma genitalium
  Sequence located in MG077 at 369-378 and may interact with Sequence 136 in this patent.
YKTKTVQLTT

SEQ ID NO: 136 - protein, 10 aa, mycoplasma genitalium
  Sequence located in MG147 at 152-161 and may interact with Sequence 135 in this patent.
RSELYRLCLV

SEQ ID NO: 137 - protein, 10 aa, mycoplasma genitalium
  Sequence located in MG077 at 370-379 and may interact with Sequence 138 in this patent.
KTKTVQLTTE

SEQ ID NO: 138 - protein, 10 aa, mycoplasma genitalium
  Sequence located in MG147 at 151-160 and may interact with Sequence 137 in this patent.
FRSELYRLCL

SEQ ID NO: 139 - protein, 10 aa, mycoplasma genitalium
  Sequence located in MG079 at 250-259 and may interact with Sequence 140 in this patent.
LIKQLRDLLN

SEQ ID NO: 140 - protein, 10 aa, mycoplasma genitalium
  Sequence located in MG334 at 606-615 and may interact with Sequence 139 in this patent.
IQKITKLLDK

SEQ ID NO: 141 - protein, 10 aa, mycoplasma genitalium
  Sequence located in MG079 at 59-68 and may interact with Sequence 142 in this patent.
ESGSGKSVSV

SEQ ID NO: 142 - protein, 10 aa, mycoplasma genitalium
  Sequence located in MG459 at 59-68 and may interact with Sequence 141 in this patent.
FGATALGDGH

SEQ ID NO: 143 - protein, 10 aa, mycoplasma genitalium
  Sequence located in MG080 at 684-693 and may interact with Sequence 144 in this patent.
EFSGGQRQRI

SEQ ID NO: 144 - protein, 10 aa, mycoplasma genitalium
  Sequence located in MG261 at 235-244 and may interact with Sequence 143 in this patent.
LKAASLSLTD

SEQ ID NO: 145 - protein, 10 aa, mycoplasma genitalium
  Sequence located in MG080 at 731-740 and may interact with Sequence 146 in this patent.
AKKHNLTVLF

SEQ ID NO: 146 - protein, 10 aa, mycoplasma genitalium
  Sequence located in MG300 at 55-64 and may interact with Sequence 145 in this patent.
KKNCKIVLLS

SEQ ID NO: 147 - protein, 10 aa, mycoplasma genitalium
  Sequence located in MG244 at 392-401 and may interact with Sequence 148 in this patent.
QLVKLQRVLI

SEQ ID NO: 148 - protein, 10 aa, mycoplasma genitalium
  Sequence located in MG400 at 253-262 and may interact with Sequence 147 in this patent.
DKYTLQFNKL

SEQ ID NO: 149 - protein, 10 aa, mycoplasma genitalium
  Sequence located in MG248 at 21-30 and may interact with Sequence 150 in this patent.
YDIGCDHSYL

SEQ ID NO: 150 - protein, 10 aa, mycoplasma genitalium
  Sequence located in MG335 at 107-116 and may interact with Sequence 149 in this patent.
IVDSGVVTVQ

SEQ ID NO: 151 - protein, 10 aa, mycoplasma genitalium
  Sequence located in MG249 at 204-213 and may interact with Sequence 152 in this patent.
DKVDDNVRFF

SEQ ID NO: 152 - protein, 10 aa, mycoplasma genitalium
  Sequence located in MG415 at 229-238 and may interact with Sequence 151 in this patent.
KKTDIIVYLI

SEQ ID NO: 153 - protein, 10 aa, mycoplasma genitalium
  Sequence located in MG256 at 202-211 and may interact with Sequence 154 in this patent.
PVLNFQRLYV

SEQ ID NO: 154 - protein, 10 aa, mycoplasma genitalium
  Sequence located in MG018 at 19-28 and may interact with Sequence 153 in this patent.
DIKALKIEDR

SEQ ID NO: 155 - protein, 10 aa, mycoplasma genitalium
  Sequence located in MG258 at 325-334 and may interact with Sequence 156 in this patent.
LTINKLNTVI

SEQ ID NO: 156 - protein, 10 aa, mycoplasma genitalium
  Sequence located in MG085 at 14-23 and may interact with Sequence 155 in this patent.
NDCIQLIDGK

SEQ ID NO: 157 - protein, 10 aa, mycoplasma genitalium
  Sequence located in MG262 at 61-70 and may interact with Sequence 158 in this patent.
FDTKAPTFRD

SEQ ID NO: 158 - protein, 10 aa, mycoplasma genitalium
  Sequence located in MG266 at 178-187 and may interact with Sequence 157 in this patent.
VSERGSFSVE

SEQ ID NO: 159 - protein, 10 aa, mycoplasma genitalium
  Sequence located in MG270 at 219-228 and may interact with Sequence 160 in this patent.
VLTKDALAKV

SEQ ID NO: 160 - protein, 10 aa, mycoplasma genitalium
  Sequence located in MG445 at 158-167 and may interact with Sequence 159 in this patent.
DQSLICESFN

SEQ ID NO: 161 - protein, 10 aa, mycoplasma genitalium
  Sequence located in MG270 at 218-227 and may interact with Sequence 162 in this patent.
IVLTKDALAK

SEQ ID NO: 162 - protein, 10 aa, mycoplasma genitalium
  Sequence located in MG445 at 157-166 and may interact with Sequence 161 in this patent.
NDQSLICESF

SEQ ID NO: 163 - protein, 10 aa, mycoplasma genitalium
  Sequence located in MG271 at 243-252 and may interact with Sequence 164 in this patent.
LFYTVNGVEQ

SEQ ID NO: 164 - protein, 10 aa, mycoplasma genitalium
  Sequence located in MG271 at 243-252 and may interact with Sequence 163 in this patent.
LFYTVNGVEQ

SEQ ID NO: 165 - protein, 10 aa, mycoplasma genitalium
  Sequence located in MG271 at 253-262 and may interact with Sequence 166 in this patent.
SVIGDKILVS

SEQ ID NO: 166 - protein, 10 aa, mycoplasma genitalium
  Sequence located in MG192 at 438-447 and may interact with Sequence 165 in this patent.
GYKDLVTNDT

SEQ ID NO: 167 - protein, 10 aa, mycoplasma genitalium
  Sequence located in MG272 at 187-196 and may interact with Sequence 168 in this patent.
NATKLKQYRE

SEQ ID NO: 168 - protein, 10 aa, mycoplasma genitalium
  Sequence located in MG322 at 102-111 and may interact with Sequence 167 in this patent.
LAVLLQLGGI

SEQ ID NO: 169 - protein, 10 aa, mycoplasma genitalium
  Sequence located in MG276 at 85-94 and may interact with Sequence 170 in this patent.
ANKLPGQLIS

SEQ ID NO: 170 - protein, 10 aa, mycoplasma genitalium
  Sequence located in MG276 at 85-94 and may interact with Sequence 169 in this patent.
ANKLPGQLIS

SEQ ID NO: 171 - protein, 10 aa, mycoplasma genitalium
  Sequence located in MG277 at 160-169 and may interact with Sequence 172 in this patent.
QVRRNAISSV

SEQ ID NO: 172 - protein, 10 aa, mycoplasma genitalium
  Sequence located in MG156 at 51-60 and may interact with Sequence 171 in this patent.
LNSAISNATN

SEQ ID NO: 173 - protein, 10 aa, mycoplasma genitalium
  Sequence located in MG277 at 551-560 and may interact with Sequence 174 in this patent.
SVVSSLVIFS

SEQ ID NO: 174 - protein, 10 aa, mycoplasma genitalium
  Sequence located in MG211 at 23-32 and may interact with Sequence 173 in this patent.
AKDDKGGYDA

SEQ ID NO: 175 - protein, 10 aa, mycoplasma genitalium
  Sequence located in MG277 at 208-217 and may interact with Sequence 176 in this patent.
VNSALTFEYK

SEQ ID NO: 176 - protein, 10 aa, mycoplasma genitalium
  Sequence located in MG239 at 640-649 and may interact with Sequence 175 in this patent.
HVAGKGELIL

SEQ ID NO: 177 - protein, 10 aa, mycoplasma genitalium
  Sequence located in MG277 at 207-216 and may interact with Sequence 178 in this patent.
SVNSALTFEY

SEQ ID NO: 178 - protein, 10 aa, mycoplasma genitalium
  Sequence located in MG239 at 639-648 and may interact with Sequence 177 in this patent.
THVAGKGELI

SEQ ID NO: 179 - protein, 10 aa, mycoplasma genitalium
  Sequence located in MG277 at 533-542 and may interact with Sequence 180 in this patent.
LTLRYKLLGL

SEQ ID NO: 180 - protein, 10 aa, mycoplasma genitalium
  Sequence located in MG386 at 1055-1064 and may interact with Sequence 179 in this patent.
ESEAVFEKPQ

SEQ ID NO: 181 - protein, 10 aa, mycoplasma genitalium
  Sequence located in MG277 at 631-640 and may interact with Sequence 182 in this patent.
VIYLSNYQVQ

SEQ ID NO: 182 - protein, 10 aa, mycoplasma genitalium
  Sequence located in MG401 at 57-66 and may interact with Sequence 181 in this patent.
NNIQGIVLNL

SEQ ID NO: 183 - protein, 10 aa, mycoplasma genitalium
  Sequence located in MG278 at 172-181 and may interact with Sequence 184 in this patent.
TLELYAKIAG

SEQ ID NO: 184 - protein, 10 aa, mycoplasma genitalium
  Sequence located in MG101 at 37-46 and may interact with Sequence 183 in this patent.
RQLEIRFNSS

SEQ ID NO: 185 - protein, 10 aa, mycoplasma genitalium
  Sequence located in MG279 at 12-21 and may interact with Sequence 186 in this patent.
LIILVGILLL

SEQ ID NO: 186 - protein, 10 aa, mycoplasma genitalium
  Sequence located in MG217 at 135-144 and may interact with Sequence 185 in this patent.
QYYQDPNQQQ

SEQ ID NO: 187 - protein, 10 aa, mycoplasma genitalium
  Sequence located in MG279 at 12-21 and may interact with Sequence 188 in this patent.
LIILVGILLL

SEQ ID NO: 188 - protein, 10 aa, mycoplasma genitalium
  Sequence located in MG217 at 116-125 and may interact with Sequence 187 in this patent.
QYYQDPNQQQ

SEQ ID NO: 189 - protein, 10 aa, mycoplasma genitalium
  Sequence located in MG279 at 12-21 and may interact with Sequence 190 in this patent.
LIILVGILLL

SEQ ID NO: 190 - protein, 10 aa, mycoplasma genitalium
  Sequence located in MG217 at 97-106 and may interact with Sequence 189 in this patent.
QYYQDPNQQQ

SEQ ID NO: 191 - protein, 10 aa, mycoplasma genitalium
  Sequence located in MG279 at 12-21 and may interact with Sequence 192 in this patent.
LIILVGILLL

SEQ ID NO: 192 - protein, 10 aa, mycoplasma genitalium
  Sequence located in MG217 at 77-86 and may interact with Sequence 191 in this patent.
QYYQDPNQQQ

SEQ ID NO: 193 - protein, 10 aa, mycoplasma genitalium
  Sequence located in MG279 at 12-21 and may interact with Sequence 194 in this patent.
LIILVGILLL

SEQ ID NO: 194 - protein, 10 aa, mycoplasma genitalium
  Sequence located in MG217 at 57-66 and may interact with Sequence 193 in this patent.
QYYQDPNQQQ

SEQ ID NO: 195 - protein, 10 aa, mycoplasma genitalium
  Sequence located in MG279 at 12-21 and may interact with Sequence 196 in this patent.
LIILVGILLL

SEQ ID NO: 196 - protein, 10 aa, mycoplasma genitalium
  Sequence located in MG217 at 25-34 and may interact with Sequence 195 in this patent.
QYYQDPNQQQ

SEQ ID NO: 197 - protein, 10 aa, mycoplasma genitalium
  Sequence located in MG280 at 102-111 and may interact with Sequence 198 in this patent.
ATKKDLENSK

SEQ ID NO: 198 - protein, 10 aa, mycoplasma genitalium
  Sequence located in MG043 at 105-114 and may interact with Sequence 197 in this patent.
SSFLIKLIGL

SEQ ID NO: 199 - protein, 10 aa, mycoplasma genitalium
  Sequence located in MG281 at 270-279 and may interact with Sequence 200 in this patent.
GLSGNGSQLN

SEQ ID NO: 200 - protein, 10 aa, mycoplasma genitalium
  Sequence located in MG095 at 34-43 and may interact with Sequence 199 in this patent.
AQTTISTLKI

SEQ ID NO: 201 - protein, 10 aa, mycoplasma genitalium
  Sequence located in MG281 at 269-278 and may interact with Sequence 202 in this patent.
RGLSGNGSQL

SEQ ID NO: 202 - protein, 10 aa, mycoplasma genitalium
  Sequence located in MG095 at 33-42 and may interact with Sequence 201 in this patent.
PAQTTISTLK

SEQ ID NO: 203 - protein, 10 aa, mycoplasma genitalium
  Sequence located in MG281 at 203-212 and may interact with Sequence 204 in this patent.
TKATTVSTSN

SEQ ID NO: 204 - protein, 10 aa, mycoplasma genitalium
  Sequence located in MG169 at 20-29 and may interact with Sequence 203 in this patent.
VGRGHGSGLG

SEQ ID NO: 205 - protein, 10 aa, mycoplasma genitalium
  Sequence located in MG281 at 204-213 and may interact with Sequence 206 in this patent.
KATTVSTSNN

SEQ ID NO: 206 - protein, 10 aa, mycoplasma genitalium
  Sequence located in MG169 at 19-28 and may interact with Sequence 205 in this patent.
VVGRGHGSGL

SEQ ID NO: 207 - protein, 10 aa, mycoplasma genitalium
  Sequence located in MG281 at 205-214 and may interact with Sequence 208 in this patent.
ATTVSTSNNL

SEQ ID NO: 208 - protein, 10 aa, mycoplasma genitalium
  Sequence located in MG169 at 18-27 and may interact with Sequence 207 in this patent.
KVVGRGHGSG

SEQ ID NO: 209 - protein, 10 aa, mycoplasma genitalium
  Sequence located in MG281 at 206-215 and may interact with Sequence 210 in this patent.
TTVSTSNNLT

SEQ ID NO: 210 - protein, 10 aa, mycoplasma genitalium
  Sequence located in MG169 at 17-26 and may interact with Sequence 209 in this patent.
SKVVGRGHGS

SEQ ID NO: 211 - protein, 10 aa, mycoplasma genitalium
  Sequence located in MG281 at 429-438 and may interact with Sequence 212 in this patent.
YREGDNTYYR

SEQ ID NO: 212 - protein, 10 aa, mycoplasma genitalium
  Sequence located in MG314 at 388-397 and may interact with Sequence 211 in this patent.
VPFSIISVIA

SEQ ID NO: 213 - protein, 10 aa, mycoplasma genitalium
  Sequence located in MG282 at 146-155 and may interact with Sequence 214 in this patent.
GIEVPYRVKI

SEQ ID NO: 214 - protein, 10 aa, mycoplasma genitalium
  Sequence located in MG210.1 at 23-32 and may interact with Sequence 213 in this patent.
TDFDWVTYLN

SEQ ID NO: 215 - protein, 10 aa, mycoplasma genitalium
  Sequence located in MG282 at 20-29 and may interact with Sequence 216 in this patent.
LENLIQVKRP

SEQ ID NO: 216 - protein, 10 aa, mycoplasma genitalium
  Sequence located in MG379 at 357-366 and may interact with Sequence 215 in this patent.
KLIENLYFAG

SEQ ID NO: 217 - protein, 10 aa, mycoplasma genitalium
  Sequence located in MG289 at 2-11 and may interact with Sequence 218 in this patent.
LFKKFTWVIP

SEQ ID NO: 218 - protein, 10 aa, mycoplasma genitalium
  Sequence located in MG023 at 257-266 and may interact with Sequence 217 in this patent.
GYDPRKLLKE

SEQ ID NO: 219 - protein, 10 aa, mycoplasma genitalium
  Sequence located in MG289 at 250-259 and may interact with Sequence 220 in this patent.
AVVRGREIGI

SEQ ID NO: 220 - protein, 10 aa, mycoplasma genitalium
  Sequence located in MG191 at 853-862 and may interact with Sequence 219 in this patent.
SNNSTPFDPN

SEQ ID NO: 221 - protein, 10 aa, mycoplasma genitalium
  Sequence located in MG290 at 222-231 and may interact with Sequence 222 in this patent.
NHALVLDRLT

SEQ ID NO: 222 - protein, 10 aa, mycoplasma genitalium
  Sequence located in MG171 at 41-50 and may interact with Sequence 221 in this patent.
REAIKNQSVI

SEQ ID NO: 223 - protein, 10 aa, mycoplasma genitalium
  Sequence located in MG290 at 223-232 and may interact with Sequence 224 in this patent.
HALVLDRLTE

SEQ ID NO: 224 - protein, 10 aa, mycoplasma genitalium
  Sequence located in MG171 at 40-49 and may interact with Sequence 223 in this patent.
FREAIKNQSV

SEQ ID NO: 225 - protein, 10 aa, mycoplasma genitalium
  Sequence located in MG290 at 224-233 and may interact with Sequence 226 in this patent.
ALVLDRLTEK

SEQ ID NO: 226 - protein, 10 aa, mycoplasma genitalium
  Sequence located in MG171 at 39-48 and may interact with Sequence 225 in this patent.
LFREAIKNQS

SEQ ID NO: 227 - protein, 10 aa, mycoplasma genitalium
  Sequence located in MG290 at 121-130 and may interact with Sequence 228 in this patent.
KWIKDKILAI

SEQ ID NO: 228 - protein, 10 aa, mycoplasma genitalium
  Sequence located in MG366 at 485-494 and may interact with Sequence 227 in this patent.
NCENLIFDPL

SEQ ID NO: 229 - protein, 10 aa, mycoplasma genitalium
  Sequence located in MG291 at 287-296 and may interact with Sequence 230 in this patent.
RIIFILFIVV

SEQ ID NO: 230 - protein, 10 aa, mycoplasma genitalium
  Sequence located in MG328 at 403-412 and may interact with Sequence 229 in this patent.
SNDENEKYDD

SEQ ID NO: 231 - protein, 10 aa, mycoplasma genitalium
  Sequence located in MG297 at 258-267 and may interact with Sequence 232 in this patent.
TLLVLDGTVG

SEQ ID NO: 232 - protein, 10 aa, mycoplasma genitalium
  Sequence located in MG341 at 802-811 and may interact with Sequence 231 in this patent.
GQEHKIASDS

SEQ ID NO: 233 - protein, 10 aa, mycoplasma genitalium
  Sequence located in MG297 at 294-303 and may interact with Sequence 234 in this patent.
SAKGGIILAI

SEQ ID NO: 234 - protein, 10 aa, mycoplasma genitalium
  Sequence located in MG393 at 29-38 and may interact with Sequence 233 in this patent.
TSLASNDKSD

SEQ ID NO: 235 - protein, 10 aa, mycoplasma genitalium
  Sequence located in MG297 at 163-172 and may interact with Sequence 236 in this patent.
QNKRVLLVAG

SEQ ID NO: 236 - protein, 10 aa, mycoplasma genitalium
  Sequence located in MG462 at 447-456 and may interact with Sequence 235 in this patent.
LIFTNKEHGP

SEQ ID NO: 237 - protein, 10 aa, mycoplasma genitalium
  Sequence located in MG298 at 727-736 and may interact with Sequence 238 in this patent.
IQNITEKINS

SEQ ID NO: 238 - protein, 10 aa, mycoplasma genitalium
  Sequence located in MG122 at 533-542 and may interact with Sequence 237 in this patent.
NLVNRFFYIT

SEQ ID NO: 239 - protein, 10 aa, mycoplasma genitalium
  Sequence located in MG298 at 495-504 and may interact with Sequence 240 in this patent.
LDGFLGVCSD

SEQ ID NO: 240 - protein, 10 aa, mycoplasma genitalium
  Sequence located in MG374 at 112-121 and may interact with Sequence 239 in this patent.
KISEEPNTTI

SEQ ID NO: 241 - protein, 10 aa, mycoplasma genitalium
  Sequence located in MG305 at 279-288 and may interact with Sequence 242 in this patent.
KLTRAKFEEL

SEQ ID NO: 242 - protein, 10 aa, mycoplasma genitalium
  Sequence located in MG414 at 825-834 and may interact with Sequence 241 in this patent.
LKSSSFKFLE

SEQ ID NO: 243 - protein, 10 aa, mycoplasma genitalium
  Sequence located in MG310 at 258-267 and may interact with Sequence 244 in this patent.
SLVNDFFQLI

SEQ ID NO: 244 - protein, 10 aa, mycoplasma genitalium
  Sequence located in MG130 at 402-411 and may interact with Sequence 243 in this patent.
DELKKIVDKT

SEQ ID NO: 245 - protein, 10 aa, mycoplasma genitalium
  Sequence located in MG310 at 46-55 and may interact with Sequence 246 in this patent.
YSFYAINYPG

SEQ ID NO: 246 - protein, 10 aa, mycoplasma genitalium
  Sequence located in MG421 at 226-235 and may interact with Sequence 245 in this patent.
SRIVDSIETI

SEQ ID NO: 247 - protein, 10 aa, mycoplasma genitalium
  Sequence located in MG311 at 78-87 and may interact with Sequence 248 in this patent.
VLKQKGNLTV

SEQ ID NO: 248 - protein, 10 aa, mycoplasma genitalium
  Sequence located in MG462 at 356-365 and may interact with Sequence 247 in this patent.
NREISLLFKN

SEQ ID NO: 249 - protein, 10 aa, mycoplasma genitalium
  Sequence located in MG001 at 247-256 and may interact with Sequence 250 in this patent.
FEPETKIVVQ

SEQ ID NO: 250 - protein, 10 aa, mycoplasma genitalium
  Sequence located in MG428 at 50-59 and may interact with Sequence 249 in this patent.
EFGLSFDNYL

SEQ ID NO: 251 - protein, 10 aa, mycoplasma genitalium
  Sequence located in MG010 at 77-86 and may interact with Sequence 252 in this patent.
RKVSYKKEFL

SEQ ID NO: 252 - protein, 10 aa, mycoplasma genitalium
  Sequence located in MG414 at 538-547 and may interact with Sequence 251 in this patent.
EELFFIRNLP

SEQ ID NO: 253 - protein, 10 aa, mycoplasma genitalium
  Sequence located in MG010 at 37-46 and may interact with Sequence 254 in this patent.
RGFNLQDFLK

SEQ ID NO: 254 - protein, 10 aa, mycoplasma genitalium
  Sequence located in MG433 at 2-11 and may interact with Sequence 253 in this patent.
ATKIELIKEL

SEQ ID NO: 255 - protein, 10 aa, mycoplasma genitalium
  Sequence located in MG020 at 282-291 and may interact with Sequence 256 in this patent.
YVTNNAGHSG

SEQ ID NO: 256 - protein, 10 aa, mycoplasma genitalium
  Sequence located in MG294 at 60-69 and may interact with Sequence 255 in this patent.
ATVTSIIGYV

SEQ ID NO: 257 - protein, 10 aa, mycoplasma genitalium
  Sequence located in MG024 at 351-360 and may interact with Sequence 258 in this patent.
NYLIKDGDVC

SEQ ID NO: 258 - protein, 10 aa, mycoplasma genitalium
  Sequence located in MG180 at 267-276 and may interact with Sequence 257 in this patent.
VIKDLIAINA

SEQ ID NO: 259 - protein, 10 aa, mycoplasma genitalium
  Sequence located in MG025 at 100-109 and may interact with Sequence 260 in this patent.
YFLKDSFKKV

SEQ ID NO: 260 - protein, 10 aa, mycoplasma genitalium
  Sequence located in MG095 at 385-394 and may interact with Sequence 259 in this patent.
DFFKAIFQKV

SEQ ID NO: 261 - protein, 10 aa, mycoplasma genitalium
  Sequence located in MG025 at 56-65 and may interact with Sequence 262 in this patent.
DYLIKYSNIS

SEQ ID NO: 262 - protein, 10 aa, mycoplasma genitalium
  Sequence located in MG180 at 267-276 and may interact with Sequence 261 in this patent.
VIKDLIAINA

SEQ ID NO: 263 - protein, 10 aa, mycoplasma genitalium
  Sequence located in MG027 at 94-103 and may interact with Sequence 264 in this patent.
SGKKIDLLTT

SEQ ID NO: 264 - protein, 10 aa, mycoplasma genitalium
  Sequence located in MG027 at 94-103 and may interact with Sequence 263 in this patent.
SGKKIDLLTT

SEQ ID NO: 265 - protein, 10 aa, mycoplasma genitalium
  Sequence located in MG028 at 157-166 and may interact with Sequence 266 in this patent.
EILKAKKGEP

SEQ ID NO: 266 - protein, 10 aa, mycoplasma genitalium
  Sequence located in MG335.2 at 276-285 and may interact with Sequence 265 in this patent.
WFALFSFEYF

SEQ ID NO: 267 - protein, 10 aa, mycoplasma genitalium
  Sequence located in MG029 at 101-110 and may interact with Sequence 268 in this patent.
ICDTPNVLFK

SEQ ID NO: 268 - protein, 10 aa, mycoplasma genitalium
  Sequence located in MG130 at 180-189 and may interact with Sequence 267 in this patent.
NGISGINKKL

SEQ ID NO: 269 - protein, 10 aa, mycoplasma genitalium
  Sequence located in MG030 at 195-204 and may interact with Sequence 270 in this patent.
LGDAGDRLFG

SEQ ID NO: 270 - protein, 10 aa, mycoplasma genitalium
  Sequence located in MG081 at 2-11 and may interact with Sequence 269 in this patent.
AKKTVTRIAK

SEQ ID NO: 271 - protein, 10 aa, mycoplasma genitalium
  Sequence located in MG030 at 141-150 and may interact with Sequence 272 in this patent.
IKSIKEDKPI

SEQ ID NO: 272 - protein, 10 aa, mycoplasma genitalium
  Sequence located in MG147 at 303-312 and may interact with Sequence 271 in this patent.
YFGYFLILGN

SEQ ID NO: 273 - protein, 10 aa, mycoplasma genitalium
  Sequence located in MG031 at 428-437 and may interact with Sequence 274 in this patent.
GRYDDVIEFS

SEQ ID NO: 274 - protein, 10 aa, mycoplasma genitalium
  Sequence located in MG206 at 196-205 and may interact with Sequence 273 in this patent.
PSVVINYLKA

SEQ ID NO: 275 - protein, 10 aa, mycoplasma genitalium
  Sequence located in MG031 at 31-40 and may interact with Sequence 276 in this patent.
LNSLIERIES

SEQ ID NO: 276 - protein, 10 aa, mycoplasma genitalium
  Sequence located in MG217 at 226-235 and may interact with Sequence 275 in this patent.
ALYSLDKTIQ

SEQ ID NO: 277 - protein, 10 aa, mycoplasma genitalium
  Sequence located in MG031 at 32-41 and may interact with Sequence 278 in this patent.
NSLIERIESI

SEQ ID NO: 278 - protein, 10 aa, mycoplasma genitalium
  Sequence located in MG217 at 225-234 and may interact with Sequence 277 in this patent.
NALYSLDKTI

SEQ ID NO: 279 - protein, 10 aa, mycoplasma genitalium
  Sequence located in MG035 at 143-152 and may interact with Sequence 280 in this patent.
DTLLFANSLL

SEQ ID NO: 280 - protein, 10 aa, mycoplasma genitalium
  Sequence located in MG107 at 43-52 and may interact with Sequence 279 in this patent.
KKRISEKEGI

SEQ ID NO: 281 - protein, 10 aa, mycoplasma genitalium
  Sequence located in MG036 at 109-118 and may interact with Sequence 282 in this patent.
EEVRLKYRYL

SEQ ID NO: 282 - protein, 10 aa, mycoplasma genitalium
  Sequence located in MG085 at 107-116 and may interact with Sequence 281 in this patent.
QVPILKTNLF

SEQ ID NO: 283 - protein, 10 aa, mycoplasma genitalium
  Sequence located in MG036 at 207-216 and may interact with Sequence 284 in this patent.
YRDEDSRKDR

SEQ ID NO: 284 - protein, 10 aa, mycoplasma genitalium
  Sequence located in MG443 at 252-261 and may interact with Sequence 283 in this patent.
IAILIGSFVA

SEQ ID NO: 285 - protein, 10 aa, mycoplasma genitalium
  Sequence located in MG038 at 416-425 and may interact with Sequence 286 in this patent.
ADGGIVKSNY

SEQ ID NO: 286 - protein, 10 aa, mycoplasma genitalium
  Sequence located in MG462 at 191-200 and may interact with Sequence 285 in this patent.
GVATYNFAVV

SEQ ID NO: 287 - protein, 10 aa, mycoplasma genitalium
  Sequence located in MG042 at 237-246 and may interact with Sequence 288 in this patent.
QYQKKKTELK

SEQ ID NO: 288 - protein, 10 aa, mycoplasma genitalium
  Sequence located in MG319 at 8-17 and may interact with Sequence 287 in this patent.
FKLCFLLLVL

SEQ ID NO: 289 - protein, 10 aa, mycoplasma genitalium
  Sequence located in MG042 at 81-90 and may interact with Sequence 290 in this patent.
FATVFQDYAL

SEQ ID NO: 290 - protein, 10 aa, mycoplasma genitalium
  Sequence located in MG379 at 132-141 and may interact with Sequence 289 in this patent.
KGVILKDGSE

SEQ ID NO: 291 - protein, 10 aa, mycoplasma genitalium
  Sequence located in MG042 at 49-58 and may interact with Sequence 292 in this patent.
LLRLLAGFED

SEQ ID NO: 292 - protein, 10 aa, mycoplasma genitalium
  Sequence located in MG455 at 317-326 and may interact with Sequence 291 in this patent.
KEAQQRSELI

SEQ ID NO: 293 - protein, 10 aa, mycoplasma genitalium
  Sequence located in MG043 at 124-133 and may interact with Sequence 294 in this patent.
LSLNRVGDNN

SEQ ID NO: 294 - protein, 10 aa, mycoplasma genitalium
  Sequence located in MG195 at 140-149 and may interact with Sequence 293 in this patent.
KTEVADAIII

SEQ ID NO: 295 - protein, 10 aa, mycoplasma genitalium
  Sequence located in MG044 at 174-183 and may interact with Sequence 296 in this patent.
LILASQDLGY

SEQ ID NO: 296 - protein, 10 aa, mycoplasma genitalium
  Sequence located in MG110 at 55-64 and may interact with Sequence 295 in this patent.
ITKVLARKNE

SEQ ID NO: 297 - protein, 10 aa, mycoplasma genitalium
  Sequence located in MG044 at 175-184 and may interact with Sequence 298 in this patent.
ILASQDLGYS

SEQ ID NO: 298 - protein, 10 aa, mycoplasma genitalium
  Sequence located in MG110 at 54-63 and may interact with Sequence 297 in this patent.
AITKVLARKN

SEQ ID NO: 299 - protein, 10 aa, mycoplasma genitalium
  Sequence located in MG044 at 176-185 and may interact with Sequence 300 in this patent.
LASQDLGYSP

SEQ ID NO: 300 - protein, 10 aa, mycoplasma genitalium
  Sequence located in MG110 at 53-62 and may interact with Sequence 299 in this patent.
GAITKVLARK

SEQ ID NO: 301 - protein, 10 aa, mycoplasma genitalium
  Sequence located in MG044 at 61-70 and may interact with Sequence 302 in this patent.
LRLGETDFAT

SEQ ID NO: 302 - protein, 10 aa, mycoplasma genitalium
  Sequence located in MG360 at 169-178 and may interact with Sequence 301 in this patent.
QAKPFGIKSC

SEQ ID NO: 303 - protein, 10 aa, mycoplasma genitalium
  Sequence located in MG044 at 154-163 and may interact with Sequence 304 in this patent.
FSVPYALILI

SEQ ID NO: 304 - protein, 10 aa, mycoplasma genitalium
  Sequence located in MG423 at 313-322 and may interact with Sequence 303 in this patent.
NEDERIRYRK

SEQ ID NO: 305 - protein, 10 aa, mycoplasma genitalium
  Sequence located in MG045 at 151-160 and may interact with Sequence 306 in this patent.
LVFVYRGEKI

SEQ ID NO: 306 - protein, 10 aa, mycoplasma genitalium
  Sequence located in MG135 at 214-223 and may interact with Sequence 305 in this patent.
EYKNVATFLN

SEQ ID NO: 307 - protein, 10 aa, mycoplasma genitalium
  Sequence located in MG045 at 285-294 and may interact with Sequence 308 in this patent.
LSEEQIPDGN

SEQ ID NO: 308 - protein, 10 aa, mycoplasma genitalium
  Sequence located in MG152 at 185-194 and may interact with Sequence 307 in this patent.
VSVRDLLLAK

SEQ ID NO: 309 - protein, 10 aa, mycoplasma genitalium
  Sequence located in MG045 at 438-447 and may interact with Sequence 310 in this patent.
VETIKKAYTI

SEQ ID NO: 310 - protein, 10 aa, mycoplasma genitalium
  Sequence located in MG255 at 254-263 and may interact with Sequence 309 in this patent.
NGVCFFYGFD

SEQ ID NO: 311 - protein, 10 aa, mycoplasma genitalium
  Sequence located in MG046 at 133-142 and may interact with Sequence 312 in this patent.
GLVISGGHTA

SEQ ID NO: 312 - protein, 10 aa, mycoplasma genitalium
  Sequence located in MG192 at 248-257 and may interact with Sequence 311 in this patent.
AKNDAPSVSG

SEQ ID NO: 313 - protein, 10 aa, mycoplasma genitalium
  Sequence located in MG046 at 132-141 and may interact with Sequence 314 in this patent.
LGLVISGGHT

SEQ ID NO: 314 - protein, 10 aa, mycoplasma genitalium
  Sequence located in MG192 at 247-256 and may interact with Sequence 313 in this patent.
EAKNDAPSVS

SEQ ID NO: 315 - protein, 10 aa, mycoplasma genitalium
  Sequence located in MG064 at 1096-1105 and may interact with Sequence 316 in this patent.
SGIFPATSSF

SEQ ID NO: 316 - protein, 10 aa, mycoplasma genitalium
  Sequence located in MG176 at 80-89 and may interact with Sequence 315 in this patent.
KGTGRGKDTT

SEQ ID NO: 317 - protein, 10 aa, mycoplasma genitalium
  Sequence located in MG064 at 716-725 and may interact with Sequence 318 in this patent.
GTTDKRSESS

SEQ ID NO: 318 - protein, 10 aa, mycoplasma genitalium
  Sequence located in MG192 at 926-935 and may interact with Sequence 317 in this patent.
PSRVFAGFAA

SEQ ID NO: 319 - protein, 10 aa, mycoplasma genitalium
  Sequence located in MG064 at 605-614 and may interact with Sequence 320 in this patent.
LKKDVVKILK

SEQ ID NO: 320 - protein, 10 aa, mycoplasma genitalium
  Sequence located in MG199 at 60-69 and may interact with Sequence 319 in this patent.
ELFVNYLNKL

SEQ ID NO: 321 - protein, 10 aa, mycoplasma genitalium
  Sequence located in MG064 at 1288-1297 and may interact with Sequence 322 in this patent.
LDAFLSFESA

SEQ ID NO: 322 - protein, 10 aa, mycoplasma genitalium
  Sequence located in MG231 at 611-620 and may interact with Sequence 321 in this patent.
EVRKEGKLGR

SEQ ID NO: 323 - protein, 10 aa, mycoplasma genitalium
  Sequence located in MG076 at 125-134 and may interact with Sequence 324 in this patent.
YLVGSLVSGL

SEQ ID NO: 324 - protein, 10 aa, mycoplasma genitalium
  Sequence located in MG346 at 85-94 and may interact with Sequence 323 in this patent.
KSGDKTPDQI

SEQ ID NO: 325 - protein, 10 aa, mycoplasma genitalium
  Sequence located in MG085 at 242-251 and may interact with Sequence 326 in this patent.
SFERLGSDLK

SEQ ID NO: 326 - protein, 10 aa, mycoplasma genitalium
  Sequence located in MG457 at 221-230 and may interact with Sequence 325 in this patent.
AKLAKSTVKF

SEQ ID NO: 327 - protein, 10 aa, mycoplasma genitalium
  Sequence located in MG086 at 117-126 and may interact with Sequence 328 in this patent.
NKTKTIAELL

SEQ ID NO: 328 - protein, 10 aa, mycoplasma genitalium
  Sequence located in MG214 at 76-85 and may interact with Sequence 327 in this patent.
KKFGNSFSLV

SEQ ID NO: 329 - protein, 10 aa, mycoplasma genitalium
  Sequence located in MG086 at 46-55 and may interact with Sequence 330 in this patent.
LQDNSFYFFI

SEQ ID NO: 330 - protein, 10 aa, mycoplasma genitalium
  Sequence located in MG379 at 128-137 and may interact with Sequence 329 in this patent.
NEKIKGVILK

SEQ ID NO: 331 - protein, 10 aa, mycoplasma genitalium
  Sequence located in MG088 at 3-12 and may interact with Sequence 332 in this patent.
KNRALKRTVL

SEQ ID NO: 332 - protein, 10 aa, mycoplasma genitalium
  Sequence located in MG088 at 3-12 and may interact with Sequence 331 in this patent.
KNRALKRTVL

SEQ ID NO: 333 - protein, 10 aa, mycoplasma genitalium
  Sequence located in MG088 at 4-13 and may interact with Sequence 334 in this patent.
NRALKRTVLP

SEQ ID NO: 334 - protein, 10 aa, mycoplasma genitalium
  Sequence located in MG088 at 2-11 and may interact with Sequence 333 in this patent.
RKNRALKRTV

SEQ ID NO: 335 - protein, 10 aa, mycoplasma genitalium
  Sequence located in MG090 at 112-121 and may interact with Sequence 336 in this patent.
LTKYNEIIAS

SEQ ID NO: 336 - protein, 10 aa, mycoplasma genitalium
  Sequence located in MG361 at 23-32 and may interact with Sequence 335 in this patent.
KGFVIFDYSG

SEQ ID NO: 337 - protein, 10 aa, mycoplasma genitalium
  Sequence located in MG093 at 133-142 and may interact with Sequence 338 in this patent.
EITATLTVIV

SEQ ID NO: 338 - protein, 10 aa, mycoplasma genitalium
  Sequence located in MG200 at 448-457 and may interact with Sequence 337 in this patent.
NDHGKGCGDL

SEQ ID NO: 339 - protein, 10 aa, mycoplasma genitalium
  Sequence located in MG093 at 118-127 and may interact with Sequence 340 in this patent.
RLQFGEHKLI

SEQ ID NO: 340 - protein, 10 aa, mycoplasma genitalium
  Sequence located in MG370 at 287-296 and may interact with Sequence 339 in this patent.
YELVFPKLET

SEQ ID NO: 341 - protein, 10 aa, mycoplasma genitalium
  Sequence located in MG094 at 359-368 and may interact with Sequence 342 in this patent.
EERKDSRPIL

SEQ ID NO: 342 - protein, 10 aa, mycoplasma genitalium
  Sequence located in MG140 at 768-777 and may interact with Sequence 341 in this patent.
KNRSRILTLL

SEQ ID NO: 343 - protein, 10 aa, mycoplasma genitalium
  Sequence located in MG095 at 181-190 and may interact with Sequence 344 in this patent.
TATSSTRAST

SEQ ID NO: 344 - protein, 10 aa, mycoplasma genitalium
  Sequence located in MG391 at 390-399 and may interact with Sequence 343 in this patent.
STSARGAGSS

SEQ ID NO: 345 - protein, 10 aa, mycoplasma genitalium
  Sequence located in MG096 at 362-371 and may interact with Sequence 346 in this patent.
AEEKRKAEEA

SEQ ID NO: 346 - protein, 10 aa, mycoplasma genitalium
  Sequence located in MG411 at 467-476 and may interact with Sequence 345 in this patent.
GLFGLSFFLR

SEQ ID NO: 347 - protein, 10 aa, mycoplasma genitalium
  Sequence located in MG098 at 147-156 and may interact with Sequence 348 in this patent.
IAGILREVLI

SEQ ID NO: 348 - protein, 10 aa, mycoplasma genitalium
  Sequence located in MG372 at 115-124 and may interact with Sequence 347 in this patent.
DKNFAENSSN

SEQ ID NO: 349 - protein, 10 aa, mycoplasma genitalium
  Sequence located in MG098 at 148-157 and may interact with Sequence 350 in this patent.
AGILREVLIS

SEQ ID NO: 350 - protein, 10 aa, mycoplasma genitalium
  Sequence located in MG372 at 114-123 and may interact with Sequence 349 in this patent.
RDKNFAENSS

SEQ ID NO: 351 - protein, 10 aa, mycoplasma genitalium
  Sequence located in MG098 at 149-158 and may interact with Sequence 352 in this patent.
GILREVLIST

SEQ ID NO: 352 - protein, 10 aa, mycoplasma genitalium
  Sequence located in MG372 at 113-122 and may interact with Sequence 351 in this patent.
RRDKNFAENS

SEQ ID NO: 353 - protein, 10 aa, mycoplasma genitalium
  Sequence located in MG099 at 139-148 and may interact with Sequence 354 in this patent.
IAGGSSSGSA

SEQ ID NO: 354 - protein, 10 aa, mycoplasma genitalium
  Sequence located in MG391 at 389-398 and may interact with Sequence 353 in this patent.
NSTSARGAGS

SEQ ID NO: 355 - protein, 10 aa, mycoplasma genitalium
  Sequence located in MG099 at 138-147 and may interact with Sequence 356 in this patent.
LIAGGSSSGS

SEQ ID NO: 356 - protein, 10 aa, mycoplasma genitalium
  Sequence located in MG391 at 388-397 and may interact with Sequence 355 in this patent.
QNSTSARGAG

SEQ ID NO: 357 - protein, 10 aa, mycoplasma genitalium
  Sequence located in MG101 at 73-82 and may interact with Sequence 358 in this patent.
FVCKDVGFSF

SEQ ID NO: 358 - protein, 10 aa, mycoplasma genitalium
  Sequence located in MG140 at 8-17 and may interact with Sequence 357 in this patent.
KNRLVNSKTK

SEQ ID NO: 359 - protein, 10 aa, mycoplasma genitalium
  Sequence located in MG103 at 136-145 and may interact with Sequence 360 in this patent.
LLKLFSFFKK

SEQ ID NO: 360 - protein, 10 aa, mycoplasma genitalium
  Sequence located in MG307 at 778-787 and may interact with Sequence 359 in this patent.
QQFEKAKKLF

SEQ ID NO: 361 - protein, 10 aa, mycoplasma genitalium
  Sequence located in MG104 at 402-411 and may interact with Sequence 362 in this patent.
CDETVINSLK

SEQ ID NO: 362 - protein, 10 aa, mycoplasma genitalium
  Sequence located in MG212 at 172-181 and may interact with Sequence 361 in this patent.
RILSNDIGEF

SEQ ID NO: 363 - protein, 10 aa, mycoplasma genitalium
  Sequence located in MG104 at 619-628 and may interact with Sequence 364 in this patent.
TEIKIINCER

SEQ ID NO: 364 - protein, 10 aa, mycoplasma genitalium
  Sequence located in MG345 at 327-336 and may interact with Sequence 363 in this patent.
AFGIDDFYLC

SEQ ID NO: 365 - protein, 10 aa, mycoplasma genitalium
  Sequence located in MG111 at 154-163 and may interact with Sequence 366 in this patent.
FRNLLNKRYG

SEQ ID NO: 366 - protein, 10 aa, mycoplasma genitalium
  Sequence located in MG204 at 11-20 and may interact with Sequence 365 in this patent.
KAIEEVFAVS

SEQ ID NO: 367 - protein, 10 aa, mycoplasma genitalium
  Sequence located in MG113 at 276-285 and may interact with Sequence 368 in this patent.
SNDIISNLKT

SEQ ID NO: 368 - protein, 10 aa, mycoplasma genitalium
  Sequence located in MG229 at 266-275 and may interact with Sequence 367 in this patent.
GIVDDAIKFS

SEQ ID NO: 369 - protein, 10 aa, mycoplasma genitalium
  Sequence located in MG113 at 423-432 and may interact with Sequence 370 in this patent.
GFGLGFDRLL

SEQ ID NO: 370 - protein, 10 aa, mycoplasma genitalium
  Sequence located in MG386 at 68-77 and may interact with Sequence 369 in this patent.
KETVKSKSKS

SEQ ID NO: 371 - protein, 10 aa, mycoplasma genitalium
  Sequence located in MG114 at 51-60 and may interact with Sequence 372 in this patent.
LDNQLGVLAN

SEQ ID NO: 372 - protein, 10 aa, mycoplasma genitalium
  Sequence located in MG227 at 253-262 and may interact with Sequence 371 in this patent.
QVVLKSDKSI

SEQ ID NO: 373 - protein, 10 aa, mycoplasma genitalium
  Sequence located in MG115 at 99-108 and may interact with Sequence 374 in this patent.
AGSKAVENKA

SEQ ID NO: 374 - protein, 10 aa, mycoplasma genitalium
  Sequence located in MG348 at 57-66 and may interact with Sequence 373 in this patent.
STTFRNLVFG

SEQ ID NO: 375 - protein, 10 aa, mycoplasma genitalium
  Sequence located in MG115 at 98-107 and may interact with Sequence 376 in this patent.
IAGSKAVENK

SEQ ID NO: 376 - protein, 10 aa, mycoplasma genitalium
  Sequence located in MG348 at 56-65 and may interact with Sequence 375 in this patent.
NSTTFRNLVF

SEQ ID NO: 377 - protein, 10 aa, mycoplasma genitalium
  Sequence located in MG117 at 46-55 and may interact with Sequence 378 in this patent.
FLIFNNFNKL

SEQ ID NO: 378 - protein, 10 aa, mycoplasma genitalium
  Sequence located in MG252 at 84-93 and may interact with Sequence 377 in this patent.
QLVKVVENKK

SEQ ID NO: 379 - protein, 10 aa, mycoplasma genitalium
  Sequence located in MG118 at 262-271 and may interact with Sequence 380 in this patent.
NLGSGIGTSN

SEQ ID NO: 380 - protein, 10 aa, mycoplasma genitalium
  Sequence located in MG431 at 67-76 and may interact with Sequence 379 in this patent.
IESGSYTGTV

SEQ ID NO: 381 - protein, 10 aa, mycoplasma genitalium
  Sequence located in MG119 at 516-525 and may interact with Sequence 382 in this patent.
NKAILLVSYE

SEQ ID NO: 382 - protein, 10 aa, mycoplasma genitalium
  Sequence located in MG370 at 261-270 and may interact with Sequence 381 in this patent.
LVGDKKYSLI

SEQ ID NO: 383 - protein, 10 aa, mycoplasma genitalium
  Sequence located in MG119 at 517-526 and may interact with Sequence 384 in this patent.
KAILLVSYEL

SEQ ID NO: 384 - protein, 10 aa, mycoplasma genitalium
  Sequence located in MG370 at 260-269 and may interact with Sequence 383 in this patent.
QLVGDKKYSL

SEQ ID NO: 385 - protein, 10 aa, mycoplasma genitalium
  Sequence located in MG120 at 305-314 and may interact with Sequence 386 in this patent.
SAISAVPATG

SEQ ID NO: 386 - protein, 10 aa, mycoplasma genitalium
  Sequence located in MG384 at 34-43 and may interact with Sequence 385 in this patent.
PGGGNGGNGG

SEQ ID NO: 387 - protein, 10 aa, mycoplasma genitalium
  Sequence located in MG121 at 123-132 and may interact with Sequence 388 in this patent.
FSDTTLRVRY

SEQ ID NO: 388 - protein, 10 aa, mycoplasma genitalium
  Sequence located in MG355 at 525-534 and may interact with Sequence 387 in this patent.
EAVRRKPYSV

SEQ ID NO: 389 - protein, 10 aa, mycoplasma genitalium
  Sequence located in MG122 at 480-489 and may interact with Sequence 390 in this patent.
KFSFEKISVN

SEQ ID NO: 390 - protein, 10 aa, mycoplasma genitalium
  Sequence located in MG434 at 85-94 and may interact with Sequence 389 in this patent.
VNGYFLKAKL

SEQ ID NO: 391 - protein, 10 aa, mycoplasma genitalium
  Sequence located in MG122 at 639-648 and may interact with Sequence 392 in this patent.
GCSDFPKCKY

SEQ ID NO: 392 - protein, 10 aa, mycoplasma genitalium
  Sequence located in MG440 at 99-108 and may interact with Sequence 391 in this patent.
STGVKGLRLV

SEQ ID NO: 393 - protein, 10 aa, mycoplasma genitalium
  Sequence located in MG127 at 79-88 and may interact with Sequence 394 in this patent.
TNSVSVEELS

SEQ ID NO: 394 - protein, 10 aa, mycoplasma genitalium
  Sequence located in MG209 at 259-268 and may interact with Sequence 393 in this patent.
GQFLHANRIC

SEQ ID NO: 395 - protein, 10 aa, mycoplasma genitalium
  Sequence located in MG127 at 80-89 and may interact with Sequence 396 in ihis patent.
NSVSVEELSI

SEQ ID NO: 396 - protein, 10 aa, mycoplasma genitalium
  Sequence located in MG209 at 258-267 and may interact with Sequence 395 in this patent.
YGQFLHANRI

SEQ ID NO: 397 - protein, 10 aa, mycoplasma genitalium
  Sequence located in MG127 at 57-66 and may interact with Sequence 398 in this patent.
ILSLSEDGTE

SEQ ID NO: 398 - protein, 10 aa, mycoplasma genitalium
  Sequence located in MG430 at 350-359 and may interact with Sequence 397 in this patent.
NETKTLIPSL

SEQ ID NO: 399 - protein, 10 aa, mycoplasma genitalium
  Sequence located in MG128 at 151-160 and may interact with Sequence 400 in this patent.
LAKSAKGAVI

SEQ ID NO: 400 - protein, 10 aa, mycoplasma genitalium
  Sequence located in MG463 at 146-155 and may interact with Sequence 399 in this patent.
DYSAFGAFCQ

SEQ ID NO: 401 - protein, 10 aa, mycoplasma genitalium
  Sequence located in MG130 at 385-394 and may interact with Sequence 402 in this patent.
ARIGARSDSS

SEQ ID NO: 402 - protein, 10 aa, mycoplasma genitalium
  Sequence located in MG130 at 385-394 and may interact with Sequence 401 in this patent.
ARIGARSDSS

SEQ ID NO: 403 - protein, 10 aa, mycoplasma genitalium
  Sequence located in MG130 at 207-216 and may interact with Sequence 404 in this patent.
KYEVNVLTES

SEQ ID NO: 404 - protein, 10 aa, mycoplasma genitalium
  Sequence located in MG360 at 390-399 and may interact with Sequence 403 in this patent.
LIFDINESFG

SEQ ID NO: 405 - protein, 10 aa, mycoplasma genitalium
  Sequence located in MG132 at 4-13 and may interact with Sequence 406 in this patent.
NTTSSCIFCD

SEQ ID NO: 406 - protein, 10 aa, mycoplasma genitalium
  Sequence located in MG182 at 46-55 and may interact with Sequence 405 in this patent.
IGSGRTDKGV

SEQ ID NO: 407 - protein, 10 aa, mycoplasma genitalium
  Sequence located in MG134 at 70-79 and may interact with Sequence 408 in this patent.
IIEATNEAVS

SEQ ID NO: 408 - protein, 10 aa, mycoplasma genitalium
  Sequence located in MG189 at 100-109 and may interact with Sequence 407 in this patent.
DNFSRVLSDG

SEQ ID NO: 409 - protein, 10 aa, mycoplasma genitalium
  Sequence located in MG135 at 132-141 and may interact with Sequence 410 in this patent.
DIVNFIFVGT

SEQ ID NO: 410 - protein, 10 aa, mycoplasma genitalium
  Sequence located in MG468 at 1500-1509 and may interact with Sequence 409 in this patent.
SSDEYEIDYV

SEQ ID NO: 411 - protein, 10 aa, mycoplasma genitalium
  Sequence located in MG136 at 286-295 and may interact with Sequence 412 in this patent.
SLCESLNQFS

SEQ ID NO: 412 - protein, 10 aa, mycoplasma genitalium
  Sequence located in MG157 at 138-147 and may interact with Sequence 411 in this patent.
RQALTKVLKA

SEQ ID NO: 413 - protein, 10 aa, mycoplasma genitalium
  Sequence located in MG139 at 202-211 and may interact with Sequence 414 in this patent.
SESTNAEVPG

SEQ ID NO: 414 - protein, 10 aa, mycoplasma genitalium
  Sequence located in MG411 at 517-526 and may interact with Sequence 413 in this patent.
SWDLRISAFA

SEQ ID NO: 415 - protein, 10 aa, mycoplasma genitalium
  Sequence located in MG139 at 293-302 and may interact with Sequence 416 in this patent.
IKNYPDRNIL

SEQ ID NO: 416 - protein, 10 aa, mycoplasma genitalium
  Sequence located in MG450 at 32-41 and may interact with Sequence 415 in this patent.
DLIIGIPIDK

SEQ ID NO: 417 - protein, 10 aa, mycoplasma genitalium
  Sequence located in MG140 at 264-273 and may interact with Sequence 418 in this patent.
LLKDRRDLLL

SEQ ID NO: 418 - protein, 10 aa, mycoplasma genitalium
  Sequence located in MG218 at 651-660 and may interact with Sequence 417 in this patent.
KELVASVEKQ

SEQ ID NO: 419 - protein, 10 aa, mycoplasma genitalium
  Sequence located in MG140 at 329-338 and may interact with Sequence 420 in this patent.
NLVLNNKKVL

SEQ ID NO: 420 - protein, 10 aa, mycoplasma genitalium
  Sequence located in MG390 at 598-607 and may interact with Sequence 419 in this patent.
IKNKVILLDE

SEQ ID NO: 421 - protein, 10 aa, mycoplasma genitalium
  Sequence located in MG140 at 328-337 and may interact with Sequence 422 in this patent.
VNLVLNNKKV

SEQ ID NO: 422 - protein, 10 aa, mycoplasma genitalium
  Sequence located in MG390 at 597-606 and may interact with Sequence 421 in this patent.
NIKNKVILLD

SEQ ID NO: 423 - protein, 10 aa, mycoplasma genitalium
  Sequence located in MG141 at 508-517 and may interact with Sequence 424 in this patent.
KKDQVDNNQT

SEQ ID NO: 424 - protein, 10 aa, mycoplasma genitalium
  Sequence located in MG302 at 34-43 and may interact with Sequence 423 in this patent.
GLVIINLVFL

SEQ ID NO: 425 - protein, 10 aa, mycoplasma genitalium
  Sequence located in MG141 at 164-173 and may interact with Sequence 426 in this patent.
VGEVIEAKVE

SEQ ID NO: 426 - protein, 10 aa, mycoplasma genitalium
  Sequence located in MG316 at 206-215 and may interact with Sequence 425 in this patent.
HALNNFGFNF

SEQ ID NO: 427 - protein, 10 aa, mycoplasma genitalium
  Sequence located in MG144 at 37-46 and may interact with Sequence 428 in this patent.
DFEKTVTLTR

SEQ ID NO: 428 - protein, 10 aa, mycoplasma genitalium
  Sequence located in MG274 at 252-261 and may interact with Sequence 427 in this patent.
IEFFSYRQGP

SEQ ID NO: 429 - protein, 10 aa, mycoplasma genitalium
  Sequence located in MG146 at 65-74 and may interact with Sequence 430 in this patent.
NHYSSCLITI

SEQ ID NO: 430 - protein, 10 aa, mycoplasma genitalium
  Sequence located in MG468.1 at 235-244 and may interact with Sequence 429 in this patent.
NRDKGTTIVI

SEQ ID NO: 431 - protein, 10 aa, mycoplasma genitalium
  Sequence located in MG147 at 64-73 and may interact with Sequence 432 in this patent.
EEIIVFVVGS

SEQ ID NO: 432 - protein, 10 aa, mycoplasma genitalium
  Sequence located in MG266 at 340-349 and may interact with Sequence 431 in this patent.
GAHNENDNFF

SEQ ID NO: 433 - protein, 10 aa, mycoplasma genitalium
  Sequence located in MG148 at 213-222 and may interact with Sequence 434 in this patent.
ADYIFSFFGE

SEQ ID NO: 434 - protein, 10 aa, mycoplasma genitalium
  Sequence located in MG233 at 19-28 and may interact with Sequence 433 in this patent.
FAEKGKDIVC

SEQ ID NO: 435 - protein, 10 aa, mycoplasma genitalium
  Sequence located in MG148 at 214-223 and may interact with Sequence 436 in this patent.
DYIFSFFGEK

SEQ ID NO: 436 - protein, 10 aa, mycoplasma genitalium
  Sequence located in MG233 at 18-27 and may interact with Sequence 435 in this patent.
LFAEKGKDIV

SEQ ID NO: 437 - protein, 10 aa, mycoplasma genitalium
  Sequence located in MG149 at 180-189 and may interact with Sequence 438 in this patent.
IFLVIDYKKD

SEQ ID NO: 438 - protein, 10 aa, mycoplasma genitalium
  Sequence located in MG149 at 180-189 and may interact with Sequence 437 in this patent.
IFLVIDYKKD

SEQ ID NO: 439 - protein, 10 aa, mycoplasma genitalium
  Sequence located in MG149 at 181-190 and may interact with Sequence 440 in this patent.
FLVIDYKKDS

SEQ ID NO: 440 - protein, 10 aa, mycoplasma genitalium
  Sequence located in MG149 at 179-188 and may interact with Sequence 439 in this patent.
GIFLVIDYKK

SEQ ID NO: 441 - protein, 10 aa, mycoplasma genitalium
  Sequence located in MG158 at 16-25 and may interact with Sequence 442 in this patent.
SYEGHTKGNG

SEQ ID NO: 442 - protein, 10 aa, mycoplasma genitalium
  Sequence located in MG407 at 118-127 and may interact with Sequence 441 in this patent.
AILSVSLAVS

SEQ ID NO: 443 - protein, 10 aa, mycoplasma genitalium
  Sequence located in MG158 at 15-24 and may interact with Sequence 444 in this patent.
VSYEGHTKGN

SEQ ID NO: 444 - protein, 10 aa, mycoplasma genitalium
  Sequence located in MG407 at 117-126 and may interact with Sequence 443 in this patent.
NAILSVSLAV

SEQ ID NO: 445 - protein, 10 aa, mycoplasma genitalium
  Sequence located in MG161 at 14-23 and may interact with Sequence 446 in this patent.
TGAKQVGIIK

SEQ ID NO: 446 - protein, 10 aa, mycoplasma genitalium
  Sequence located in MG422 at 23-32 and may interact with Sequence 445 in this patent.
RTSFLDANYF

SEQ ID NO: 447 - protein, 10 aa, mycoplasma genitalium
  Sequence located in MG161 at 13-22 and may interact with Sequence 448 in this patent.
NTGAKQVGII

SEQ ID NO: 448 - protein, 10 aa, mycoplasma genitalium
  Sequence located in MG422 at 22-31 and may interact with Sequence 447 in this patent.
IRTSFLDANY

SEQ ID NO: 449 - protein, 10 aa, mycoplasma genitalium
  Sequence located in MG162 at 20-29 and may interact with Sequence 450 in this patent.
GSGIVLKVLT

SEQ ID NO: 450 - protein, 10 aa, mycoplasma genitalium
  Sequence located in MG334 at 430-439 and may interact with Sequence 449 in this patent.
SKNLQNYTRS

SEQ ID NO: 451 - protein, 10 aa, mycoplasma genitalium
  Sequence located in MG163 at 98-107 and may interact with Sequence 452 in this patent.
SFLEKLIYIA

SEQ ID NO: 452 - protein, 10 aa, mycoplasma genitalium
  Sequence located in MG307 at 835-844 and may interact with Sequence 451 in this patent.
GNIDELFKKA

SEQ ID NO: 453 - protein, 10 aa, mycoplasma genitalium
  Sequence located in MG167 at 95-104 and may interact with Sequence 454 in this patent.
SKYHGRIAAL

SEQ ID NO: 454 - protein, 10 aa, mycoplasma genitalium
  Sequence located in MG251 at 326-335 and may interact with Sequence 453 in this patent.
ALIVSSYRRE

SEQ ID NO: 455 - protein, 10 aa, mycoplasma genitalium
  Sequence located in MG169 at 40-49 and may interact with Sequence 456 in this patent.
KARKSGLTRL

SEQ ID NO: 456 - protein, 10 aa, mycoplasma genitalium
  Sequence located in MG169 at 40-49 and may interact with Sequence 455 in this patent.
KARKSGLTRL

SEQ ID NO: 457 - protein, 10 aa, mycoplasma genitalium
  Sequence located in MG170 at 73-82 and may interact with Sequence 458 in this patent.
GGGGLNQLSL

SEQ ID NO: 458 - protein, 10 aa, mycoplasma genitalium
  Sequence located in MG180 at 235-244 and may interact with Sequence 457 in this patent.
KGKLVKAASP

SEQ ID NO: 459 - protein, 10 aa, mycoplasma genitalium
  Sequence located in MG171 at 52-61 and may interact with Sequence 460 in this patent.
RKIAAIISQG

SEQ ID NO: 460 - protein, 10 aa, mycoplasma genitalium
  Sequence located in MG468 at 75-84 and may interact with Sequence 459 in this patent.
SLTNDSGYFS

SEQ ID NO: 461 - protein, 10 aa, mycoplasma genitalium
  Sequence located in MG171 at 53-62 and may interact with Sequence 462 in this patent.
KIAAIISQGG

SEQ ID NO: 462 - protein, 10 aa, mycoplasma genitalium
  Sequence located in MG468 at 74-83 and may interact with Sequence 461 in this patent.
ASLTNDSGYF

SEQ ID NO: 463 - protein, 10 aa, mycoplasma genitalium
  Sequence located in MG171 at 54-63 and may interact with Sequence 464 in this patent.
IAAIISQGGY

SEQ ID NO: 464 - protein, 10 aa, mycoplasma genitalium
  Sequence located in MG468 at 73-82 and may interact with Sequence 463 in this patent.
IASLTNDSGY

SEQ ID NO: 465 - protein, 10 aa, mycoplasma genitalium
  Sequence located in MG171 at 55-64 and may interact with Sequence 466 in this patent.
AAIISQGGYV

SEQ ID NO: 466 - protein, 10 aa, mycoplasma genitalium
  Sequence located in MG468 at 72-81 and may interact with Sequence 465 in this patent.
YIASLTNDSG

SEQ ID NO: 467 - protein, 10 aa, mycoplasma genitalium
  Sequence located in MG171 at 56-65 and may interact with Sequence 468 in this patent.
AIISQGGYVD

SEQ ID NO: 468 - protein, 10 aa, mycoplasma genitalium
  Sequence located in MG468 at 71-80 and may interact with Sequence 467 in this patent.
VYIASLTNDS

SEQ ID NO: 469 - protein, 10 aa, mycoplasma genitalium
  Sequence located in MG172 at 27-36 and may interact with Sequence 470 in this patent.
FTIEKLLGKK

SEQ ID NO: 470 - protein, 10 aa, mycoplasma genitalium
  Sequence located in MG328 at 45-54 and may interact with Sequence 469 in this patent.
ERNLLEKTFF

SEQ ID NO: 471 - protein, 10 aa, mycoplasma genitalium
  Sequence located in MG149.1 at 51-60 and may interact with Sequence 472 in this patent.
ALLIYLLASV

SEQ ID NO: 472 - protein, 10 aa, mycoplasma genitalium
  Sequence located in MG341 at 564-573 and may interact with Sequence 471 in this patent.
NRGQQINEEG

SEQ ID NO: 473 - protein, 10 aa, mycoplasma genitalium
  Sequence located in MG149.1 at 52-61 and may interact with Sequence 474 in this patent.
LLIYLLASVD

SEQ ID NO: 474 - protein, 10 aa, mycoplasma genitalium
  Sequence located in MG341 at 563-572 and may interact with Sequence 473 in this patent.
VNRGQQINEE

SEQ ID NO: 475 - protein, 10 aa, mycoplasma genitalium
  Sequence located in MG174 at 27-36 and may interact with Sequence 476 in this patent.
CKTKKHKQRQ

SEQ ID NO: 476 - protein, 10 aa, mycoplasma genitalium
  Sequence located in MG323.1 at 137-146 and may interact with Sequence 475 in this patent.
LALLVFLSFR

SEQ ID NO: 477 - protein, 10 aa, mycoplasma genitalium
  Sequence located in MG174 at 28-37 and may interact with Sequence 478 in this patent.
KTKKHKQRQG

SEQ ID NO: 478 - protein, 10 aa, mycoplasma genitalium
  Sequence located in MG323.1 at 136-145 and may interact with Sequence 477 in this patent.
SLALLVFLSF

SEQ ID NO: 479 - protein, 10 aa, mycoplasma genitalium
  Sequence located in MG176 at 58-67 and may interact with Sequence 480 in this patent.
VAADKVAKTV

SEQ ID NO: 480 - protein, 10 aa, mycoplasma genitalium
  Sequence located in MG468 at 1392-1401 and may interact with Sequence 479 in this patent.
NSLRNLISSD

SEQ ID NO: 481 - protein, 10 aa, mycoplasma genitalium
  Sequence located in MG177 at 242-251 and may interact with Sequence 482 in this patent.
SVNESIKNLT

SEQ ID NO: 482 - protein, 10 aa, mycoplasma genitalium
  Sequence located in MG340 at 187-196 and may interact with Sequence 481 in this patent.
GYIFRNLIKS

SEQ ID NO: 483 - protein, 10 aa, mycoplasma genitalium
  Sequence located in MG179 at 62-71 and may interact with Sequence 484 in this patent.
AGEIKIFGKT

SEQ ID NO: 484 - protein, 10 aa, mycoplasma genitalium
  Sequence located in MG339 at 44-53 and may interact with Sequence 483 in this patent.
GSLNLDEALG

SEQ ID NO: 485 - protein, 10 aa, mycoplasma genitalium
  Sequence located in MG179 at 216-225 and may interact with Sequence 486 in this patent.
GKLIKQGKPV

SEQ ID NO: 486 - protein, 10 aa, mycoplasma genitalium
  Sequence located in MG420 at 215-224 and may interact with Sequence 485 in this patent.
DGLSLLDQLA

SEQ ID NO: 487 - protein, 10 aa, mycoplasma genitalium
  Sequence located in MG180 at 54-63 and may interact with Sequence 488 in this patent.
GDSGSGKSTL

SEQ ID NO: 488 - protein, 10 aa, mycoplasma genitalium
  Sequence located in MG208 at 70-79 and may interact with Sequence 487 in this patent.
TIGPGSFTGQ

SEQ ID NO: 489 - protein, 10 aa, mycoplasma genitalium
  Sequence located in MG180 at 53-62 and may interact with Sequence 490 in this patent.
IGDSGSGKST

SEQ ID NO: 490 - protein, 10 aa, mycoplasma genitalium
  Sequence located in MG321 at 410-419 and may interact with Sequence 489 in this patent.
GGLSASAVPY

SEQ ID NO: 491 - protein, 10 aa, mycoplasma genitalium
  Sequence located in MG181 at 156-165 and may interact with Sequence 492 in this patent.
VKGIDSLASM

SEQ ID NO: 492 - protein, 10 aa, mycoplasma genitalium
  Sequence located in MG307 at 649-658 and may interact with Sequence 491 in this patent.
HLPYVRKSGH

SEQ ID NO: 493 - protein, 10 aa, mycoplasma genitalium
  Sequence located in MG182 at 90-99 and may interact with Sequence 494 in this patent.
KTLVLVNDSF

SEQ ID NO: 494 - protein, 10 aa, mycoplasma genitalium
  Sequence located in MG341 at 706-715 and may interact with Sequence 493 in this patent.
LREDEHIIAE

SEQ ID NO: 495 - protein, 10 aa, mycoplasma genitalium
  Sequence located in MG182 at 45-54 and may interact with Sequence 496 in this patent.
VIGSGRTDKG

SEQ ID NO: 496 - protein, 10 aa, mycoplasma genitalium
  Sequence located in MG369 at 365-374 and may interact with Sequence 495 in this patent.
DDTGAPSVFS

SEQ ID NO: 497 - protein, 10 aa, mycoplasma genitalium
  Sequence located in MG187 at 286-295 and may interact with Sequence 498 in this patent.
LKQLKRLIKV

SEQ ID NO: 498 - protein, 10 aa, mycoplasma genitalium
  Sequence located in MG231 at 139-148 and may interact with Sequence 497 in this patent.
NFNKALKLLK

SEQ ID NO: 499 - protein, 10 aa, mycoplasma genitalium
  Sequence located in MG187 at 78-87 and may interact with Sequence 500 in this patent.
ERKLGLVFQN

SEQ ID NO: 500 - protein, 10 aa, mycoplasma genitalium
  Sequence located in MG437 at 78-87 and may interact with Sequence 499 in this patent.
ILKNKPKLSL

SEQ ID NO: 501 - protein, 10 aa, mycoplasma genitalium
  Sequence located in MG188 at 255-264 and may interact with Sequence 502 in this patent.
ENKPEEAVAN

SEQ ID NO: 502 - protein, 10 aa, mycoplasma genitalium
  Sequence located in MG345 at 643-652 and may interact with Sequence 501 in this patent.
IRNSLLRFIL

SEQ ID NO: 503 - protein, 10 aa, mycoplasma genitalium
  Sequence located in MG189 at 104-113 and may interact with Sequence 504 in this patent.
RVLSDGKYLN

SEQ ID NO: 504 - protein, 10 aa, mycoplasma genitalium
  Sequence located in MG386 at 356-365 and may interact with Sequence 503 in this patent.
TNETIPLIEI

SEQ ID NO: 505 - protein, 10 aa, mycoplasma genitalium
  Sequence located in MG191 at 962-971 and may interact with Sequence 506 in this patent.
NAALLHTYGF

SEQ ID NO: 506 - protein, 10 aa, mycoplasma genitalium
  Sequence located in MG321 at 605-614 and may interact with Sequence 505 in this patent.
VSSKQVSVTK

SEQ ID NO: 507 - protein, 10 aa, mycoplasma genitalium
  Sequence located in MG191 at 1411-1420 and may interact with Sequence 508 in this patent.
IINRTGISNA

SEQ ID NO: 508 - protein, 10 aa, mycoplasma genitalium
  Sequence located in MG360 at 60-69 and may interact with Sequence 507 in this patent.
NYVARSYGIR

SEQ ID NO: 509 - protein, 10 aa, mycoplasma genitalium
  Sequence located in MG191 at 1395-1404 and may interact with Sequence 510 in this patent.
KKVDVLTKAV

SEQ ID NO: 510 - protein, 10 aa, mycoplasma genitalium
  Sequence located in MG429 at 436-445 and may interact with Sequence 509 in this patent.
DCLGKHVDFF

SEQ ID NO: 511 - protein, 10 aa, mycoplasma genitalium
  Sequence located in MG191 at 1396-1405 and may interact with Sequence 512 in this patent.
KVDVLTKAVG

SEQ ID NO: 512 - protein, 10 aa, mycoplasma genitalium
  Sequence located in MG429 at 435-444 and may interact with Sequence 511 in this patent.
ADCLGKHVDF

SEQ ID NO: 513 - protein, 10 aa, mycoplasma genitalium
  Sequence located in MG191 at 1397-1406 and may interact with Sequence 514 in this patent.
VDVLTKAVGS

SEQ ID NO: 514 - protein, 10 aa, mycoplasma genitalium
  Sequence located in MG429 at 434-443 and may interact with Sequence 513 in this patent.
AADCLGKHVD

SEQ ID NO: 515 - protein, 10 aa, mycoplasma genitalium
  Sequence located in MG191 at 14-23 and may interact with Sequence 516 in this patent.
AFLTAALTLG

SEQ ID NO: 516 - protein, 10 aa, mycoplasma genitalium
  Sequence located in MG456 at 309-318 and may interact with Sequence 515 in this patent.
PESKSSSQKS

SEQ ID NO: 517 - protein, 10 aa, mycoplasma genitalium
  Sequence located in MG192 at 409-418 and may interact with Sequence 518 in this patent.
SSSSSSSSSS

SEQ ID NO: 518 - protein, 10 aa, mycoplasma genitalium
  Sequence located in MG338 at 358-367 and may interact with Sequence 517 in this patent.
TTTTTTTGGT

SEQ ID NO: 519 - protein, 10 aa, mycoplasma genitalium
  Sequence located in MG192 at 409-418 and may interact with Sequence 520 in this patent.
SSSSSSSSSS

SEQ ID NO: 520 - protein, 10 aa, mycoplasma genitalium
  Sequence located in MG338 at 357-366 and may interact with Sequence 519 in this patent.
TTTTTTTTGG

SEQ ID NO: 521 - protein, 10 aa, mycoplasma genitalium
  Sequence located in MG192 at 409-418 and may interact with Sequence 522 in this patent.
SSSSSSSSSS

SEQ ID NO: 522 - protein, 10 aa, mycoplasma genitalium
  Sequence located in MG338 at 356-365 and may interact with Sequence 521 in this patent.
TTTTTTTTTG

SEQ ID NO: 523 - protein, 10 aa, mycoplasma genitalium
  Sequence located in MG192 at 409-418 and may interact with Sequence 524 in this patent.
SSSSSSSSSS

SEQ ID NO: 524 - protein, 10 aa, mycoplasma genitalium
  Sequence located in MG338 at 355-364 and may interact with Sequence 523 in this patent.
TTTTTTTTTT

SEQ ID NO: 525 - protein, 10 aa, mycoplasma genitalium
  Sequence located in MG192 at 409-418 and may interact with Sequence 526 in this patent.
SSSSSSSSSS

SEQ ID NO: 526 - protein, 10 aa, mycoplasma genitalium
  Sequence located in MG338 at 354-363 and may interact with Sequence 525 in this patent.
TTTTTTTTTT

SEQ ID NO: 527 - protein, 10 aa, mycoplasma genitalium
  Sequence located in MG192 at 410-419 and may interact with Sequence 528 in this patent.
SSSSSSSSSS

SEQ ID NO: 528 - protein, 10 aa, mycoplasma genitalium
  Sequence located in MG338 at 358-367 and may interact with Sequence 527 in this patent.
TTTTTTTGGT

SEQ ID NO: 529 - protein, 10 aa, mycoplasma genitalium
  Sequence located in MG192 at 410-419 and may interact with Sequence 530 in this patent.
SSSSSSSSSS

SEQ ID NO: 530 - protein, 10 aa, mycoplasma genitalium
  Sequence located in MG338 at 357-366 and may interact with Sequence 529 in this patent.
TTTTTTTTGG

SEQ ID NO: 531 - protein, 10 aa, mycoplasma genitalium
  Sequence located in MG192 at 410-419 and may interact with Sequence 532 in this patent.
SSSSSSSSSS

SEQ ID NO: 532 - protein, 10 aa, mycoplasma genitalium
  Sequence located in MG338 at 356-365 and may interact with Sequence 531 in this patent.
TTTTTTTTTG

SEQ ID NO: 533 - protein, 10 aa, mycoplasma genitalium
  Sequence located in MG192 at 410-419 and may interact with Sequence 534 in this patent.
SSSSSSSSSS

SEQ ID NO: 534 - protein, 10 aa, mycoplasma genitalium
  Sequence located in MG338 at 355-364 and may interact with Sequence 533 in this patent.
TTTTTTTTTT

SEQ ID NO: 535 - protein, 10 aa, mycoplasma genitalium
  Sequence located in MG192 at 410-419 and may interact with Sequence 536 in this patent.
SSSSSSSSSS

SEQ ID NO: 536 - protein, 10 aa, mycoplasma genitalium
  Sequence located in MG338 at 354-363 and may interact with Sequence 535 in this patent.
TTTTTTTTTT

SEQ ID NO: 537 - protein, 10 aa, mycoplasma genitalium
  Sequence located in MG192 at 411-420 and may interact with Sequence 538 in this patent.
SSSSSSSSST

SEQ ID NO: 538 - protein, 10 aa, mycoplasma genitalium
  Sequence located in MG338 at 359-368 and may interact with Sequence 537 in this patent.
TTTTTTGGTS

SEQ ID NO: 539 - protein, 10 aa, mycoplasma genitalium
  Sequence located in MG192 at 411-420 and may interact with Sequence 540 in this patent.
SSSSSSSSST

SEQ ID NO: 540 - protein, 10 aa, mycoplasma genitalium
  Sequence located in MG338 at 357-366 and may interact with Sequence 539 in this patent.
TTTTTTTTGG

SEQ ID NO: 541 - protein, 10 aa, mycoplasma genitalium
  Sequence located in MG192 at 411-420 and may interact with Sequence 542 in this patent.
SSSSSSSSST

SEQ ID NO: 542 - protein, 10 aa, mycoplasma genitalium
  Sequence located in MG338 at 356-365 and may interact with Sequence 541 in this patent.
TTTTTTTTTG

SEQ ID NO: 543 - protein, 10 aa, mycoplasma genitalium
  Sequence located in MG192 at 410-419 and may interact with Sequence 544 in this patent.
SSSSSSSSSS

SEQ ID NO: 544 - protein, 10 aa, mycoplasma genitalium
  Sequence located in MG338 at 358-367 and may interact with Sequence 543 in this patent.
TTTTTTTGGT

SEQ ID NO: 545 - protein, 10 aa, mycoplasma genitalium
  Sequence located in MG192 at 410-419 and may interact with Sequence 546 in this patent.
SSSSSSSSSS

SEQ ID NO: 546 - protein, 10 aa, mycoplasma genitalium
  Sequence located in MG338 at 357-366 and may interact with Sequence 545 in this patent.
TTTTTTTTGG

SEQ ID NO: 547 - protein, 10 aa, mycoplasma genitalium
  Sequence located in MG192 at 410-419 and may interact with Sequence 548 in this patent.
SSSSSSSSSS

SEQ ID NO: 548 - protein, 10 aa, mycoplasma genitalium
  Sequence located in MG338 at 356-365 and may interact with Sequence 547 in this patent.
TTTTTTTTTG

SEQ ID NO: 549 - protein, 10 aa, mycoplasma genitalium
  Sequence located in MG192 at 410-419 and may interact with Sequence 550 in this patent.
SSSSSSSSSS

SEQ ID NO: 550 - protein, 10 aa, mycoplasma genitalium
  Sequence located in MG338 at 355-364 and may interact with Sequence 549 in this patent.
TTTTTTTTTT

SEQ ID NO: 551 - protein, 10 aa, mycoplasma genitalium
  Sequence located in MG192 at 410-419 and may interact with Sequence 552 in this patent.
SSSSSSSSSS

SEQ ID NO: 552 - protein, 10 aa, mycoplasma genitalium
  Sequence located in MG338 at 354-363 and may interact with Sequence 551 in this patent.
TTTTTTTTTT

SEQ ID NO: 553 - protein, 10 aa, mycoplasma genitalium
  Sequence located in MG192 at 409-418 and may interact with Sequence 554 in this patent.
SSSSSSSSSS

SEQ ID NO: 554 - protein, 10 aa, mycoplasma genitalium
  Sequence located in MG338 at 358-367 and may interact with Sequence 553 in this patent.
TTTTTTTGGT

SEQ ID NO: 555 - protein, 10 aa, mycoplasma genitalium
  Sequence located in MG192 at 409-418 and may interact with Sequence 556 in this patent.
SSSSSSSSSS

SEQ ID NO: 556 - protein, 10 aa, mycoplasma genitalium
  Sequence located in MG338 at 357-366 and may interact with Sequence 555 in this patent.
TTTTTTTTGG

SEQ ID NO: 557 - protein, 10 aa, mycoplasma genitalium
  Sequence located in MG192 at 409-418 and may interact with Sequence 558 in this patent.
SSSSSSSSSS

SEQ ID NO: 558 - protein, 10 aa, mycoplasma genitalium
  Sequence located in MG338 at 356-365 and may interact with Sequence 557 in this patent.
TTTTTTTTTG

SEQ ID NO: 559 - protein, 10 aa, mycoplasma genitalium
  Sequence located in MG192 at 409-418 and may interact with Sequence 560 in this patent.
SSSSSSSSSS

SEQ ID NO: 560 - protein, 10 aa, mycoplasma genitalium
  Sequence located in MG338 at 355-364 and may interact with Sequence 559 in this patent.
TTTTTTTTTT

SEQ ID NO: 561 - protein, 10 aa, mycoplasma genitalium
  Sequence located in MG192 at 409-418 and may interact with Sequence 562 in this patent.
SSSSSSSSSS

SEQ ID NO: 562 - protein, 10 aa, mycoplasma genitalium
  Sequence located in MG338 at 354-363 and may interact with Sequence 561 in this patent.
TTTTTTTTTT

SEQ ID NO: 563 - protein, 10 aa, mycoplasma genitalium
  Sequence located in MG192 at 716-725 and may interact with Sequence 564 in this patent.
SSLKPSHDGK

SEQ ID NO: 564 - protein, 10 aa, mycoplasma genitalium
  Sequence located in MG355 at 203-212 and may interact with Sequence 563 in this patent.
LTIMRGLKTR

SEQ ID NO: 565 - protein, 10 aa, mycoplasma genitalium
  Sequence located in MG192 at 407-416 and may interact with Sequence 566 in this patent.
YDSSSSSSSS

SEQ ID NO: 566 - protein, 10 aa, mycoplasma genitalium
  Sequence located in MG362 at 39-48 and may interact with Sequence 565 in this patent.
IVAAGAAGAT

SEQ ID NO: 567 - protein, 10 aa, mycoplasma genitalium
  Sequence located in MG192 at 406-415 and may interact with Sequence 568 in this patent.
GYDSSSSSSS

SEQ ID NO: 568 - protein, 10 aa, mycoplasma genitalium
  Sequence located in MG362 at 38-47 and may interact with Sequence 567 in this patent.
PIVAAGAAGA

SEQ ID NO: 569 - protein, 10 aa, mycoplasma genitalium
  Sequence located in MG192 at 236-245 and may interact with Sequence 570 in this patent.
DSSGWSSTEE

SEQ ID NO: 570 - protein, 10 aa, mycoplasma genitalium
  Sequence located in MG383 at 180-189 and may interact with Sequence 569 in this patent.
ITRAPTASLF

SEQ ID NO: 571 - protein, 10 aa, mycoplasma genitalium
  Sequence located in MG194 at 106-115 and may interact with Sequence 572 in this patent.
LYQVIDNLVE

SEQ ID NO: 572 - protein, 10 aa, mycoplasma genitalium
  Sequence located in MG194 at 106-115 and may interact with Sequence 571 in this patent.
LYQVIDNLVE

SEQ ID NO: 573 - protein, 10 aa, mycoplasma genitalium
  Sequence located in MG194 at 181-190 and may interact with Sequence 574 in this patent.
TNNPDIRVVS

SEQ ID NO: 574 - protein, 10 aa, mycoplasma genitalium
  Sequence located in MG194 at 181-190 and may interact with Sequence 573 in this patent.
TNNPDIRVVS

SEQ ID NO: 575 - protein, 10 aa, mycoplasma genitalium
  Sequence located in MG194 at 182-191 and may interact with Sequence 576 in this patent.
NNPDIRVVSL

SEQ ID NO: 576 - protein, 10 aa, mycoplasma genitalium
  Sequence located in MG194 at 180-189 and may interact with Sequence 575 in this patent.
KTNNPDIRVV

SEQ ID NO: 577 - protein, 10 aa, mycoplasma genitalium
  Sequence located in MG194 at 168-177 and may interact with Sequence 578 in this patent.
TATTLKAVRT

SEQ ID NO: 578 - protein, 10 aa, mycoplasma genitalium
  Sequence located in MG322 at 144-153 and may interact with Sequence 577 in this patent.
RGGSKLGNTS

SEQ ID NO: 579 - protein, 10 aa, mycoplasma genitalium
  Sequence located in MG194 at 244-253 and may interact with Sequence 580 in this patent.
RFRLSHFPFT

SEQ ID NO: 580 - protein, 10 aa, mycoplasma genitalium
  Sequence located in MG340 at 34-43 and may interact with Sequence 579 in this patent.
SEGEVTKAET

SEQ ID NO: 581 - protein, 10 aa, mycoplasma genitalium
  Sequence located in MG196 at 31-40 and may interact with Sequence 582 in this patent.
ENSTNLGIVK

SEQ ID NO: 582 - protein, 10 aa, mycoplasma genitalium
  Sequence located in MG209 at 69-78 and may interact with Sequence 581 in this patent.
FITSVEPYNL

SEQ ID NO: 583 - protein, 10 aa, mycoplasma genitalium
  Sequence located in MG196 at 146-155 and may interact with Sequence 584 in this patent.
VFDDFYQLVK

SEQ ID NO: 584 - protein, 10 aa, mycoplasma genitalium
  Sequence located in MG252 at 82-91 and may interact with Sequence 583 in this patent.
LDQLVKVVEN

SEQ ID NO: 585 - protein, 10 aa, mycoplasma genitalium
  Sequence located in MG198 at 45-54 and may interact with Sequence 586 in this patent.
AYRDRRQKKR

SEQ ID NO: 586 - protein, 10 aa, mycoplasma genitalium
  Sequence located in MG309 at 11-20 and may interact with Sequence 585 in this patent.
PLLLSSIAVS

SEQ ID NO: 587 - protein, 10 aa, mycoplasma genitalium
  Sequence located in MG198 at 89-98 and may interact with Sequence 588 in this patent.
INRKVLSELA

SEQ ID NO: 588 - protein, 10 aa, mycoplasma genitalium
  Sequence located in MG463 at 8-17 and may interact with Sequence 587 in this patent.
RKLGQNFTVN

SEQ ID NO: 589 - protein, 10 aa, mycoplasma genitalium
  Sequence located in MG200 at 484-493 and may interact with Sequence 590 in this patent.
PLVAYNGGII

SEQ ID NO: 590 - protein, 10 aa, mycoplasma genitalium
  Sequence located in MG392 at 168-177 and may interact with Sequence 589 in this patent.
GKNGVITTDD

SEQ ID NO: 591 - protein, 10 aa, mycoplasma genitalium
  Sequence located in MG200 at 483-492 and may interact with Sequence 592 in this patent.
DPLVAYNGGI

SEQ ID NO: 592 - protein, 10 aa, mycoplasma genitalium
  Sequence located in MG392 at 167-176 and may interact with Sequence 591 in this patent.
VGKNGVITTD

SEQ ID NO: 593 - protein, 10 aa, mycoplasma genitalium
  Sequence located in MG203 at 539-548 and may interact with Sequence 594 in this patent.
ETKDRKTVKY

SEQ ID NO: 594 - protein, 10 aa, mycoplasma genitalium
  Sequence located in MG406 at 8-17 and may interact with Sequence 593 in this patent.
VLNSLSVLRL

SEQ ID NO: 595 - protein, 10 aa, mycoplasma genitalium
  Sequence located in MG204 at 576-585 and may interact with Sequence 596 in this patent.
VSKKPILLEY

SEQ ID NO: 596 - protein, 10 aa, mycoplasma genitalium
  Sequence located in MG468.1 at 139-148 and may interact with Sequence 595 in this patent.
IFQQYGLLRD

SEQ ID NO: 597 - protein, 10 aa, mycoplasma genitalium
  Sequence located in MG208 at 41-50 and may interact with Sequence 598 in this patent.
NLTELAVYYL

SEQ ID NO: 598 - protein, 10 aa, mycoplasma genitalium
  Sequence located in MG377 at 90-99 and may interact with Sequence 597 in this patent.
VEGLKRDVVK

SEQ ID NO: 599 - protein, 10 aa, mycoplasma genitalium
  Sequence located in MG209 at 14-23 and may interact with Sequence 600 in this patent.
DSLLASLLNL

SEQ ID NO: 600 - protein, 10 aa, mycoplasma genitalium
  Sequence located in MG458 at 12-21 and may interact with Sequence 599 in this patent.
QIEEGCQKAV

SEQ ID NO: 601 - protein, 10 aa, mycoplasma genitalium
  Sequence located in MG212 at 240-249 and may interact with Sequence 602 in this patent.
FNQIDSNNLA

SEQ ID NO: 602 - protein, 10 aa, mycoplasma genitalium
  Sequence located in MG265 at 129-138 and may interact with Sequence 601 in this patent.
SQIVGIDLIE

SEQ ID NO: 603 - protein, 10 aa, mycoplasma genitalium
  Sequence located in MG215 at 278-287 and may interact with Sequence 604 in this patent.
LVVNNANSQV

SEQ ID NO: 604 - protein, 10 aa, mycoplasma genitalium
  Sequence located in MG428 at 29-38 and may interact with Sequence 603 in this patent.
EDDIISIALH

SEQ ID NO: 605 - protein, 10 aa, mycoplasma genitalium
  Sequence located in MG217 at 209-218 and may interact with Sequence 606 in this patent.
FDSYNFRLNS

SEQ ID NO: 606 - protein, 10 aa, mycoplasma genitalium
  Sequence located in MG302 at 180-189 and may interact with Sequence 605 in this patent.
TVKSEIIRIK

SEQ ID NO: 607 - protein, 10 aa, mycoplasma genitalium
  Sequence located in MG218 at 1136-1145 and may interact with Sequence 608 in this patent.
EANAKKAFLI

SEQ ID NO: 608 - protein, 10 aa, mycoplasma genitalium
  Sequence located in MG336 at 370-379 and may interact with Sequence 607 in this patent.
NKESFLRVSL

SEQ ID NO: 609 - protein, 10 aa, mycoplasma genitalium
  Sequence located in MG218 at 1323-1332 and may interact with Sequence 610 in this patent.
ETERKRAVLE

SEQ ID NO: 610 - protein, 10 aa, mycoplasma genitalium
  Sequence located in MG340 at 212-221 and may interact with Sequence 609 in this patent.
LKNSSLPFSL

SEQ ID NO: 611 - protein, 10 aa, mycoplasma genitalium
  Sequence located in MG222 at 91-100 and may interact with Sequence 612 in this patent.
LSITKFDGIL

SEQ ID NO: 612 - protein, 10 aa, mycoplasma genitalium
  Sequence located in MG303 at 209-218 and may interact with Sequence 611 in this patent.
KNPIELSDGQ

SEQ ID NO: 613 - protein, 10 aa, mycoplasma genitalium
  Sequence located in MG226 at 95-104 and may interact with Sequence 614 in this patent.
KANNNIIIGL

SEQ ID NO: 614 - protein, 10 aa, mycoplasma genitalium
  Sequence located in MG226 at 95-104 and may interact with Sequence 613 in this patent.
KANNNIIIGL

SEQ ID NO: 615 - protein, 10 aa, mycoplasma genitalium
  Sequence located in MG226 at 400-409 and may interact with Sequence 616 in this patent.
ISFALVSVLL

SEQ ID NO: 616 - protein, 10 aa, mycoplasma genitalium
  Sequence located in MG231 at 269-278 and may interact with Sequence 615 in this patent.
DTKRENADEK

SEQ ID NO: 617 - protein, 10 aa, mycoplasma genitalium
  Sequence located in MG226 at 116-125 and may interact with Sequence 618 in this patent.
TFISLFGLKI

SEQ ID NO: 618 - protein, 10 aa, mycoplasma genitalium
  Sequence located in MG352 at 68-77 and may interact with Sequence 617 in this patent.
DFEAKQTNKS

SEQ ID NO: 619 - protein, 10 aa, mycoplasma genitalium
  Sequence located in MG230 at 37-46 and may interact with Sequence 620 in this patent.
EKLGFQHKRI

SEQ ID NO: 620 - protein, 10 aa, mycoplasma genitalium
  Sequence located in MG255.1 at 7-16 and may interact with Sequence 619 in this patent.
NSLVLKPELF

SEQ ID NO: 621 - protein, 10 aa, mycoplasma genitalium
  Sequence located in MG237 at 211-220 and may interact with Sequence 622 in this patent.
IKKVSQLFLK

SEQ ID NO: 622 - protein, 10 aa, mycoplasma genitalium
  Sequence located in MG384 at 313-322 and may interact with Sequence 621 in this patent.
LKKELGNLLD

SEQ ID NO: 623 - protein, 10 aa, mycoplasma genitalium
  Sequence located in MG242 at 424-433 and may interact with Sequence 624 in this patent.
NDFYQFEKDI

SEQ ID NO: 624 - protein, 10 aa, mycoplasma genitalium
  Sequence located in MG292 at 247-256 and may interact with Sequence 623 in this patent.
DIFLKLIKII

SEQ ID NO: 625 - protein, 10 aa, mycoplasma genitalium
  Sequence located in MG251 at 384-393 and may interact with Sequence 626 in this patent.
CFETTGSIGK

SEQ ID NO: 626 - protein, 10 aa, mycoplasma genitalium
  Sequence located in MG309 at 847-856 and may interact with Sequence 625 in this patent.
AKLSRTTNSF

SEQ ID NO: 627 - protein, 10 aa, mycoplasma genitalium
  Sequence located in MG253 at 200-209 and may interact with Sequence 628 in this patent.
IDYCFKNELT

SEQ ID NO: 628 - protein, 10 aa, mycoplasma genitalium
  Sequence located in MG356 at 223-232 and may interact with Sequence 627 in this patent.
RKLVFETVIY

SEQ ID NO: 629 - protein, 10 aa, mycoplasma genitalium
  Sequence located in MG260 at 247-256 and may interact with Sequence 630 in this patent.
GETKDTTKSD

SEQ ID NO: 630 - protein, 10 aa, mycoplasma genitalium
  Sequence located in MG321 at 689-698 and may interact with Sequence 629 in this patent.
IGLSSVLGFS

SEQ ID NO: 631 - protein, 10 aa, mycoplasma genitalium
  Sequence located in MG260 at 138-147 and may interact with Sequence 632 in this patent.
SRIGGIRDDG

SEQ ID NO: 632 - protein, 10 aa, mycoplasma genitalium
  Sequence located in MG329 at 27-36 and may interact with Sequence 631 in this patent.
AIVSDTPNTT

SEQ ID NO: 633 - protein, 10 aa, mycoplasma genitalium
  Sequence located in MG260 at 237-246 and may interact with Sequence 634 in this patent.
SSSSSTTSTT

SEQ ID NO: 634 - protein, 10 aa, mycoplasma genitalium
  Sequence located in MG338 at 360-369 and may interact with Sequence 633 in this patent.
TTTTTGGTSS

SEQ ID NO: 635 - protein, 10 aa, mycoplasma genitalium
  Sequence located in MG260 at 236-245 and may interact with Sequence 636 in this patent.
SSSSSSTTST

SEQ ID NO: 636 - protein, 10 aa, mycoplasma genitalium
  Sequence located in MG338 at 359-368 and may interact with Sequence 635 in this patent.
TTTTTTGGTS

SEQ ID NO: 637 - protein, 10 aa, mycoplasma genitalium
  Sequence located in MG260 at 235-244 and may interact with Sequence 638 in this patent.
RSSSSSSTTS

SEQ ID NO: 638 - protein, 10 aa, mycoplasma genitalium
  Sequence located in MG338 at 358-367 and may interact with Sequence 637 in this patent.
TTTTTTTGGT

SEQ ID NO: 639 - protein, 10 aa, mycoplasma genitalium
  Sequence located in MG260 at 202-211 and may interact with Sequence 640 in this patent.
IANDSETTKL

SEQ ID NO: 640 - protein, 10 aa, mycoplasma genitalium
  Sequence located in MG438 at 354-363 and may interact with Sequence 639 in this patent.
ELSSLTVIRD

SEQ ID NO: 641 - protein, 10 aa, mycoplasma genitalium
  Sequence located in MG260 at 56-65 and may interact with Sequence 642 in this patent.
VKKYNDTKDP

SEQ ID NO: 642 - protein, 10 aa, mycoplasma genitalium
  Sequence located in MG447 at 304-313 and may interact with Sequence 641 in this patent.
NLLVVVSLIG

SEQ ID NO: 643 - protein, 10 aa, mycoplasma genitalium
  Sequence located in MG264 at 157-166 and may interact with Sequence 644 in this patent.
PNCKIDTIFN

SEQ ID NO: 644 - protein, 10 aa, mycoplasma genitalium
  Sequence located in MG300 at 295-304 and may interact with Sequence 643 in this patent.
GITLDVSDKI

SEQ ID NO: 645 - protein, 10 aa, mycoplasma genitalium
  Sequence located in MG265 at 204-213 and may interact with Sequence 646 in this patent.
KAYGLKVLVD

SEQ ID NO: 646 - protein, 10 aa, mycoplasma genitalium
  Sequence located in MG437 at 44-53 and may interact with Sequence 645 in this patent.
INENFKAVRF

SEQ ID NO: 647 - protein, 10 aa, mycoplasma genitalium
  Sequence located in MG265 at 11-20 and may interact with Sequence 648 in this patent.
DGTLLSSNQI

SEQ ID NO: 648 - protein, 10 aa, mycoplasma genitalium
  Sequence located in MG453 at 58-67 and may interact with Sequence 647 in this patent.
VSSKKTAILD

SEQ ID NO: 649 - protein, 10 aa, mycoplasma genitalium
  Sequence located in MG266 at 643-652 and may interact with Sequence 650 in this patent.
FAYNLFLKNS

SEQ ID NO: 650 - protein, 10 aa, mycoplasma genitalium
  Sequence located in MG422 at 311-320 and may interact with Sequence 649 in this patent.
RILEKKIVSK

SEQ ID NO: 651 - protein, 10 aa, mycoplasma genitalium
  Sequence located in MG294 at 414-423 and may interact with Sequence 652 in this patent.
KKYTVAGQSN

SEQ ID NO: 652 - protein, 10 aa, mycoplasma genitalium
  Sequence located in MG447 at 249-258 and may interact with Sequence 651 in this patent.
LLVRYSSLGV

SEQ ID NO: 653 - protein, 10 aa, mycoplasma genitalium
  Sequence located in MG301 at 11-20 and may interact with Sequence 654 in this patent.
AINGFGRIGR

SEQ ID NO: 654 - protein, 10 aa, mycoplasma genitalium
  Sequence located in MG395 at 209-218 and may interact with Sequence 653 in this patent.
SNIPKTAYTA

SEQ ID NO: 655 - protein, 10 aa, mycoplasma genitalium
  Sequence located in MG301 at 66-75 and may interact with Sequence 656 in this patent.
KQNILQIDRK

SEQ ID NO: 656 - protein, 10 aa, mycoplasma genitalium
  Sequence located in MG414 at 851-860 and may interact with Sequence 655 in this patent.
LLVDKLNITL

SEQ ID NO: 657 - protein, 10 aa, mycoplasma genitalium
  Sequence located in MG301 at 73-82 and may interact with Sequence 658 in this patent.
DRKKVYVFSE

SEQ ID NO: 658 - protein, 10 aa, mycoplasma genitalium
  Sequence located in MG429 at 438-447 and may interact with Sequence 657 in this patent.
LGKHVDFFSI

SEQ ID NO: 659 - protein, 10 aa, mycoplasma genitalium
  Sequence located in MG301 at 283-292 and may interact with Sequence 660 in this patent.
SSDVVSSEYG

SEQ ID NO: 660 - protein, 10 aa, mycoplasma genitalium
  Sequence located in MG438 at 234-243 and may interact with Sequence 659 in this patent.
AGIDNTGFVA

SEQ ID NO: 661 - protein, 10 aa, mycoplasma genitalium
  Sequence located in MG303 at 34-43 and may interact with Sequence 662 in this patent.
LFSFYKKVDK

SEQ ID NO: 662 - protein, 10 aa, mycoplasma genitalium
  Sequence located in MG390 at 441-450 and may interact with Sequence 661 in this patent.
LVNLFIETKK

SEQ ID NO: 663 - protein, 10 aa, mycoplasma genitalium
  Sequence located in MG307 at 284-293 and may interact with Sequence 664 in this patent.
GKDGLNTAKT

SEQ ID NO: 664 - protein, 10 aa, mycoplasma genitalium
  Sequence located in MG339 at 175-184 and may interact with Sequence 663 in this patent.
GLRRIQSILP

SEQ ID NO: 665 - protein, 10 aa, mycoplasma genitalium
  Sequence located in MG307 at 504-513 and may interact with Sequence 666 in this patent.
GSYYLTKNSS

SEQ ID NO: 666 - protein, 10 aa, mycoplasma genitalium
  Sequence located in MG355 at 67-76 and may interact with Sequence 665 in this patent.
TAIVEGFVRR

SEQ ID NO: 667 - protein, 10 aa, mycoplasma genitalium
  Sequence located in MG307 at 564-573 and may interact with Sequence 668 in this patent.
LILVNALINT

SEQ ID NO: 668 - protein, 10 aa, mycoplasma genitalium
  Sequence located in MG414 at 495-504 and may interact with Sequence 667 in this patent.
RIDESIDKNE

SEQ ID NO: 669 - protein, 10 aa, mycoplasma genitalium
  Sequence located in MG307 at 124-133 and may interact with Sequence 670 in this patent.
FIQQDLLDKS

SEQ ID NO: 670 - protein, 10 aa, mycoplasma genitalium
  Sequence located in MG463 at 68-77 and may interact with Sequence 669 in this patent.
EYLLVEKILT

SEQ ID NO: 671 - protein, 10 aa, mycoplasma genitalium
  Sequence located in MG317 at 148-157 and may interact with Sequence 672 in this patent.
IDAGLPKIEV

SEQ ID NO: 672 - protein, 10 aa, mycoplasma genitalium
  Sequence located in MG439 at 105-114 and may interact with Sequence 671 in this patent.
NISAKGLYFD

SEQ ID NO: 673 - protein, 10 aa, mycoplasma genitalium
  Sequence located in MG321 at 591-600 and may interact with Sequence 674 in this patent.
RTASTSGSSS

SEQ ID NO: 674 - protein, 10 aa, mycoplasma genitalium
  Sequence located in MG321 at 591-600 and may interact with Sequence 673 in this patent.
RTASTSGSSS

SEQ ID NO: 675 - protein, 10 aa, mycoplasma genitalium
  Sequence located in MG321 at 205-214 and may interact with Sequence 676 in this patent.
GFETYILSSN

SEQ ID NO: 676 - protein, 10 aa, mycoplasma genitalium
  Sequence located in MG421 at 306-315 and may interact with Sequence 675 in this patent.
SKLSINQGAI

SEQ ID NO: 677 - protein, 10 aa, mycoplasma genitalium
  Sequence located in MG322 at 274-283 and may interact with Sequence 678 in this patent.
LFDGFEAIKK

SEQ ID NO: 678 - protein, 10 aa, mycoplasma genitalium
  Sequence located in MG322 at 274-283 and may interact with Sequence 677 in this patent.
LFDGFEAIKK

SEQ ID NO: 679 - protein, 10 aa, mycoplasma genitalium
  Sequence located in MG324 at 23-32 and may interact with Sequence 680 in this patent.
AILIGSDQNR

SEQ ID NO: 680 - protein, 10 aa, mycoplasma genitalium
  Sequence located in MG324 at 23-32 and may interact with Sequence 679 in this patent.
AILIGSDQNR

SEQ ID NO: 681 - protein, 10 aa, mycoplasma genitalium
  Sequence located in MG325 at 2-11 and may interact with Sequence 682 in this patent.
AVKRSTRLGC

SEQ ID NO: 682 - protein, 10 aa, mycoplasma genitalium
  Sequence located in MG468 at 1110-1119 and may interact with Sequence 681 in this patent.
AAQTSTSLNR

SEQ ID NO: 683 - protein, 10 aa, mycoplasma genitalium
  Sequence located in MG327 at 126-135 and may interact with Sequence 684 in this patent.
QTSFSVNKKR

SEQ ID NO: 684 - protein, 10 aa, mycoplasma genitalium
  Sequence located in MG469 at 136-145 and may interact with Sequence 683 in this patent.
PLFIYGETGL

SEQ ID NO: 685 - protein, 10 aa, mycoplasma genitalium
  Sequence located in MG330 at 113-122 and may interact with Sequence 686 in this patent.
NKNIVMDGRD

SEQ ID NO: 686 - protein, 10 aa, mycoplasma genitalium
  Sequence located in MG393 at 3-12 and may interact with Sequence 685 in this patent.
ITPIHDNVLV

SEQ ID NO: 687 - protein, 10 aa, mycoplasma genitalium
  Sequence located in MG330 at 114-123 and may interact with Sequence 688 in this patent.
KNIVMDGRDI

SEQ ID NO: 688 - protein, 10 aa, mycoplasma genitalium
  Sequence located in MG393 at 2-11 and may interact with Sequence 687 in this patent.
NITPIHDNVL

SEQ ID NO: 689 - protein, 10 aa, mycoplasma genitalium
  Sequence located in MG330 at 82-91 and may interact with Sequence 690 in this patent.
TTQSVANIAS

SEQ ID NO: 690 - protein, 10 aa, mycoplasma genitalium
  Sequence located in MG448 at 116-125 and may interact with Sequence 689 in this patent.
GGLRYCINSA

SEQ ID NO: 691 - protein, 10 aa, mycoplasma genitalium
  Sequence located in MG334 at 228-237 and may interact with Sequence 692 in this patent.
DVCLLVNPKD

SEQ ID NO: 692 - protein, 10 aa, mycoplasma genitalium
  Sequence located in MG360 at 340-349 and may interact with Sequence 691 in this patent.
INTEKNVRLI

SEQ ID NO: 693 - protein, 10 aa, mycoplasma genitalium
  Sequence located in MG335.2 at 210-219 and may interact with Sequence 694 in this patent.
FIDVIGTNYF

SEQ ID NO: 694 - protein, 10 aa, mycoplasma genitalium
  Sequence located in MG421 at 683-692 and may interact with Sequence 693 in this patent.
EIIGANNIDK

SEQ ID NO: 695 - protein, 10 aa, mycoplasma genitalium
  Sequence located in MG338 at 485-494 and may interact with Sequence 696 in this patent.
VYVRGDDAIY

SEQ ID NO: 696 - protein, 10 aa, mycoplasma genitalium
  Sequence located in MG357 at 240-249 and may interact with Sequence 695 in this patent.
DIDPAIVSYI

SEQ ID NO: 697 - protein, 10 aa, mycoplasma genitalium
  Sequence located in MG338 at 484-493 and may interact with Sequence 698 in this patent.
PVYVRGDDAI

SEQ ID NO: 698 - protein, 10 aa, mycoplasma genitalium
  Sequence located in MG357 at 239-248 and may interact with Sequence 697 in this patent.
GDIDPAIVSY

SEQ ID NO: 699 - protein, 10 aa, mycoplasma genitalium
  Sequence located in MG338 at 171-180 and may interact with Sequence 700 in this patent.
NSSDTSVATT

SEQ ID NO: 700 - protein, 10 aa, mycoplasma genitalium
  Sequence located in MG412 at 68-77 and may interact with Sequence 699 in this patent.
GGSNAGIRAI

SEQ ID NO: 701 - protein, 10 aa, mycoplasma genitalium
  Sequence located in MG342 at 45-54 and may interact with Sequence 702 in this patent.
FYSVDLEATN

SEQ ID NO: 702 - protein, 10 aa, mycoplasma genitalium
  Sequence located in MG392 at 220-229 and may interact with Sequence 701 in this patent.
VSSLKINTIK

SEQ ID NO: 703 - protein, 10 aa, mycoplasma genitalium
  Sequence located in MG351 at 34-43 and may interact with Sequence 704 in this patent.
ILFGSESYPQ

SEQ ID NO: 704 - protein, 10 aa, mycoplasma genitalium
  Sequence located in MG459 at 3-12 and may interact with Sequence 703 in this patent.
LRVGLGSKKY

SEQ ID NO: 705 - protein, 10 aa, mycoplasma genitalium
  Sequence located in MG354 at 9-18 and may interact with Sequence 706 in this patent.
QLISFFNQAC

SEQ ID NO: 706 - protein, 10 aa, mycoplasma genitalium
  Sequence located in MG442 at 235-244 and may interact with Sequence 705 in this patent.
RGLIKKANEL

SEQ ID NO: 707 - protein, 10 aa, mycoplasma genitalium
  Sequence located in MG360 at 347-356 and may interact with Sequence 708 in this patent.
RLIGISFFDL

SEQ ID NO: 708 - protein, 10 aa, mycoplasma genitalium
  Sequence located in MG386 at 563-572 and may interact with Sequence 707 in this patent.
KIKETNSDES

SEQ ID NO: 709 - protein, 10 aa, mycoplasma genitalium
  Sequence located in MG365 at 8-17 and may interact with Sequence 710 in this patent.
GTSTLSKKCL

SEQ ID NO: 710 - protein, 10 aa, mycoplasma genitalium
  Sequence located in MG457 at 273-282 and may interact with Sequence 709 in this patent.
PGTGKTLLAK

SEQ ID NO: 711 - protein, 10 aa, mycoplasma genitalium
  Sequence located in MG366 at 440-449 and may interact with Sequence 712 in this patent.
KKVYFDFETI

SEQ ID NO: 712 - protein, 10 aa, mycoplasma genitalium
  Sequence located in MG407 at 429-438 and may interact with Sequence 711 in this patent.
LLYIEIELGD

SEQ ID NO: 713 - protein, 10 aa, mycoplasma genitalium
  Sequence located in MG366 at 53-62 and may interact with Sequence 714 in this patent.
PVFELDVLEL

SEQ ID NO: 714 - protein, 10 aa, mycoplasma genitalium
  Sequence located in MG423 at 113-122 and may interact with Sequence 713 in this patent.
RDKLEIHELK

SEQ ID NO: 715 - protein, 10 aa, mycoplasma genitalium
  Sequence located in MG376 at 17-26 and may interact with Sequence 716 in this patent.
SHKKTGFLVT

SEQ ID NO: 716 - protein, 10 aa, mycoplasma genitalium
  Sequence located in MG376 at 17-26 and may interact with Sequence 715 in this patent.
SHKKTGFLVT

SEQ ID NO: 717 - protein, 10 aa, mycoplasma genitalium
  Sequence located in MG379 at 152-161 and may interact with Sequence 718 in this patent.
GTYLRSKTYC

SEQ ID NO: 718 - protein, 10 aa, mycoplasma genitalium
  Sequence located in MG421 at 504-513 and may interact with Sequence 717 in this patent.
RIRLATQIGS

SEQ ID NO: 719 - protein, 10 aa, mycoplasma genitalium
  Sequence located in MG381 at 180-189 and may interact with Sequence 720 in this patent.
FGEYLSSFHL

SEQ ID NO: 720 - protein, 10 aa, mycoplasma genitalium
  Sequence located in MG451 at 286-295 and may interact with Sequence 719 in this patent.
EVERGQVLAK

SEQ ID NO: 721 - protein, 10 aa, mycoplasma genitalium
  Sequence located in MG390 at 388-397 and may interact with Sequence 722 in this patent.
AILGLGVIGI

SEQ ID NO: 722 - protein, 10 aa, mycoplasma genitalium
  Sequence located in MG468 at 267-276 and may interact with Sequence 721 in this patent.
SDETKADNTD

SEQ ID NO: 723 - protein, 10 aa, mycoplasma genitalium
  Sequence located in MG390 at 387-396 and may interact with Sequence 724 in this patent.
FAILGLGVIG

SEQ ID NO: 724 - protein, 10 aa, mycoplasma genitalium
  Sequence located in MG468 at 266-275 and may interact with Sequence 723 in this patent.
KSDETKADNT

SEQ ID NO: 725 - protein, 10 aa, mycoplasma genitalium
  Sequence located in MG391 at 392-401 and may interact with Sequence 726 in this patent.
SARGAGSSRA

SEQ ID NO: 726 - protein, 10 aa, mycoplasma genitalium
  Sequence located in MG391 at 392-401 and may interact with Sequence 725 in this patent.
SARGAGSSRA

SEQ ID NO: 727 - protein, 10 aa, mycoplasma genitalium
  Sequence located in MG393 at 85-94 and may interact with Sequence 728 in this patent.
GNKYKIIGFE

SEQ ID NO: 728 - protein, 10 aa, mycoplasma genitalium
  Sequence located in MG394 at 359-368 and may interact with Sequence 727 in this patent.
FKTNDFIFVA

SEQ ID NO: 729 - protein, 10 aa, mycoplasma genitalium
  Sequence located in MG395 at 456-465 and may interact with Sequence 730 in this patent.
SEKNKTRTTN

SEQ ID NO: 730 - protein, 10 aa, mycoplasma genitalium
  Sequence located in MG398 at 5-14 and may interact with Sequence 729 in this patent.
RFLVLSPSGI

SEQ ID NO: 731 - protein, 10 aa, mycoplasma genitalium
  Sequence located in MG395 at 455-464 and may interact with Sequence 732 in this patent.
ESEKNKTRTT

SEQ ID NO: 732 - protein, 10 aa, mycoplasma genitalium
  Sequence located in MG398 at 4-13 and may interact with Sequence 731 in this patent.
LRFLVLSPSG

SEQ ID NO: 733 - protein, 10 aa, mycoplasma genitalium
  Sequence located in MG397 at 303-312 and may interact with Sequence 734 in this patent.
SSKEKSINLV

SEQ ID NO: 734 - protein, 10 aa, mycoplasma genitalium
  Sequence located in MG468 at 1655-1664 and may interact with Sequence 733 in this patent.
NQVDAFLLGT

SEQ ID NO: 735 - protein, 10 aa, mycoplasma genitalium
  Sequence located in MG411 at 29-38 and may interact with Sequence 736 in this patent.
LFISFFVFLL

SEQ ID NO: 736 - protein, 10 aa, mycoplasma genitalium
  Sequence located in MG412 at 92-101 and may interact with Sequence 735 in this patent.
KEYAKENEKK

SEQ ID NO: 737 - protein, 10 aa, mycoplasma genitalium
  Sequence located in MG411 at 535-544 and may interact with Sequence 738 in this patent.
IFKIVLPSAL

SEQ ID NO: 738 - protein, 10 aa, mycoplasma genitalium
  Sequence located in MG421 at 16-25 and may interact with Sequence 737 in this patent.
KGARENNLKN

SEQ ID NO: 739 - protein, 10 aa, mycoplasma genitalium
  Sequence located in MG412 at 61-70 and may interact with Sequence 740 in this patent.
VEISVQAGGS

SEQ ID NO: 740 - protein, 10 aa, mycoplasma genitalium
  Sequence located in MG468 at 1113-1122 and may interact with Sequence 739 in this patent.
TSTSLNRNFN

SEQ ID NO: 741 - protein, 10 aa, mycoplasma genitalium
  Sequence located in MG421 at 39-48 and may interact with Sequence 742 in this patent.
TGLSGSGKSS

SEQ ID NO: 742 - protein, 10 aa, mycoplasma genitalium
  Sequence located in MG421 at 39-48 and may interact with Sequence 741 in this patent.
TGLSGSGKSS

SEQ ID NO: 743 - protein, 10 aa, mycoplasma genitalium
  Sequence located in MG421 at 492-501 and may interact with Sequence 744 in this patent.
RRASTLSGGE

SEQ ID NO: 744 - protein, 10 aa, mycoplasma genitalium
  Sequence located in MG425 at 48-57 and may interact with Sequence 743 in this patent.
PTGTGKTAAF

SEQ ID NO: 745 - protein, 10 aa, mycoplasma genitalium
  Sequence located in MG421 at 491-500 and may interact with Sequence 746 in this patent.
ARRASTLSGG

SEQ ID NO: 746 - protein, 10 aa, mycoplasma genitalium
  Sequence located in MG425 at 47-56 and may interact with Sequence 745 in this patent.
SPTGTGKTAA

SEQ ID NO: 747 - protein, 10 aa, mycoplasma genitalium
  Sequence located in MG422 at 673-682 and may interact with Sequence 748 in this patent.
LKKTNKKIKR

SEQ ID NO: 748 - protein, 10 aa, mycoplasma genitalium
  Sequence located in MG441 at 50-59 and may interact with Sequence 747 in this patent.
SLYLLVRLFQ

SEQ ID NO: 749 - protein, 10 aa, mycoplasma genitalium
  Sequence located in MG439 at 47-56 and may interact with Sequence 750 in this patent.
AVTFLKKGYS

SEQ ID NO: 750 - protein, 10 aa, mycoplasma genitalium
  Sequence located in MG439 at 47-56 and may interact with Sequence 749 in this patent.
AVTFLKKGYS

SEQ ID NO: 751 - protein, 10 aa, mycoplasma genitalium
  Sequence located in MG439 at 157-166 and may interact with Sequence 752 in this patent.
TGIVGSDNTS

SEQ ID NO: 752 - protein, 10 aa, mycoplasma genitalium
  Sequence located in MG439 at 157-166 and may interact with Sequence 751 in this patent.
TGIVGSDNTS

SEQ ID NO: 753 - protein, 10 aa, mycoplasma genitalium
  Sequence located in MG457 at 564-573 and may interact with Sequence 754 in this patent.
EITTGASSDF

SEQ ID NO: 754 - protein, 10 aa, mycoplasma genitalium
  Sequence located in MG457 at 564-573 and may interact with Sequence 753 in this patent.
EITTGASSDF

SEQ ID NO: 755 - protein, 10 aa, mycoplasma genitalium
  Sequence located in MG015 at 184-193 and may interact with Sequence 2 in this patent.
SFAFLKKSKT

SEQ ID NO: 756 - protein, 10 aa, mycoplasma genitalium
  Sequence located in MG015 at 184-193 and may interact with Sequence 1 in this patent.
SFAFLKKSKT

SEQ ID NO: 757 - protein, 10 aa, mycoplasma genitalium
  Sequence located in MG049 at 102-111 and may interact with Sequence 4 in this patent.
GDPLRAKWIA

SEQ ID NO: 758 - protein, 10 aa, mycoplasma genitalium
  Sequence located in MG049 at 102-111 and may interact with Sequence 3 in this patent.
GDPLRAKWIA

SEQ ID NO: 759 - protein, 10 aa, mycoplasma genitalium
  Sequence located in MG065 at 104-113 and may interact with Sequence 6 in this patent.
ETVIASYNSF

SEQ ID NO: 760 - protein, 10 aa, mycoplasma genitalium
  Sequence located in MG065 at 104-113 and may interact with Sequence 5 in this patent.
ETVIASYNSF

SEQ ID NO: 761 - protein, 10 aa, mycoplasma genitalium
  Sequence located in MG271 at 243-252 and may interact with Sequence 8 in this patent.
LFYTVNGVEQ

SEQ ID NO: 762 - protein, 10 aa, mycoplasma genitalium
  Sequence located in MG271 at 243-252 and may interact with Sequence 7 in this patent.
LFYTVNGVEQ

SEQ ID NO: 763 - protein, 10 aa, mycoplasma genitalium
  Sequence located in MG276 at 85-94 and may interact with Sequence 10 in this patent.
ANKLPGQLIS

SEQ ID NO: 764 - protein, 10 aa, mycoplasma genitalium
  Sequence located in MG276 at 85-94 and may interact with Sequence 9 in this patent.
ANKLPGQLIS

SEQ ID NO: 765 - protein, 10 aa, mycoplasma genitalium
  Sequence located in MG027 at 94-103 and may interact with Sequence 12 in this patent.
SGKKIDLLTT

SEQ ID NO: 766 - protein, 10 aa, mycoplasma genitalium
  Sequence located in MG027 at 94-103 and may interact with Sequence 11 in this patent.
SGKKIDLLTT

SEQ ID NO: 767 - protein, 10 aa, mycoplasma genitalium
  Sequence located in MG088 at 3-12 and may interact with Sequence 14 in this patent.
KNRALKRTVL

SEQ ID NO: 768 - protein, 10 aa, mycoplasma genitalium
  Sequence located in MG088 at 3-12 and may interact with Sequence 13 in this patent.
KNRALKRTVL

SEQ ID NO: 769 - protein, 10 aa, mycoplasma genitalium
  Sequence located in MG088 at 4-13 and may interact with Sequence 16 in this patent.
NRALKRTVLP

SEQ ID NO: 770 - protein, 10 aa, mycoplasma genitalium
  Sequence located in MG088 at 2-11 and may interact with Sequence 15 in this patent.
RKNRALKRTV

SEQ ID NO: 771 - protein, 10 aa, mycoplasma genitalium
  Sequence located in MG130 at 385-394 and may interact with Sequence 18 in this patent.
ARIGARSDSS

SEQ ID NO: 772 - protein, 10 aa, mycoplasma genitalium
  Sequence located in MG130 at 385-394 and may interact with Sequence 17 in this patent.
ARIGARSDSS

SEQ ID NO: 773 - protein, 10 aa, mycoplasma genitalium
  Sequence located in MG149 at 180-189 and may interact with Sequence 20 in this patent.
IFLVIDYKKD

SEQ ID NO: 774 - protein, 10 aa, mycoplasma genitalium
  Sequence located in MG149 at 180-189 and may interact with Sequence 19 in this patent.
IFLVIDYKKD

SEQ ID NO: 775 - protein, 10 aa, mycoplasma genitalium
  Sequence located in MG149 at 181-190 and may interact with Sequence 22 in this patent.
FLVIDYKKDS

SEQ ID NO: 776 - protein, 10 aa, mycoplasma genitalium
  Sequence located in MG149 at 179-188 and may interact with Sequence 21 in this patent.
GIFLVIDYKK

SEQ ID NO: 777 - protein, 10 aa, mycoplasma genitalium
  Sequence located in MG169 at 40-49 and may interact with Sequence 24 in this patent.
KARKSGLTRL

SEQ ID NO: 778 - protein, 10 aa, mycoplasma genitalium
  Sequence located in MG169 at 40-49 and may interact with Sequence 23 in this patent.
KARKSGLTRL

SEQ ID NO: 779 - protein, 10 aa, mycoplasma genitalium
  Sequence located in MG194 at 106-115 and may interact with Sequence 26 in this patent.
LYQVIDNLVE

SEQ ID NO: 780 - protein, 10 aa, mycoplasma genitalium
  Sequence located in MG194 at 106-115 and may interact with Sequence 25 in this patent.
LYQVIDNLVE

SEQ ID NO: 781 - protein, 10 aa, mycoplasma genitalium
  Sequence located in MG194 at 181-190 and may interact with Sequence 28 in this patent.
TNNPDIRVVS

SEQ ID NO: 782 - protein, 10 aa, mycoplasma genitalium
  Sequence located in MG194 at 181-190 and may interact with Sequence 27 in this patent.
TNNPDIRVVS

SEQ ID NO: 783 - protein, 10 aa, mycoplasma genitalium
  Sequence located in MG194 at 182-191 and may interact with Sequence 30 in this patent.
NNPDIRVVSL

SEQ ID NO: 784 - protein, 10 aa, mycoplasma genitalium
  Sequence located in MG194 at 180-189 and may interact with Sequence 29 in this patent.
KTNNPDIRVV

SEQ ID NO: 785 - protein, 10 aa, mycoplasma genitalium
  Sequence located in MG226 at 95-104 and may interact with Sequence 32 in this patent.
KANNNIIIGL

SEQ ID NO: 786 - protein, 10 aa, mycoplasma genitalium
  Sequence located in MG226 at 95-104 and may interact with Sequence 31 in this patent.
KANNNIIIGL

SEQ ID NO: 787 - protein, 10 aa, mycoplasma genitalium
  Sequence located in MG321 at 591-600 and may interact with Sequence 34 in this patent.
RTASTSGSSS

SEQ ID NO: 788 - protein, 10 aa, mycoplasma genitalium
  Sequence located in MG321 at 591-600 and may interact with Sequence 33 in this patent.
RTASTSGSSS

SEQ ID NO: 789 - protein, 10 aa, mycoplasma genitalium
  Sequence located in MG322 at 274-283 and may interact with Sequence 36 in this patent.
LFDGFEAIKK

SEQ ID NO: 790 - protein, 10 aa, mycoplasma genitalium
  Sequence located in MG322 at 274-283 and may interact with Sequence 35 in this patent.
LFDGFEAIKK

SEQ ID NO: 791 - protein, 10 aa, mycoplasma genitalium
  Sequence located in MG324 at 23-32 and may interact with Sequence 38 in this patent.
AILIGSDQNR

SEQ ID NO: 792 - protein, 10 aa, mycoplasma genitalium
  Sequence located in MG324 at 23-32 and may interact with Sequence 37 in this patent.
AILIGSDQNR

SEQ ID NO: 793 - protein, 10 aa, mycoplasma genitalium
  Sequence located in MG376 at 17-26 and may interact with Sequence 40 in this patent.
SHKKTGFLVT

SEQ ID NO: 794 - protein, 10 aa, mycoplasma genitalium
  Sequence located in MG376 at 17-26 and may interact with Sequence 39 in this patent.
SHKKTGFLVT

SEQ ID NO: 795 - protein, 10 aa, mycoplasma genitalium
  Sequence located in MG391 at 392-401 and may interact with Sequence 42 in this patent.
SARGAGSSRA

SEQ ID NO: 796 - protein, 10 aa, mycoplasma genitalium
  Sequence located in MG391 at 392-401 and may interact with Sequence 41 in this patent.
SARGAGSSRA

SEQ ID NO: 797 - protein, 10 aa, mycoplasma genitalium
  Sequence located in MG421 at 39-48 and may interact with Sequence 44 in this patent.
TGLSGSGKSS

SEQ ID NO: 798 - protein, 10 aa, mycoplasma genitalium
  Sequence located in MG421 at 39-48 and may interact with Sequence 43 in this patent.
TGLSGSGKSS

SEQ ID NO: 799 - protein, 10 aa, mycoplasma genitalium
  Sequence located in MG439 at 47-56 and may interact with Sequence 46 in this patent.
AVTFLKKGYS

SEQ ID NO: 800 - protein, 10 aa, mycoplasma genitalium
  Sequence located in MG439 at 47-56 and may interact with Sequence 45 in this patent.
AVTFLKKGYS

SEQ ID NO: 801 - protein, 10 aa, mycoplasma genitalium
  Sequence located in MG439 at 157-166 and may interact with Sequence 48 in this patent.
TGIVGSDNTS

SEQ ID NO: 802 - protein, 10 aa, mycoplasma genitalium
  Sequence located in MG439 at 157-166 and may interact with Sequence 47 in this patent.
TGIVGSDNTS

SEQ ID NO: 803 - protein, 10 aa, mycoplasma genitalium
  Sequence located in MG457 at 564-573 and may interact with Sequence 50 in this patent.
EITTGASSDF

SEQ ID NO: 804 - protein, 10 aa, mycoplasma genitalium
  Sequence located in MG457 at 564-573 and may interact with Sequence 49 in this patent.
EITTGASSDF

(19) United States
(12) Patent Application Publication
(10) Pub. No.: US 2003/0199011 A1
(43) Pub. Date: Oct. 23, 2003

(54) Complementary peptide ligands generated from microbial genome sequences

(76) Inventors: Garth W. Roberts, Cambridge (GB); Jonathan R. Heal, Hilbury (GB)

Correspondence Address: FINNEGAN, HENDERSON, FARABOW, GARRETT & DUNNER, LLP, 1300 I STREET, NW, WASHINGTON, DC, 20005, US

(21) Appl. No.: 09/573,822

(22) Filed: May 18, 2000

(30) Foreign Application Priority Data
Dec. 13, 1999 (GB) 9929466.2

(51) Int. Cl. G01N 33/554; G01N 33/569; G06F 19/00; G01N 33/48; G01N 33/50; C07K 14/195

(52) U.S. Cl. 435/7.32; 702/20; 530/350

(57) ABSTRACT

In the current invention the application of our novel informatics approach to the databases containing nucleotide and peptide sequences from pathogens generates the sequence of many peptides which form the basis of an innovative and novel approach to developing anti-infective drugs.

This invention claims the use of specific complementary peptide to the proteins encoded in the genomes of known pathogens as reagents and drugs for drug discovery programmes.

At present more than 20 microbial genomes have been sequenced and over 80 are on going sequencing projects. Many of the microbes that have been selected are human pathogens and are responsible for a large proportion of the global burden of infectious disease.

### BACKGROUND

Specific protein interactions are critical events in most biological processes and a clear idea of the way proteins interact, their three dimensional structure and the types of molecules which might block or enhance interaction are critical aspects of the science of drug discovery in the pharmaceutical industry.

Proteins are made up of strings of amino acids and each amino acid in a string is coded for by a triplet of nucleotides present in DNA sequences (Stryer 1997). The linear sequence of DNA code is read and translated by a cell's synthetic machinery to produce a linear sequence of amino acids that then fold to form a complex three-dimensional protein.

In general it is held that the primary structure of a protein determines its tertiary structure. A large volume of work supports this view and many sources of software are available to the scientists in order to produce models of protein structures (Sansom 1998). In addition, a considerable effort is underway in order to build on this principle and generate a definitive database demonstrating the relationships between primary and tertiary protein structures. This endeavour is likened to the human genome project and is estimated to have a similar cost (Gaasterland 1998).

The binding of large proteinaceous signalling molecules (such as hormones) to cellular receptors regulates a substantial portion of the control of cellular processes and functions. These protein-protein interactions are distinct from the interaction of substrates to enzymes or small molecule ligands to seven-transmembrane receptors. Protein-protein interactions occur over relatively large surface areas, as opposed to the interactions of small molecule ligands with serpentine receptors, or enzymes with their substrates, which usually occur in focused “pockets” or “clefts”. Thus, protein-protein targets are non-traditional and the pharmaceutical community has had very limited success in developing drugs that bind to them using currently available approaches to lead discovery. High throughput screening technologies in which large (combinatorial) libraries of synthetic compounds are screened against a target protein(s) have failed to produce a significant number of lead compounds.

Many major diseases result from the inactivity or hyperactivity of large protein signalling molecules. For example, diabetes mellitus results from the absence or ineffectiveness of insulin, and dwarfism from the lack of growth hormone. Thus, simple replacement therapy with recombinant forms of insulin or growth hormone heralded the beginnings of the biotechnology industry. However, nearly all drugs that target protein-protein interactions or that mimic large protein signalling molecules are also large proteins. Protein drugs are expensive to manufacture, difficult to formulate, and must be given by injection or topical administration.

It is generally believed that because the binding interfaces between proteins are very large, traditional approaches to drug screening or design have not been successful. In fact, for most protein-protein interactions, only small subsets of the overall intermolecular surfaces are important in defining binding affinity. “One strongly suspects that the many crevices, canyons, depressions and gaps, that punctuate any protein surface are places that interact with numerous micro- and macro-molecular ligands inside the cell or in the extra-cellular spaces, the identity of which is not known” (Goldstein 1998).

Despite these complexities, recent evidence suggests that protein-protein interfaces are tractable targets for drug design when coupled with suitable functional analysis and more robust molecular diversity methods. For example, the interface between hGH and its receptor buries ˜1300 Sq. Angstroms of surface area and involves 30 contact side chains across the interface. However, alanine-scanning mutagenesis shows that only eight side-chains at the centre of the interface (covering an area of about 350 Sq. Angstroms) are crucial for affinity. Such “hot spots” have been found in numerous other protein-protein complexes by alanine-scanning, and their existence is likely to be a general phenomenon.

The problem therefore is to define the small subset of regions that define the binding or functionality of the protein.

The important commercial reason for this is that a more efficient way of doing this would greatly accelerate the process of drug development.

These complexities are not insoluble problems and newer theoretical methods should not be ignored in the drug design process. Nonetheless, in the near future there are no good algorithms that allow one to predict protein binding affinities quickly, reliably, and with high precision (Sunesis website www.sunesis.com 17/9/99).

A process for the analysis of whole genome databases has been developed. Significant utility can be achieved within the pharmaceutical industry by searching and analysing protein and nucleotide sequence databases to identify complementary peptides which interact with their relevant target proteins.

These novel peptides can be used as lead ligands to facilitate drug design and development. This invention describes the application of this process to the databases containing nucleotide and protein sequence data from known pathogens (microbes, viruses, fungi and protozoa).

The process has been described in patent application number GB 9927485.4, filed 19th November 1999 for use in analysing, and manipulating the sequence data (both DNA and protein) found in large databases and its utility in conducting systematic searches to identify the sequences which code for the key intermolecular surfaces or “hot spots” on specific protein targets.

### THE INVENTION

In the current invention the application of our novel informatics approach to the databases containing nucleotide and peptide sequences from pathogens generates the sequence of many peptides which form the basis of an innovative and novel approach to developing anti-infective drugs.

This invention claims the use of specific complementary peptide to the proteins encoded in the genomes of known pathogens as reagents and drugs for drug discovery programmes.

The Need For New Approaches to Anti-Infective Drugs

For many bacterial and viral infectious diseases, the remarkable genetic variability and adaptability of microorganisms constitutes a major problem for clinicians and patients (see EXAMPLE 1). In developed countries people living in poor socioeconomic conditions and expanding elderly populations are increasingly susceptible to relatively innocuous infectious agents. Resistance is an ongoing problem in intensive care units where high levels of antibiotics are used to combat infections. Consequently, there is a pressing need to understand how bacteria acquire resistance to antibiotics and to develop new agents to combat them.

The need for new antibiotics is urgent as nearly 9 million people in the United States are affected by drug-resistant bacterial infections each year and are the cause of death for approximately 60,000 of these individuals.

The development of antibiotics was a major advance in combating bacterial infections. However, antiviral agents have not been nearly as effective. Since viruses are totally dependent on their host cells and carry little that is unique to them, it has proved difficult to obtain inhibitory agents which will not adversely affect the normal functions of the cell. Selected stages of the replication cycle are potentially vulnerable to inhibition by suitable agents and a few are in clinical use.

Applications of genomic research and systematic DNA sequence analysis will open new avenues for research in immunology, therapeutics and drug development, including vaccines and new antibiotics.

For instance, two peptide therapies have been developed for the treatment of HIV. One, T-20, inhibits the fusion of HIV with the host cell. Fusion of the viral envelope with a target cell membrane is required for the initiation of infection and therefore, virus replication.

A synthetic version of the naturally occurring peptide thymosin alpha 1 has been developed to treat Hepatitis B and C infections. The peptide, Zadaxin, works by boosting the body's immune's ability to produce T cells that are the body's most potent defence against infectious diseases. It promotes the maturation of disease fighting T cells, which are involved in the control of various immune responses.

### APPLICATION OF THE DATA MINING PROCESS TO THE ANALYSIS OF PATHOGEN GENOMES

We have applied our computational approach with its novel algorithms for generating complementary peptides to the known pathogen nucleotide and peptide sequence databases.

There are over 20 completed pathogen genomes in public databases (GOLD, Genomes On Line Database, http://geta.life.uiuc.edu/˜nikos/genomes.html, 25/10/99). Of these, there are 16 eubacterium, 6 archeabacterium and 1 unicellular eukaryote (in addition the genome of the nematode worm C. elegans is complete). At least another 84 prokaryote and 27 eukaryote genomes are partially sequenced and many are nearing completion, including the human genome. High-throughput genome sequencing is now making it possible to compare organisms at the level of whole genomes. This will allow important clinically relevant differences between man and viral/bacterial and fungal pathogens to be made.

Whole genome sequences represent a huge resource of data for the discovery and utilisation of biologically important complementary peptides. The catalogues detailed in this patent cover all available pathogen genomes. A series of Tables (Tables 1 to 5) detailing the various pathogens and their genomic databases which have been processed using our method are shown below.

Sequence data from completed genomes is downloaded from the NCBI (National Centre for Biotechnology Information), http://www.ncbi.nlm.nih.gov/Entrez/Genome/org.html, and analysed for complementary peptide sequences both intra-molecular (within a protein) and inter-molecular (between proteins) as described in patent application number GB 9927485.4 filed 19th November 1999.

A set of inter-molecular complementary peptide sequences, frame size 10, was generated for each gene within a pathogen genome (see EXAMPLE 2).

Sets of shorter ‘daughter’ sequences of frame size 5,6,7,8 or 9 can also be derived from these sequences (EXAMPLE 3).

A catalogue of complementary intra molecular peptides frame size 10 (average 3 per gene) was generated for each gene within a pathogen genome (see EXAMPLE 4).

Sets of shorter ‘daughter’ sequences of frame size 5,6,7,8 or 9 can also be derived from these sequences (EXAMPLE 5).

Each complementary peptide sequence has a unique identifying number in the catalog and peptides are categorised as either inter-molecular or intra-molecular peptides within each genome as shown in the table below (and in EXAMPLES 2,4 and in the genomes noted in EXAMPLES 6 and 7):

| | Inter-molecular | Intra-molecular |
|---|---|---|
| Genome | peptides | peptides |
| Borrelia burgdorferi | | |
| Chlamydia Pneumoniae | | |
| Chlamydia Trachomatis | | |
| Echerichia Coli | | |
| Haemophilus Influenzae | | |
| Helicobacter Pylori | | |
| Mycobacterium tuberculosis | | |
| Mycoplasma genitalium | 1-754 | 755-804 |
| Mycoplasma pneumoniae | | |
| Rickettsia Prowazekii | | |
| Treponema Pallidum | | |

Utilizing our novel approach we were able to establish the sequences of complementary peptides that have the potential to interact with and alter the functionality of the relevant protein coded for by its gene. Furthermore the second analysis provides information as to the regions on other proteins which might interact with the first protein (its ‘molecular partners’ in physiological functions).

The peptide sequences described herein can be readily made into peptides by a multitude of methods. The peptides made from the sequences described in this patent will have considerable utility as tools for functional genomics studies, reagents for the configuration of high-throughput screens, a starting point for medicinal chemistry manipulation, peptide mimetics, and therapeutic agents in their own right.

The generation of complementary peptides to nucleotide and protein sequences from pathogen genomes offers a substantial opportunity for delivering novel and innovative leads to drug development programmes in the area of anti-infective medicine.

The process of patent application number GB9927485.4 will now be described below. The examples of this present application are the result of applying that process to a selected microbial database :- it will readily be appreciated that use of the process on other microbial databases will yield peptide sequences and catalogues of intra- and inter-molecular complementary peptides specific to the other microbial databases (e.g. the microbial databases tabulated above, and in Tables 1 to 5).

The current problems associated with design of complementary peptides are:

A lack of understanding of the forces of recognition between complementary peptides

An absence of software tools to facilitate searching and selecting complementary peptide pairs from within a protein database

A lack of understanding of statistical relevance/distribution of naturally encoded complementary peptides and how this corresponds to functional relevance.

Based on these shortfalls, our process provides the following technological advances in this field:

A mini library approach to define forces of recognition between human Interleukin (IL) 1βand its complementary peptides.

A high throughput computer system to analyse an entire database for intra/inter-molecular complementary regions.

Studies into preferred complementary peptide pairings between IL-1βand its complementary ligand reveal the importance of both the genetic code and complementary hydropathy for recognition. Specifically, for our example, the genetic code for a region of protein codes for the complementary peptide with the highest affinity. An important observation is that this complementary peptide maps spatially and by residue hydropathic character to the interacting portion of the IL-1R receptor, as elucidated by the X-ray crystal structure Brookhaven reference pdb2itb.ent.

Using these novel observations as guiding principles for analysis, we have developed a computational analysis system to evaluate the statistical and functional relevance of intra/inter- molecular complementary sequences.

This process provides significant benefits for those interested in:

The analysis and acquisition of peptide sequences to be used in the understanding of protein-protein interactions.

The development of peptides or small molecules which could be used to manipulate these interactions.

The advantages of this process to previous work in this field include:

Using a valid statistical model. Previously, complementary mappings within protein structures has been statistically validated by assuming that the occurrence of individual amino acids is equally weighted at 1/20 (Baranyi, 1995). Our statistical model takes into account the natural occurrence of amino acids and thus generates probabilities dependent on sequence rather than content per se.

Facilitation of batch searching of an entire database. Previously, investigations into the, significance of naturally encoded complementary related sequences have been limited to small sample sizes with non-automated methods. The invention allows for analysis of an entire database at a time, overcoming the sampling problem, and providing for the first time an overview or ‘map’ of complementary peptide sequences within known protein sequences.

The ability to map complementary sequences as a function of frame size and percentage antisense amino acid content. Previously, no consideration has been given to the significance of the frame length of complementary sequences. Our process produces a statistical map as a function of frame size and percentage complementary residue content such that the statistical importance of how nature selects these frames may be evaluated.

### BRIEF DESCRIPTION OF DRAWINGS

The process is described with reference to accompanying drawings. In the drawings, like reference numbers indicate identical or functionally similar elements.

FIG (1) shows a block diagram illustrating one embodiment of a method of the present invention

FIG (2) shows a block diagram illustrating one embodiment for carrying out Step 4 in FIG (1)

FIG (3) shows a block diagram illustrating one embodiment for carrying out Step 5 in FIG (1)

FIG (4) shows a block diagram illustrating one embodiment for carrying out Step 8 in FIG (2) and (3)

FIG (5) shows a block diagram illustrating one embodiment for carrying out Step 8 in FIG (2) and (3)

FIG (6) shows a block diagram illustrating one embodiment for carrying out Step 6 in FIG (1)

### A description of the analytical process

The software, ALS (antisense ligand searcher), performs the following tasks:

Given the input of two amino acid sequences, calculates the position, number and probability of the existence of intra- (within a protein) and inter- (between proteins) molecular antisense regions. ‘Antisense’ refers to relationships between amino acids specified in EXAMPLES 8 and 9 (both 5′−>3′ derived and 3′>−5′ derived coding schemes).

Allows sequences to be inputted manually through a suitable user interface (UI) and also through a connection to a database such that automated, or batch, processing can be facilitated.

Provides a suitable database to store results and an appropriate interface to allow manipulation of this data.

Allows generation of random sequences to function as experimental controls.

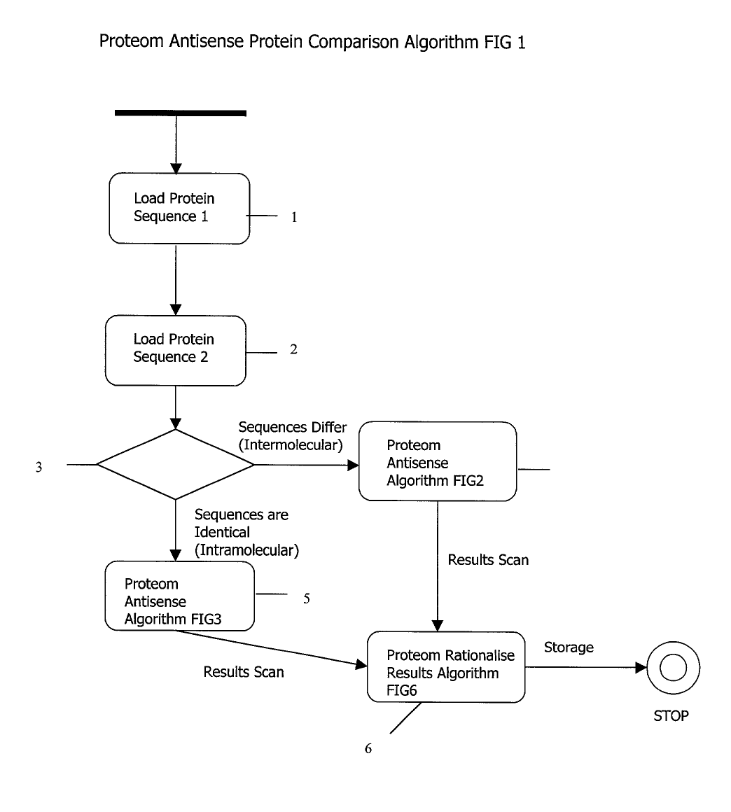

Diagrams describing the algorithms involved in this software are shown in FIGS. 1-5.

### DETAILED DESCRIPTION

1. Overview

The present process is directed toward a computer-based process, a computer-based system and/or a computer program product for analysing antisense relationships between protein or DNA sequences. The method of the embodiment provides a tool for the analysis of protein or DNA sequences for antisense relationships. This embodiment covers analysis of DNA or protein sequences for intramolecular (within the same sequence) antisense relationships or inter-molecular (between 2 different sequences) antisense relationships. This principle applies whether the sequence contains amino acid information (protein) or DNA information, since the former may be derived from the latter.

The overall process is to facilitate the batch analysis of an entire genome (collection of genes/and or protein sequences) for every possible antisense relationship of both inter- and intra-molecular nature. For the purpose of example it will be described here how a protein sequence database may be analysed by the methods described.

The program runs in two modes. The first mode (Intermolecular) is to select the first protein sequence in the databases and then analyse the antisense relationships between this sequence and all other protein sequences, one at a time. The program then selects the second sequence and repeats this process. This continues until all of the possible relationships have been analysed. The second mode (Intramolecular) is where each protein sequence is analysed for antisense relationships within the same protein and thus each sequence is loaded from the database and analysed in turn for these properties. Both operational modes use the same core algorithms for their processes. The core algorithms are described in detail below.

An example of the output from this process is a list of proteins in the database that contain highly improbable numbers of intramolecular antisense frames of size 10 (frame size is a section of the main sequence, it is described in more detail below).

2. Method

For the purpose of example protein sequence 1 is ATRGRDSRDERSDERTD and protein sequence 2 is GTFRTSREDSTYSGDTDFDE (universal 1 letter amino acid codes used).

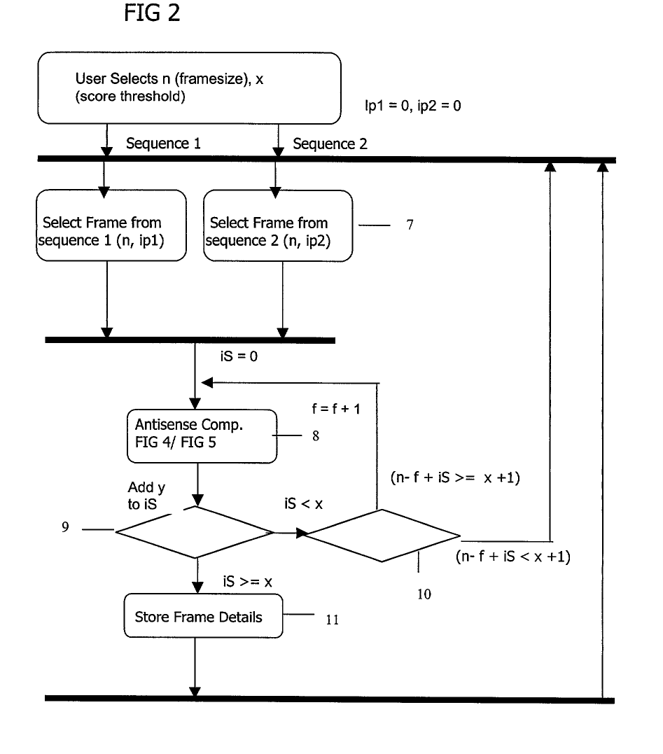

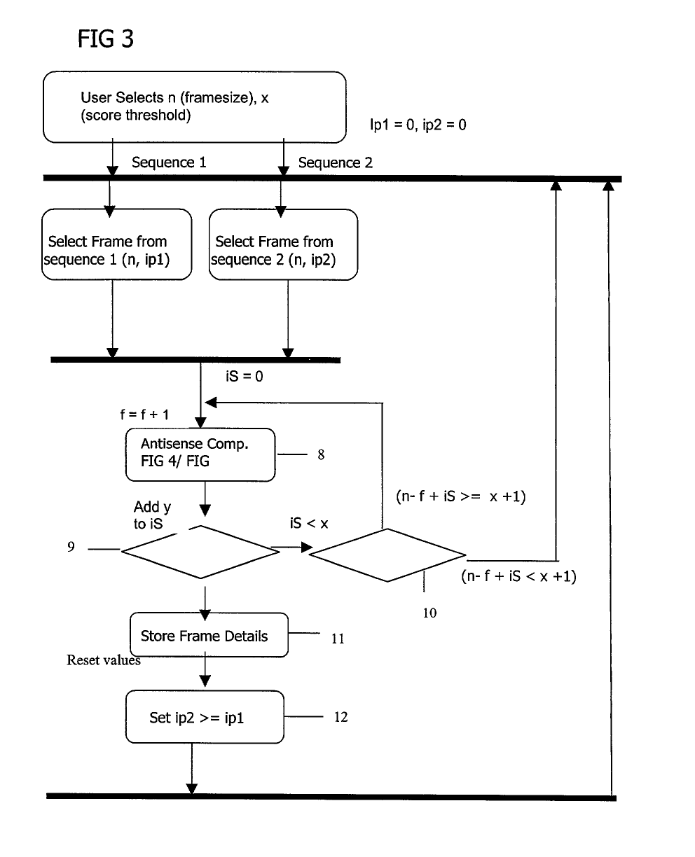

In step 1 (see FIG. 1), a protein sequence, sequence 1, is loaded. The protein sequence consists of an array of universally recognised amino acid one letter codes, e.g. ‘ADTRGSRD’. The source of this sequence can be a database, or any other file type. Step 2, is the same operation as for step 1, except sequence 2 is loaded. Decision step 3 involves comparing the two sequences and determining whether they are identical, or whether they differ. If they differ, processing continues to step 4, described in FIG. 2, otherwise processing continues to step 5, described in FIG. 3.

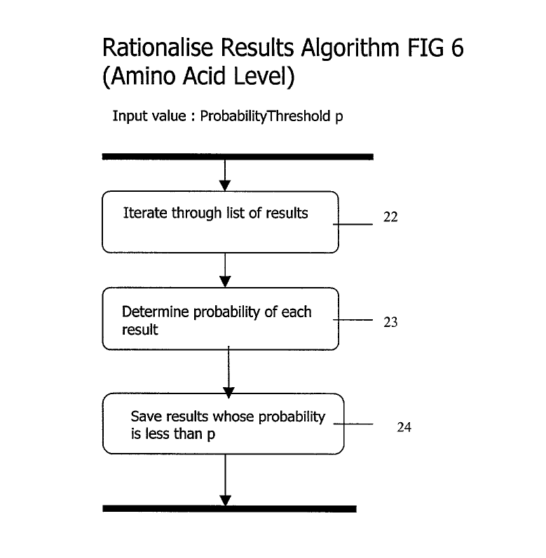

Step 6 analyses the data resulting from either step 4, or step 5, and involves an algorithm described in FIG. 6.

Description of parameters used in FIG. 2

| Name | Description |
|---|---|
| N | Framesize - the number of amino acids that make up each ‘frame’ |
| X | Score threshold - the number of amino acids that have to fulfil the |
| | antisense criteria within a given frame for that frame |
| | to be stored for analysis |
| Y | Score of individual antisense comparison (either 1 or 0) |
| IS | Running score for frame - (sum of y for frame) |
| ip1 | Position marker for Sequence 1 - used to track location of |
| | selected frame for sequence 1 |
| ip2 | Position marker for Sequence 2 - used to track location of |
| | selected frame for sequence 1 |
| F | Current position in frame |

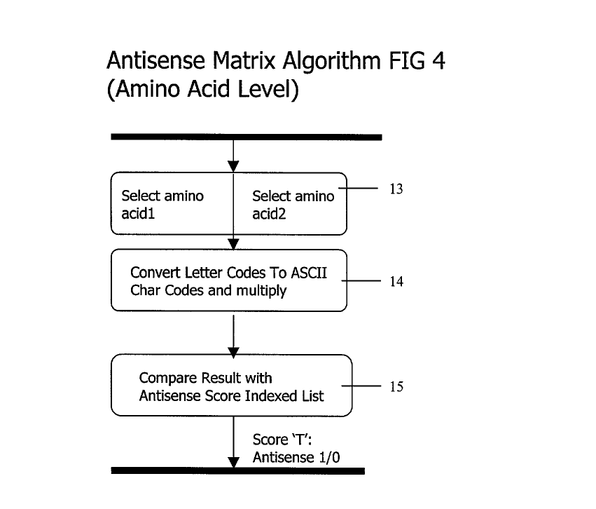

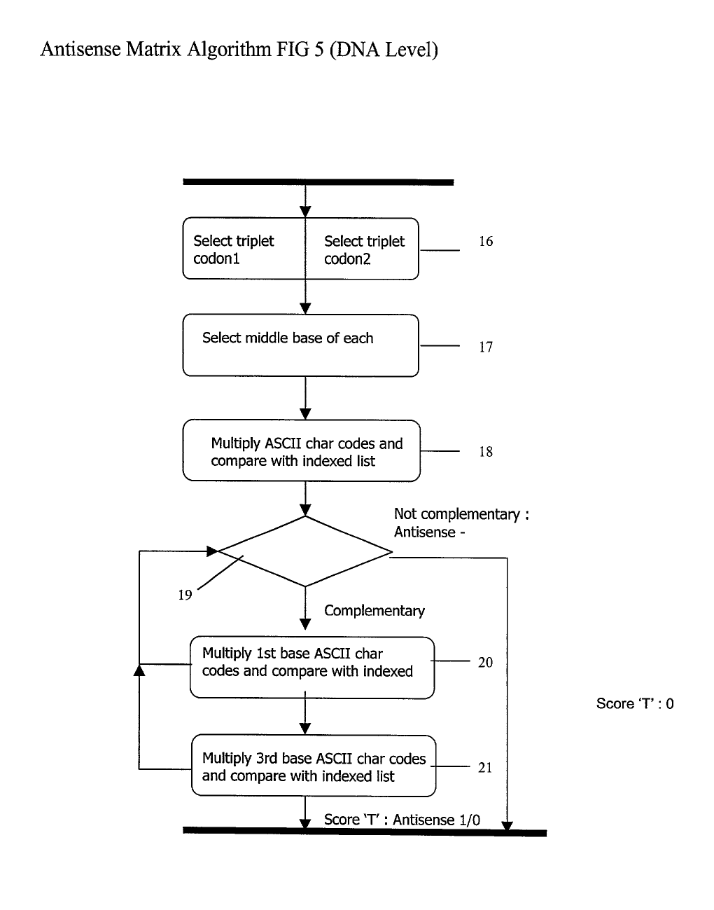

In Step 7, a ‘frame’ is selected for each of the proteins selected in steps 1 and 2. A ‘frame’ is a specific section of a protein sequence. For example, for sequence 1, the first frame of length ‘5’ would correspond to the characters ‘ATRGR’. The user of the program decides the frame length as. an input value. This value corresponds to parameter ‘n’ in FIG. 2. A frame is selected from each of the protein sequences (sequence 1 and sequence 2). Each pair of frames that are selected are aligned and frame position parameter f is set to zero. The first pair of amino acids are ‘compared’ using the algorithm shown in FIG. 4/FIG 5. The score output from this algorithm (y, either one or zero) is added to a aggregate score for the frame iS. In decision step 9 it is determined whether the aggregate score iS is greater than the Score threshold value (x). If it is then the frame is stored for further analyisis. If it is not then decision step 10 is implemented. In decision step 10, it is determined whether it is possible for the frame to yield the score threshold (x). If it can, the frame processing continues and f is incremented such that the next pair of amino acids are compared. If it cannot, the loop exits and the next frame is selected. The position that the frame is selected from the protein sequences is determined by the parameter ip1 for sequence 1 and ip2 for sequence 2 (refer to FIG. 2). Each time steps 7 to 10 or 7 to 11 are completed, the value of ip1 is zeroed and then incremented until all frames of sequence 1 have been analysed against the chosen frame of sequence 2. When this is done, ip2 is then incremented and the value of ip1 is incremented until all frames of sequence 1 have been analysed against the chosen frame of sequence 2. This process repeats and terminates when ip2 is equal to the length of sequence 2. Once this process is complete, sequence 1 is reversed programmatically and the same analysis as described above is repeated. The overall effect of repeating steps 7 to 11 using each possible frame from both sequences is to facilitate step 8, the antisense scoring matrix for each possible combination of linear sequences at a given frame length.

FIG. 3 shows a block diagram of the algorithmic process that is carried out in the conditions described in FIG. 1. Step 12 is the only difference between the algorithms FIG. 2 and FIG. 3. In step 12, the value of ip2 (the position of the frame in sequence 2) is set to at least the value of ip1 at all times since as sequence I and sequence 2 are identical, if ip2 is less than ip1 then the same sequences are being searched twice.

FIGS. 4 and 5 describe the process in which a pair of amino acids (FIG. 4) or a pair of triplet codons are assessed for an antisense relationship. The antisense relationships are listed in EXAMPLES 8 and 9. In step 13, the currently selected amino acid from the current frame of sequence 1 and the currently selected amino acid from the current frame of sequence 2 (determined by parameter ‘f’ in FIG. 2/3) are selected. For example, the first amino acid from the first frame of sequence 1 would be ‘A’ and the first amino acid from the first frame of sequence 2 would be ‘G’. In step 14, the ASCII character codes for the selected single uppercase characters are determined and multiplied and, in step 15, the product compared with a list of precalculated scores, which represent the antisense relationships in EXAMPLES 8 and 9. If the amino acids are deemed to fulfil the criteria for an antisense relationship (the product matches a value in the precalculated list) then an output parameter ‘T’ is set to 1, otherwise the output parameter is set to zero.

Steps 16-21 relate to the case where the input sequences are DNA/RNA code rather the protein sequence. For example sequence 1 could be AAATTTAGCATG and sequence 2 could be TTTAAAGCATGC. The domain of the current invention includes both of these types of information as input values, since the protein sequence can be decoded from the DNA sequence, in accordance with the genetic code. Steps 16-21 determine antisense relationships for a given triplet codon. In step 16, the currently selected triplet codon for both sequences is ‘read’. For example, for sequence 1 the first triplet codon of the first frame would be ‘AAA’, and for sequence 2 this would be ‘TTT’. In step 17, the second character of each of these strings is selected. In step 18, the ASCII codes are multiplied and compared, in decision step 19, to a list to find out if the bases selected are ‘complementary’, in accordance with the rules of the genetic code. If they are, the first bases are compared in step 20, and subsequently the third bases are compared in step 21. Step 18 then determines whether the bases are ‘complementary’ or not. If the comparison yields a ‘non-complementary’ value at any step the routine terminates and the output score ‘T’ is set to zero. Otherwise the triplet codons are complementary and the output score T=1.

FIG. 6 illustrates the process of rationalising the results after the comparison of 2 protein or 2 DNA sequences. In step 22, the first ‘result’ is selected. A result consists of information on a pair of frames that were deemed ‘antisense’ in FIG's 2 or 3. This information includes location, length, score (i.e the sum of scores for a frame) and frame type (forward or reverse, depending on orientation of sequences with respect to one another). In step 23, the frame size, the score values and the length of the parent sequence are then used to calculate the probability of that frame existing. The statistics, which govern the probability of any frame existing, are described in the next section and refer to equations 1-4. If the probability is less than a user chosen value ‘p’, then the frame details are ‘stored’ for inclusion in the final result set (step 24).

### Statistical Basis of Program Operation

The number of complementary frames in a protein sequence can be predicted from appropriate use of statistical theory.

The probability of any one residue fitting the criteria for a complementary relationship with any other is defined by the groupings illustrated in EXAMPLE 8. Thus, depending on the residue in question, there are varying probabilities for the selection of a complementary amino acid. This is a result of an uneven distribution of possible partners. For example possible complementary partners for a tryptophan residue include only proline whilst glycine, serine, cysteine and arginine all fulfil the criteria as complementary partners for threonine. The probabilities for these residues aligning with a complementary match are thus 0.05 and 0.2 respectively. The first problem in fitting an accurate equation to describe the expected number of complementary frames within any sequence is integrating these uneven probabilities into the model. One solution is to use an average value of the relative abundance of the different amino acids in natural sequences. This is calculated by equation 1

v=ΣR*N 1

Where v =probability sum, R =fractional abundance of amino acid in e.coli proteins, N=number of complementary partners specified by genetic code.

This value (p) is calculated as 2.98. The average probability (p) of selecting a complementary amino acid is thus 2.98/20 =0.149.

For a single ‘frame’ of size (n) the probability (C) of pairing a number of complementary amino acids (r) can be described by the binomial distribution (equation 2)

$$C = \frac{n !}{( n - r ) ! r !} {{p}^{r} ( 1 - p )}^{n - r} \qquad 2$$

With this information we can predict that the expected number (Ex) of complementary frames in a protein to be:

$$Ex = 2 {( S - n )}^{2} \frac{n !}{( n - r ) ! r !} {{p}^{r} ( 1 - p )}^{n - r} \qquad 3$$

Where S =protein length, n =frame size, r =number of complementary residues required for a frame and p =0.149. If r =n, representing that all amino acids in a frame have to fulfil a complementary relationship, the above equation simplifies to:

Ex =2(S - n)²pⁿ 4

For a population of randomly assembled amino acid chains of a predetermined length we would expect the number of frames fulfilling the complementary c riteria in the search algorithm to vary in accordance with a normal distribution.

Importantly, it is possible to standardise results such that given a calculated mean (μ) and standard deviation (σ) for a population it is possible to determine the probability of any specific result occurring. Standardisation of the distribution model is facilitated by the following relation: -

$$Z = \frac{X - μ}{σ} \qquad 5$$

Where Xis an single value (result) in a population. If we are considering complementary frames with a single protein structure then the above statistical model requires further analysis. In particular, the possibility exists that a region may be complementary to itself, as indicated in the diagram below.

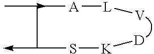

Reverse turn motifs within proteins. A region of protein may be complementary to itself. In this scenario, A-S, L-K and V-D are complementary partners. A six amino acid wide frame would thus be reported (in reverse orientation). A frame of this type is only specified by half of the residues in the frame. Such a frame is called a reverse turn.

In this scenario, once half of the frame length has been selected with complementary partners, there is a finite probability that those partners are the sequential neighbouring amino acids to those already selected. The probability of this occurring in any protein of any sequence is:

Ex =pf¹²(S-f) 7

Where f is the frame size for analysis, and S is the sequence length and p is the average probability of choosing an antisense amino acid.

The software of the embodiment incorporates all of the statistical models reported above such that it may assess whether a frame qualifies as a forward frame, reverse frame, or is reverse turn.

TABLE ONE
Published microbial genomes (taken from http://www.tigr.org/tdb/mdb/mdb.html,
25/10/99), all completed sequences can be accessed via
http://www.ncbi.nlm.nih.gov/Entrez/Genome/org.html
Key: A; Archaea, B; Eubacteria, E; Eukaryote

| enome | Strain | Key | Disease | Web link | Publication |
|---|---|---|---|---|---|
| Aeropyrum pernix | K1 | A | aerobic hyper-thermophilic | http://www.mild.nite.go.jp/APE | Kawarabayasi et |
| Genome Size (Mb) 1.67 | | | crenarchaeon | K1/index.html | al., DNA |
| | | | | | Research 6: 83- |
| | | | | | 101 (1999) |
| Aquifex aeolicus | VF5 | B | Hyperthermophilic | http://www.ncgr.org/microbe/aq | Deckert et al., |
| Genome Size (Mb) 1.50 | | | chemolithoautotroph | uifextxt.html | Nature 392:353 |
| | | | | | (1998) |
| Archaeoglobus fulgidus | DSM4304 | A | Hyperthermophilic marine | http://www.tigr.org/tdb/CMR/ga | Klenk et |
| Genome Size (Mb) 2.18 | | | Sulphate reducing | f/htmls/SplashPage.html | al., Nature |
| | | | bacterium | | 390:364-370 |
| | | | | | (1997) |
| Bacillus subtilis | 168 | B | Common environmental | http://bioweb.pasteur.fr/GenoLis | Kunst et.al., |
| Genome Size (Mb) 4.20 | | | Organism | t/SubtiList/ | Nature 390: 249- |
| | | | | | 256(1997) |
| Borrelia burgdorferi | B31 | B | Lyme disease | http://www.tigr.org/tdb/CMR/gb | Fraser et al., |
| Genome Size (Mb) 1.44 | | | | b/htmls/SplashPage.html | Nature, 390: |
| | | | | | 580-586 (1997) |
| Chlamydia pneumoniae | CWL029 | B | Acute respiratory infections | http://chlamydia- | Kalman et al., |
| Genome Size (Mb) 1.23 | | | and atherosclerosis. | www.berkeley.edu:4231/ | Nat Genet 21: |
| | | | | | 385-389 (1999) |
| Chlamydia trachomatis | serovar D | B | Obligate pathogen. | http://chlamydia- | Stephens et al., |
| Genome Size (Mb) 1.05 | (D/UW- | | Inclusion conjunctivitis and | www.berkeley.edu:4231/ | Science 282: |
| | 3/Cx) | | genital infections | | 754-759 (1998) |
| Escherichia coil | K-12 | B | Commensal organism, | http://www.genetics.wisc.edu/ | Blattner et. al., |
| Genome Size (Mb) 4.60 | | | Presence of virulence | | Science |
| | | | determinants | | 277:1453-1474 |
| | | | Can cause infections and | | (1997) |
| | | | diarrhoea | | |
| Haemophilus influenzae Rd | KW20 | B | Otitis media, respiratory | http://www.tigr.org/tdb/CMR/gh | Fleischmann et. |
| (non-pathogenic strain) | | | infection and meningitis | i/htmls/SplashPage.html | al., Science |
| Genome Size (Mb) 1.83 | | | | | 269:496-512 |
| | | | | | (1995) |
| Helicobacter pylori | 26695 | B | Chronic active gastritis, | http://www.tigr.org/tdb/CMR/gh | Tomb et. Al., |
| Genome Size (Mb) 1.66 | | | peptic ulceration and | p/htmls/SplashPage.html | Nature 388:539- |
| | | | mucosa-associated | | 547 (1997) |
| | | | lymphoid tissue lymphomas. | | |
| Helicobacter pylori | J99 | B | See H pylori strain 26695 | http://www.astra- | Alm et.al., |
| Genome Size (Mb) 1.64 | | | above | boston.com/hpylori/ | Nature, 397:176- |
| | | | | | 180 (1999) |
| Methanobacterium | delta H | A | Autotrophic methanogenic | http://www.genomecorp.com/se | Smith et al., J. |
| thermoautotrophicum | | | Bacterium | quence center/bacterial genome | Bacteriology, |
| Genome Size (Mb) 1.75 | | | | s/ | 179:7135-7155 |
| | | | | | (1997) |
| Methanococcus jannaschii | DSM 2661 | A | Autotrophic methanogenic | http://www.tigr.org/tdb/CMR/ar | Bult et. al., |
| Genome Size (Mb) 1.66 | | | Bacterium | g/htmls/SplashPage.html | Science |
| | | | | | 273:1058-1073 |
| | | | | | (1996) |
| Mycobacterium | H37Rv | B | Tuberculosis. | http://www.sanger.ac.uk/Project | Cole et al., |
| tuberculosis | (lab strain) | | | s/M tuberculosis/ | Nature 393:537 |
| Genome Size (Mb) 4.40 | | | | | (1998) |
| Mycoplasma genitalium | G-37 | B | Urethritis. | http://www.tigr.org/tdb/CMR/g | Fraser et. Al., |
| Genome Size (Mb) 0.58 | | | | mg/htmls/SplashPage.html | Science |
| | | | | | 270:397-403 |
| | | | | | (1995) |
| Mycoplasma pneumoniae | M129 | B | Respiratory infections | http://www.zmbh.uni- | Himmeireich et. |
| Genome Size (Mb) 0.81 | | | | heidelberg de/M pneumoniae/g | al. Nuc. Acid |
| | | | | enone/Results.html | Res. 24:4420- |
| | | | | | 4449 (1996) |
| Pyrococcus abyssi | GE5 | A | Hyperthermophilic | http://www.genoscope.cns.fr/Pa | |
| Genome Size (Mb) 1.8 | | | Archaebacterium | b/ | |
| Pyrococcus horikoshii | OT3 | A | Hyper-thermophilic | http://www.bio.nite.go.jp/ot3db | Kawarabayasi et |
| Genome Size (Mb) 1.80 | | | Archaebacterium | index.html | al., DNA |
| | | | | | Research 5: 55- |
| | | | | | 76 (1998) |
| Rickettsia prowazekii | Madrid E | B | Epidemic typhus. | http://evolution.bmc.uu.se/˜siv/ | Andersson et al., |
| Genome Size (Mb) 1.10 | | | | gnomics/Rickettsia.html | Nature 396:133- |
| | | | | | 140 (1998) |
| Synechocystis sp. | FCC 6803 | B | Unicellular cyanobacterium | http://www.kazusa.or.jp/cyano/c | Kaneko et. al., |
| Genome Size (Mb) 3.57 | | | | yano.html | DNA Res. 3: |
| | | | | | 109-136 (1996) |
| Thermotoga mantima | MSB8 | B | Thermophile | http://www.tigr.org/tdb/CMR/bt | Nelson et al., |
| Genome Size (Mb) 1.80 | | | | m/htmls/SplashPage.html | Nature 399: 323- |
| | | | | | 329 (1999) |
| Treponema pallidum | Nichols | B | Syphilis | http://www.tigr.org/tdb/CMR/gt | Fraser et al., |
| Genome Size (Mb) 1.14 | | | | p/htmls/SplashPage.html | Science 281: |
| | | | | | 375-388(1998) |

All data correct as of 19/10/1999

TABLE TWO
List of microbial genomes, sequencing in progress (25/10/1999). Sequence data can be
accessed via http://www.ncbi.nlm.nih.gov/BLAST/unfinishedgenome.html#GENOMES

| Genome | Genome | Genome |
|---|---|---|
| Actinobacillus | Klebsiella pneumoniae | Rhodobacter capsulatus |
| actinomycetemcomitans | | |
| Bacillus anthracis | Lactobadilus acidophilus | Rhodobacter sphaeroides |
| Bacillus halodurans | Lactococcus lactis | Rickettsia conorii |
| Bacillus stearothermophilus | Legionella pneumophila | Salmonella paratyphi A |
| Bartonella henselae | Leptospira interrogans | Salmonella typhi |
| | serovar icterohaemorrhagiae | |
| Bordetella bronchiseptica | Listeria monocytogenes | Salmonella typhimurium |
| Bordetella parapertussis | Methanosarcina mazel | Shewanella putrefaciens |
| Bordetella pertussis | Mycobacterium avium | Shigella flexneri 2a |
| Campylobacter jejuni | Mycobacterium bovis | Staphylococcus aureus |
| Caulobacter crescentus | Mycobacterium leprae | Staphylococcus aureus |
| Chlamydia muridarum | Mycobacterium tuberculosis | Streptococcus mutans |
| Chlamydia pneumoniae | Mycoplasma mycoides | Streptococcus pneumoniae |
| | subsp. Mycoides SC | |
| Chlamydia trachomatis | Mycoplasma pulmonis | Streptococcus pyogenes |
| Chlorobium tepidum | Neisseria gonorrhoeae | Streptomyces coelicolor |
| Clostridium acetobutylicum | Neisseria meningitidis | Sulfolobus solfataricus |
| Clostridium diffidile | Neisseria meningitidis | Thermoplasma acidophilum |
| Corynebacterium diphtheriae | Pasteurella haemolytica | Thermus thermophilus |
| Corynebacterium glutamicum | Pasteurella multocida | Thiobacillus ferrooxidans |
| Dehalococcoides | Photorhabdus luminescens | Treponema denticola |
| ethenogenes | | |
| Deinococcus radiodurans | Porphyromonas gingivalis | Ureaplasma urealyticum |
| Desulfovibrio vulgaris | Pseudomonas aeruginosa | Vibrio cholerae |
| Enterococcus faecalis | Pseudomonas putida | Xanthomonas citri |
| Francisella tularensis | Pyrobaculum aerophilum | Xylella fastidiosa |
| Halobacterium salinarium | Pyrococcus furiosus | Yersinia pestis |
| Halobacterium sp. | Raistonia solanacearum | |

TABLE THREE
First Completed Viral Genomes,
taken from GOLD, Genomes On Line Database,
(http://geta.life.uiuc.edu/˜nikos/genomes.html, 25/10/99).

| Viral Genome | Size (Kb) | Publication |
|---|---|---|
| Bacteriophage fX174 | 5.38 | Sanger F et al., 1977 |
| SV40 | 5.224 | Fiers W et al., 1978 |
| Hepatitis B | 3.18 | Galibert F et al., 1979 |
| Bacteriophage Lambda | 48.5 | Sanger F et al.,, 1982 |
| Rous Sarcoma Virus | 9.31 | Schwartz DE et a., 1983 |
| Epstein-Barr Virus | 172.28 | Baer R et al., 1984 |
| AIDS virus LAV | 9.19 | Wain-Hobson S et al., 1985 |
| Vaccinia Virus | 191.63 | Goebel SJ et al., 1990 |
| Cytomegalovirus (CMV) | 229 | Bankier AT et al., 1991 |
| SmallPox Virus (variolla) | 186.102 | Massung RE et al., 1994 |

TABLE FOUR
List of viral genomes, sequencing completed or in progress (from http://www-
fp.mcs.anl.gov/%7Egaasterland/genomes.html, 25/10/99)

| Genome | Genome | Genome | Genome |
|---|---|---|---|
| Abelson murine leukemia | Cucumber green mottle | Jembrana disease virus | Rabies virus |
| virus | mosaic virus | | |
| Adeno-associated virus 2 | Cucumber mosaic virus | Kennedya yellow mosaic virus | Rice tungro spherical |
| | | | virus |
| Adeno-associated virus 3 | Cucumber necrosis virus | Lactate dehydrogenase- | Rice yellow mottle virus |
| | | elevating virus | |
| African swine fever virus | Dengue virus 3 | Leishmania RNA virus | Ross River virus |
| Alfalfa mosaic virus | Dengue virus type 1 | Leishmania RNA virus 1 | Rous sarcoma virus |
| Apple chlorotic leaf spot virus | Dengue virus type 2 | Lucerne transient streak virus | Rubella virus |
| Apple stem grooving virus | Digitaria streak virus | Maize streak virus | Sacoharomyces |
| | | | cerevisiae virus La |
| Arabis mosaic virus satellite | Duck hepatitis B virus | Marburg virus | Saguaro cactus virus |
| Arctic ground squirrel hepatitis | Ebola virus (contructed) | Mason-Pfizer monkey virus | satellite tobacco necrosis |
| B virus | | | virus |
| Artichoke mottled crinkle virus | Eggplant mosaic virus | Measles virus | Sendai virus |
| Autographa californica | Encephalomyocarditis virus | Melon necrotic spot virus | Simian foamy virus |
| nuclear polyhedrosis virus | | | |
| Avian carcinoma virus | Equine infectious anemia | Mice minute virus | Simian immunodeficiency |
| | virus | | virus |
| Avian infectious bronchitis | Feline immunodeficiency virus | Molluscum contagiosum virus | Simian sarcoma virus |
| virus | | subtype 1 | |
| Avian leukosis virus | Foxtail mosaic virus | Moloney murine sarcoma | Simian virus 40 |
| | | virus | |
| Avian sarcoma virus | Friend murine leukemia virus | Mouse mammary tumor virus | Sindbis virus |
| BK virus | Friend spleen focus-forming | Murine leukemia virus | Sindbis-like virus |
| | virus | | |
| Baboon endogenous virus | Fujinami sarcoma virus | Murine osteosarcoma virus | Sonchus yellow net virus |
| Baboon endogenous virus | Ground squirrel hepatitis virus | Murine sarcoma virus | Southern bean mosaic |
| (BaEV) | | | virus |
| Bamboo mosaic virus | Hepatitis A virus | Mushroom bacilliform virus | Soybean chlorotic mottle |
| | | | virus |
| Barley yellow dwarf virus | Hepatitis B virus | Narcissus mosaic virus | Spiroplasma virus |
| Barmah Forest virus | Hepatitis C virus | Onyong-nyong virus | Strawberry vein banding |
| | | | virus |
| Bean golden mosaic virus | Hepatitis D virus | Odontoglossum ringspot virus | Sulfolobus virus-like |
| | | | particle ssv1 |
| Beet curly top virus | Hepatitis E virus | Olive latent virus 1 | Swine vesicular disease |
| | | | virus |
| Beet yellows virus | Hepatitis G virus | Ononis yellow mosaic virus | Theiler's |
| | | | encephalomyelitis virus |
| Black beetle virus | Hepatitis GB virus B | Ovine pulmonary | Tick-borne encephalitis |
| | | adenocarcinoma virus | virus |
| Bombyx mori nuclear | Heron hepatitis B virus | Panicum streak virus | Tobacco etch virus |
| polyhedrosis virus | | | |
| Border disease virus | Hog cholera virus | Papaya mosaic virus | Tobacco mild green |
| | | | mosaic virus |
| Borna disease virus | Human T-cell lymphotropic | Papaya ringspot virus | Tobacco mosaic virus |
| | virus type 1 | | |
| Bovine immunodeficiency | Human T-cell lymphotropic | Pea early browning virus | Tobacco necrosis virus |
| virus | virus type 2 | | |
| Bovine leukemia virus | Human T-cell lymphotropic | Pea seed-borne mosaic virus | Tobacco vein mottling |
| | virus type I (curated proviral) | | virus |
| Bovine viral diarrhea virus | Human adenovirus type 12 | Peanut chlorotic streak virus | Tomato bushy stunt virus |
| Brome mosaic virus | Human adenovirus type 2 | Peanut stripe virus | Tomato golden mosaic |
| | | | virus |
| Cacao swollen shoot virus | Human foamy virus | Peanut stunt virus | Tomato leaf curl virus |
| Caprine arthritis-encephalitis | Human herpesvirus 1 | Pepper huasteco virus | Tomato yellow leaf curl |
| virus | | | virus |
| Cardamine chlorotic fleck | Human herpesvirus 3 | Pepper mottle virus | Turnip vein-clearing virus |
| virus | | | |
| Carrot mottle virus A | Human herpesvirus 4 | Plum pox virus | Turnip yellow mosaic |
| | | | virus |
| Cassava common mosaic | Human immunodeficiency | Polyomavirus strain a2 | Vaccinia virus |
| virus | virus type 1 | | |
| Cassava latent virus | Human immunodeficiency | Polyomavirus strain a3 | Variola virus |
| | virus type 2 | | |
| Cassava vein mosaic virus | Human parainfluenza virus 3 | Potato leaf roll virus | Venezuelan equine |
| | | | encephalitis virus |
| Cauliflower mosaic virus | Human respiratory syncytial | Potato mop-top virus | Vesicular stomatitis virus |
| | virus | | |
| Chicken anemia virus | Infectious hematopoietic | Potato virus A | Visna virus |
| | necrosis virus | | |
| Chloris striate mosaic virus | Influenza A virus | Potato virus M | West Nile virus |
| Citrus tristeza virus | Influenza B virus | Potato virus X | Woodchuck hepatitis B |
| | | | virus |
| Clover yellow mosaic virus | Influenza C virus | Potato virus Y | Woodchuck hepatitis |
| | | | virus |
| Coconut foliar decay virus | JC virus | Punta Toro virus | Y73 sarcoma virus |
| Commelina yellow mottle | Japanese encephalitis virus | Rabbit hemorrhagic disease | Yellow fever virus |
| virus | | virus | |

TABLE FIVE
List of eukaryotic pathogens, sequencing in progress, taken from GOLD, Genomes On
Line Database, (http://geta.life.uiuc.edu/˜nikos/genomes.html, 25/10/99).
1. Protozoa

| Genome | Strain | Disease/Description | Web site | Publication |
|---|---|---|---|---|
| Leishmania major | Friedlin | Cutaneous leishmaniasis | http://www.sbri.org/Labs/myler.html, | Chromosome |
| | | | http://www.sanger.ac.uk/Projects/L_major/ | complete |
| | | | beowulf_index.shtml | Myler et al., |
| | | | http://204.203.14.2/LmjF/chr3.html | Natl Acad Science |
| | | | http://204.203.14.2/LmjF/chr35.html | 96: 2902-29 |
| | | | | (1999) |
| Plasmodium falciparum | 3D3 | Human malaria | http://www.tigr.org/tdb/edb/pfdb/pfdb.html | Chromosome |
| | | | http://www.ncbi.nlm.nih.gov/Malaria/ | 3 complete |
| | | | http://www.sanger.ac.uk/Projects/ | 282,1126-1 |
| | | | P_falciparum/ | (1998) |
| Cryptosporidium parvum | | Diarrhoea | http://www.mrc- | |
| | | | lmb.cam.ac.uk/happy/CRYPTO/crypto- | |
| | | | genome.html | |
| Dictyostelium | AX4 | A soil-living amoeba | http://www.sanger.ac.uk/Projects/D_discoideu | |
| discoideum | | | m/ | |
| Giardia lamblia | WB | Diarrhoea | http://evol3.mbl.edu/Giardia- | |
| | | | HTML/giardia_data.html | |
| Shistosoma mansoni | | Shistosomiasis | http://www.nhm.ac.uk/hosted_sites/schisto/ind | |
| | | | ex.html | |
| Shistosoma japonicum | | Shistosomiasis | http://www.nhm.ac.uk/hosted_sites/schisto/ind | |
| | | | ex.html | |
| Trypanosoma brucei | | Trypanosomiasis | http://www.sanger.ac.uk/Projects/T_brucei/ | |
| | | (West African sleeping | | |
| | | sickness), | | |
| Trypanosoma b. | TREU | Rhodesian | http://www.tigr.org/tdb/mdb/tbdb/ | |
| rhodesiense | 927/4 | trypanosomiasis | http://www.tigr.org/cgi- | |
| | | | in/BlastSearch/blast.cgi?organism=t_brucei | |
| Trypanosoma cruzi | | Chagas' disease | http://www.tigr.org/ | |

2. Microsporidia

| Genome | Disease/Description | Web site |
|---|---|---|
| Encephalitozoon cuniculi | Intracellular parasite | http://www.genoscope.cns.fr/externe/English/Projets/Projet_ |
| | | AD/AD.html |

3. Fungi

| Genome | Disease/Description | Web site |
|---|---|---|
| Schizosaccharomyces | Fission yeast | http://genome- |
| pombe | | www.stanford.edu/Saccharomyces/VL-yeast.html |
| | | and |
| | | http//www.sanger.ac.uk/Projects/S_pombe/ |
| Neurospora crassa | A genus of fungi used as a | http://www.genome.ou.edu/fungal.html |
| strain 74-OR23-IVA | model organism in genetic research | Neurospora Genome Project |
| | | http://biology.unm.edu/˜ngp/home.html |
| Aspergillus ridulans | Mycelial fungus, may cause aspergillosis | http://fungus.genetics.uga.edu:5080/ |
| Candida albicans 1161 | Common human pathogen | http://www.sanger.ac.uk/Projects/C_albicans/ |
| Candida albicans | | http://sequence- |
| SC5314 | | www.stanford.edu/group/candida/index.html |
| Pneumocystis carinii | Extracellular lung pathogens that can | http://www.uky.edu/Projects/Pneumocystis/ |
| f. sp. Carinii | lead | |
| | to development of a lethal pneumonia. | |
| Pneumocystis carinii | | http//www.uky.edu/Projects/Pneumocystis/ |
| f. sp. Hominis | | |
| Ustilago maydis | ‘Corn smut’, allergen. | http://www3.ncbi.nlm.nih.gov/htbin- |
| | | post/Taxonomy/wgetorg?id=5270&1v1=3 |

### EXAMPLE 1

### SOME BACTERIAL AND VIRAL PATHOGENS

Haemophilus influenzae

H. influenzae was the first free-living unicellular organism to be completely sequenced in 1995. It is a small, nonmotile, Gram-negative bacterium whose only natural host is human.

These bacteria were first identified during the influenza (‘flu’) pandemic of 1890. At the time it was believed to be the cause of the disease which is now known to be viral in origin. It is an obligate parasite, having an absolute requirement for exogenously supplied heme for aerobic growth. There are six antigenically distinct capsular types of H. infiuenzae, designated a to f. Non-typeable strains also exist and are distinguished by their lack of detectable capsular polysaccharide. They are frequent constituents of the upper respiratory mucosa of healthy children and adults. Serious invasive infection is caused almost exclusively by type b strains; these include meningitis, sepsis, epiglottitis, pneumonia and inner-ear infections.

Bacterial meningitis and epiglottitis due to H. infiuenzae are lfe-threateming diseases with a 5-25% lethality. These statistics make the study of H. influenzae a very important area of medical research.

The H. influenzae bacillus is also exhibiting increased antibiotic resistance. The first finding of ampicillin resistance dates to 1984. As a result, current pharmacological research is focusing on the development of antibiotics that specifically target this microorganism. There is also a clear association between infection by H. influenzae and infection by the human immunodeficiency virus (HIV).

The strain from which the complete genome sequence has been determined is the non-pathogenic H. influenzae Rd strain KW20. The only difference between noninfectious Rd and infectious type b strains of H. influenzae is the presence in type b of a set of eight, tandemly arrayed genes that encode fimbrial proteins. Fimbriae are colonization factors that mediate bacterial adherence to human cells. These genes have also been screened for complementary peptides.

The sequencing of the H. influenzae chromosome is a very important landmark in biological research since this is the first complete genome sequence of a free-living organism (Fleischmann et al., 1995). The circular chromosome of this microorganism is 1.83 Mb long, with an overall G+C content of approximately 38%. The authors identified 1743 open reading frames (ORFs) in the sequence. Sixty-three of these ORFs contain frameshifts or stop codons when compared to homologues from other species. A total of 1,007 genes have been matched to the biological database; 347 matched hypothetical proteins already in the database, and 389 did not have any matches.

Recently, the H. influenzae genome sequence has been re-analyzed, resulting in a new set of predicted genes among ORFs without homologs (Tatusov et al., 1996). The H. influenzae sequences found in GenBank have been re-annotated based on this analysis.

Borrelia burgdorferi

The genus Borrelia is one of the four genera of the family Spirochaetaceae and comprises pathogenic bacteria that are transmitted by arthropod vectors. Borrelia species utilize glucose as the major energy source, and lactic acid is the predominant metabolic end product.

B. burgdorferi is the causative agent of Lyme borreliosis, a disease transmitted by ticks. The disease is named after the town Old Lyme, Connecticut, USA, where a mysterious cluster of arthritis cases occurred among children in the early 1970s. The illness was recognized as a distinctive disease and called Lyme disease. The most common symptoms of Lyme disease are rash, muscle and joint aches, headache and stiff neck, fatigue, facial paralysis, and meningitis. During more advanced stages of the disease, infected individuals experience arthritis, intermittent or chronic. Lyme disease is difficult to diagnose because many of its symptoms mimic those of other disorders. Almost all Lyme disease patients can be effectively treated with antibiotic therapy, such as doxycycline or amoxicillin.

The DNA organization of the Borrelia appears to be unique in the spirochete family in having linear DNA plasmids, a form of DNA that was previously thought to be unique to eukaryotes. Borrelia species also contain circular plasmids. The entire genome of B. burgdorferi is 1.0 Mb in length and was completely sequenced in 1997 (Fraser et al., 1997).

Hepatitis B

Hepatitis B is the second most common chronic infectious disease worldwide. When adults are infected, about 90% of them are able to defeat the hepatitis B virus on its own but in 10 % of cases, the disease wins out over the immune system and the condition becomes chronic. Individuals who suffer from chronic hepatitis B are at high risk of developing cirrhosis of the liver and liver cancer. According to the World Health Report published by the World Health Organization in 1997, 2 billion people have evidence of past or current infection with the !I hepatitis B virus, and 350 million are chronically infected.

In Western countries, Hepatitis B virus is transmitted prinicpally via blood products, intravenous drug use, or sexually. In other parts of the world, particularly in Asia, the major route of transmission is from infected mother to child at birth. Children are particularly susceptible to the virus; as many as 50-70% of those exposed to the virus become chronic carriers.

Hepatitis C

Hepatitis C is one of the world's most prevalent chronic infectious diseases. In approximately 85 % of all cases, the body is not able to fight off the infection and the infected individual becomes a chronic hepatitis C carrier. The World Health Organization estimates that more than 170 million people are infected worldwide with the hepatitis C virus.

The hepatitis C virus was not specifically identified until 1989. Approximately 20% of infected persons develop cirrhosis of the liver within 10-20 years after infection. For others, the rate of disease progression is much slower and may extend over 20 to 40 years or more

Aside from the fact that it is a blood borne disease, the mechanism of spread of hepatitis C within a population is poorly understood. Hepatitis C virus is transmitted via blood products, intravenous drug use, and sexually but, in some cases, its origin remains unknown. Healthcare workers are particularly susceptible to hepatitis C infection.

### EXAMPLE 2

The complete genome of Mycoplasma genitalium which is 0.58 Mb in size and codes for an estimated 479 genes was screened for intermolecular peptides using the method described in patent application number GB 9927485.4, filed 19th November 1999. The gene, database accession number, its predicted interacting peptides and their position within the coding sequence of the gene are shown in the attached sequence listing: SEQ ID Nos. [1-754].

### EXAMPLE 3

Derivation of ‘Child’ Sequences from Parent Sequences

For each pair of ‘frames’ of amino acids which are deemed a ‘hit’ by the algorithm of the current invention includes derived pairs of composite ‘child’ sequences of shorter frame lengths which automatically fulfil the same ‘complementary’ relationship.

For example, there is a complementary frame of size 10 between genes (inter-molecular) MG002 and MG004 of mycoplasma genitalium.:

| GENE1 | GENE2 | Sequence 1 | Location | Sequence 2 | Location | Score |
|---|---|---|---|---|---|---|
| MG002 | MG004 | AYSILSDPNQ | 47-56 | LLSVGQNGIG | 720-729 | 10 |

One embodiment of the invention covers the derivation of the following sequences at frame length of 5:

| | | Se- | | | | |
|---|---|---|---|---|---|---|
| | | quence | | | | |
| GENE | GENE2 | 1 | Location | Sequence 2 | Location | Score |
| MG002 | MG004 | AYSIL | 47-51 | LLSVG | 720-724 | 5 |
| MG002 | MG004 | YSILS | 48-52 | LSVGQ | 721-725 | 5 |
| MG002 | MG004 | SILSD | 49-53 | SVGQN | 722-726 | 5 |
| MG002 | MG004 | ILSDP | 50-54 | VGQNG | 723-727 | 5 |
| MG002 | MG004 | LSDPN | 51-55 | GQNGI | 724-728 | 5 |
| MG002 | MG004 | SDPNQ | 52-56 | QNGIG | 725-729 | 5 |

One embodiment of the invention covers the derivation of the following sequences at frame length of 6:

| | | Sequence | | Sequence | | |
|---|---|---|---|---|---|---|
| GENE | GENE2 | 1 | Location | 2 | Location | Score |
| MG002 | MG004 | AYSILS | 47-52 | LLSVGQ | 720-725 | 6 |
| MG002 | MG004 | YSILSD | 48-53 | LSVGQN | 721-726 | 6 |
| MG002 | MG004 | SILSDP | 49-54 | SVGQNG | 722-727 | 6 |
| MG002 | MG004 | ILSDPN | 50-55 | VGQNGI | 723-728 | 6 |
| MG002 | MG004 | LSDPNQ | 51-56 | GQNGIG | 724-729 | 6 |

One embodiment of the invention covers the derivation of the following sequences at frame length of 7:

| | | Sequence | Loca- | | | |
|---|---|---|---|---|---|---|
| GENE | GENE2 | 1 | tion | Sequence 2 | Location | Score |
| MG002 | MG004 | AYSILSD | 47-53 | LLSVGQN | 720-726 | 7 |
| MG002 | MG004 | YSILSDP | 48-54 | LSVGQNG | 721-727 | 7 |
| MG002 | MG004 | SILSDPN | 49-55 | SVGQNGI | 722-728 | 7 |
| MG002 | MG004 | ILSDPNQ | 50-56 | VGQNGIG | 723-729 | 7 |

One embodiment of the invention covers the derivation of the following sequences at frame length of 8:

| | | | Loca- | | Loca- | |
|---|---|---|---|---|---|---|
| GENE | GENE2 | Sequence 1 | tion | Sequence 2 | tion | Score |
| MG002 | MG004 | AYSILSDP | 47-54 | LLSVGQNG | 720- | 8 |
| | | | | | 727 | |
| MG002 | MG004 | YSILSDPN | 48-55 | LSVGQNGI | 721- | 8 |
| | | | | | 728 | |
| MG002 | MG004 | SILSDPNQ | 49-56 | SVGQNGIG | 722- | 8 |
| | | | | | 729 | |

One embodiment of the invention covers the derivation of the following sequences at frame length of 9:

| GENE | GENE2 | Sequence 1 | Location | Sequence 2 | Location | Score |
|---|---|---|---|---|---|---|
| MG002 | MG004 | AYSILSDPN | 47-55 | LLSVGQNGI | 720-728 | 9 |
| MG002 | MG004 | YSILSDPNQ | 48-56 | LSVGQNGIG | 721-729 | 9 |

### EXAMPLE 4

The complete genome of Mycoplasma genitalium which is 0.58 Mb in size and codes for

an estimated 479 genes was screened for intramolecular peptides using the method described in patent application number GB 9927485.4, filed 19th Nov. 1999. The gene, database accession number, peptide sequences and their position within the coding sequence of the gene are shown in the attached sequence listing: SEQ ID Nos. [755-804].

### EXAMPLE5

Derivation of ‘Child’ Sequences from Parent Sequences

For each pair of ‘framnes’ of amino acids which are deemed a ‘hit’ by the algorithm of the current invention includes derived pairs of composite ‘child’ sequences of shorter frame lengths which automatically fuilfil the same ‘complementary’ relationship.

For example, gene MG015 in Mycoplasma Genitalium contains the following intra-molecular complementary relationship of frame length 10:

| GENE | Sequence 1 | Location | Sequence 2 | Location | Score |
|---|---|---|---|---|---|
| MG015 | SFAFLKKSKT | 184-193 | SFAFLKKSKT | 184-193 | 10 |

One embodiment of the invention covers the derivation of the following sequences at frame lengthof 5:

| GENE | Sequence 1 | Location | Sequence 2 | Location | Score |
|---|---|---|---|---|---|
| 1787318 | SFAFL | 184-188 | TKSKK | 193-189 | 5 |
| 1787318 | FAFLK | 185-189 | KSKKL | 192-188 | 5 |
| 1787318 | AFLKK | 186-190 | SKKLF | 191-187 | 5 |
| 1787318 | FLKKS | 187-191 | KKLFA | 190-186 | 5 |
| 1787318 | LKKSK | 188-192 | KLFAF | 189-185 | 5 |
| 1787318 | KKSKT | 189-193 | LFAFS | 188-184 | 5 |

One embodiment of the invention covers the derivation of the following sequences at of 6:

| GENE | Sequence 1 | Location | Sequence 2 | Location | Score |
|---|---|---|---|---|---|
| 1787318 | SFAFLK | 184-189 | TKSKKL | 193-188 | 6 |
| 1787318 | FAFLKK | 185-190 | KSKKLF | 192-187 | 6 |
| 1787318 | AFLKKS | 186-191 | SKKLFA | 191-186 | 6 |
| 1787318 | FLKKSK | 187-192 | KKLFAF | 190-185 | 6 |
| 1787318 | LKKSKT | 188-193 | KLFAFS | 189-184 | 6 |

One embodiment of the invention covers the derivation of the following sequences at of 7:

| GENE | Sequence 1 | Location | Sequence 2 | Location | Score |
|---|---|---|---|---|---|
| 1787318 | SFAFLKK | 184-190 | TKSKKLF | 193-187 | 7 |
| 1787318 | FAFLKKS | 185-191 | KSKKLFA | 192-186 | 7 |
| 1787318 | AFLKKSK | 186-192 | SKKLFAF | 191-185 | 7 |
| 1787318 | FLKKSKT | 187-193 | KKLFAFS | 190-184 | 7 |

One embodiment of the invention covers the derivation of the following sequences at of 8:

| GENE | Sequence 1 | Location | Sequence 2 | Location | Score |
|---|---|---|---|---|---|
| 1787318 | SFAFLKKS | 184-191 | TKSKKLFA | 193-186 | 8 |
| 1787318 | FAFLKKSK | 185-192 | KSKKLFAF | 192-185 | 8 |
| 1787318 | AFLKKSKT | 186-193 | SKKLFAFS | 191-184 | 8 |

| | | | | Loca- | |
|---|---|---|---|---|---|
| GENE | Sequence 1 | Location | Sequence 2 | tion | Score |
| 1787318 | SFAFLKKSK | 184-192 | TKSKKLFAF | 193- | 9 |
| | | | | 185 | |
| 1787318 | FAFLKKSKT | 185-193 | KSKKLFAFS | 192- | 9 |
| | | | | 184 | |

One embodiment of the invention covers the derivation of the following sequences at frame length of 9:

### EXAMPLE 6

The genomes of the following microbes were screened for intermolecular peptides in the same way as in Example 2.

| Genome | Number of proteins |
|---|---|
| Borrelia burgdorferi | 849 |
| Chlamydia Pneumoniae | 1051 |
| Chlamydia Trachomatis | 893 |
| Echerichia Coli | 4288 |
| Haemophilus Influenzae | 1708 |
| Helicobacter Pylori | 1552 |
| Mycobacterium tuberculosis | 3924 |
| Mycoplasma genitalium | 479 |
| Mycoplasma pneumoniae | 676 |
| Rickettsia Prowazekii | 833 |
| Treponema Pallidum | 1030 |

### EXAMPLE 7

The genomes of the following microbes were screened for intramolecular peptides in the same way as in Example 4.

| Genome | Number of proteins |
|---|---|
| Borrelia burgdorferi | 849 |
| Chlamydia Pneumoniae | 1051 |
| Chlamydia Trachomatis | 893 |
| Echerichia Coli | 4288 |
| Haemophulus Influenzae | 1708 |
| Helicobacter Pylori | 1552 |
| Mycobacterium tuberculosis | 3924 |
| Mycoplasma genitalium | 479 |
| Mycoplasma pneumoniae | 676 |
| Rickettsia Prowazekii | 833 |
| Treponema Pallidum | 1030 |

### EXAMPLE 8

THE AMINO ACID PAIRINGS RESULTING FROM READING THE ANTICODON FOR
NATURALLY OCCURING AMINO ACID RESIDUES IN THE 5′-3′ DIRECTION

| | | Comple- | | | | Comple- | Comple- |
|---|---|---|---|---|---|---|---|
| Amino | | mentary | Complementary | Amino | | mentary | mentary |
| Acid | codon | codon | Amino acid | Acid | codon | codon | Amino acid |
| Alanine | GCA | UGC | Cysteine | Serine | UCA | UGA | Stop |
| | GCG | CGC | Arginine | | UCC | GGA | Glycine |
| | GCC | GGC | Glycine | | UCG | CGA | Arginine |
| | GGU | AGC | Serine | | UCU | AGA | Arginine |
| | | | | | AGC | GCU | Alanine |
| | | | | | AGU | AGU | Threonine |
| Arginine | CGG | CCG | Proline | Glutamine | CAA | UUG | Leucine |
| | CGA | UCG | Serine | | CAG | CUG | LeucINe |
| | CGC | GCG | Alanine | | | | |
| | CGU | ACG | Threonine | | | | |
| | AGG | CCU | Proline | | | | |
| | AGA | UCU | Serine | | | | |
| Aspartic Acid | GAC | GUC | Valine | Glycine | GGA | UCC | Serine |
| | GAU | AUC | Isoleucine | | GGC | GCC | Alanine |
| | | | | | GGU | ACC | Threonine |
| | | | | | GGG | CCC | Proline |
| Asparagine | AAC | GUU | Valine | Histidine | CAC | GUG | Valine |
| | AAU | AUU | Isoleucine | | CAU | AUG | Methionine |
| Cysteine | UGU | AGA | Threonine | Isoleucine | AUA | UAU | Tyrosine |
| | UGC | GCA | Alanine | | AUC | GAU | Aspartic |
| | | | | | AUU | AAU | acid |
| | | | | | | | Asparagine |
| Glutamic | GAA | UUC | Phenylalanine | Leucine | CUG | CAG | Glutamine |
| Acid | GAG | CUC | Leucine | | CUC | GAG | Glutamic |
| | | | | | CUU | AAG | acid |
| | | | | | UUA | UAA | Lysine |
| | | | | | CUA | UAG | Stop |
| | | | | | UUG | CAA | Stop |
| | | | | | CUG | CAG | Glutamine |
| | | | | | | | Glutamine |
| Lysine | AAA | UUU | Phenylalanine | Threonine | ACA | UGU | Cysteine |
| | AAG | CUU | Leucine | | ACG | CGU | Arginine |
| | | | | | ACC | GGU | Glycine |
| | | | | | ACU | AGU | Serine |
| Methionine | AUG | CAU | Histidine | Tryptophan | UGG | CCA | Proline |
| Phenylalanine | UUU | AAA | Lysine | Tyrosine | UAC | GUA | Valine |
| | UUC | GAA | Glutamic Acid | | UAU | AUA | Isoleucine |
| Proline | GCA | UGG | Tryptophan | Valine | GUA | UAC | Tyrosine |
| | CCC | GGG | Glycine | | GUG | CAC | Histidine |
| | CCU | AGG | Arginine | | GUC | GAC | Aspartic |
| | CCG | CGG | Arginine | | GUU | AAC | Acid |
| | | | | | | | Asparagine |

### EXAMPLE 9

The relationships between amino acids and the residues encoded in the complementary strand reading 3‘-5’

| | | Comple- | | | | Comple- | Comple- |
|---|---|---|---|---|---|---|---|
| Amino | | mentary | Complementary | Amino | | mentary | mentary |
| Acid | codon | codon | Amino acid | Acid | codon | codon | Amino acid |
| Alanine | GCA | CGU | Arginine | Serine | UCA | AGU | Serine |
| | GCG | CGC | | | UCC | AGG | Arginine |
| | GCC | CGG | | | UCG | AGC | Serine |
| | GCU | CGA | | | UCU | AGA | Arginine |
| | | | | | AGC | UCG | Serine |
| | | | | | AGU | UCA | Serine |
| Arginine | CGG | GCC | Alanine | Glutamine | CAA | GUU | Valine |
| | CGA | GCU | Alanine | | CAG | GUC | Valine |
| | CGC | GCG | Alanine | | | | |
| | CGU | GCA | Alanine | | | | |
| | AGG | UCC | Serine | | | | |
| | AGA | UCU | Serine | | | | |
| Aspartic | GAC | GUC | Valine | Glycine | GGA | CCU | Proline |
| Acid | GAU | AUC | Isoleucine | | GGC | CCG | Proline |
| | | | | | GGU | CCA | Proline |
| | | | | | GGG | CCC | Proline |
| Asparagine | AAC | UUG | Leucine | Histidine | CAC | GUG | Valine |
| | AAU | UUA | Leucine | | CAU | GUA | Valine |
| Cysteine | UGU | ACA | Threonine | Isoleucine | AUA | UAU | Tyrosine |
| | UGC | ACG | Threonine | | AUC | UAG | Stop |
| | | | | | AUU | UAA | Stop |
| Glutamic | GAA | CUU | Leucine | Leucine | CUG | GAC | Asp |
| Acid | GAG | CUG | Leucine | | CUC | GAG | Glutamic |
| | | | | | CUU | GAA | acid |
| | | | | | UUA | AAU | Glutamic |
| | | | | | CUA | GAU | Acid |
| | | | | | UUG | AAC | Asparagine |
| | | | | | CUG | GAC | Aspartic |
| | | | | | | | Acid |
| | | | | | | | Asparagine |
| | | | | | | | Aspartic |
| | | | | | | | Acid |
| Lysine | AAA | UUU | Phenylalanine | Threonine | ACA | UGU | Cysteine |
| | AAG | UUC | Phenylalanine | | ACG | UGC | Cysteine |
| | | | | | ACC | UGG | Tryptophan |
| | | | | | ACU | UGA | Stop |
| Methionine | AUG | UAC | Tyrosine | Tryptophan | UGG | ACC | Threonine |
| Phenylalanine | UUU | AAA | Lysine | Tyrosine | UAC | AUG | Methionine |
| | UUC | AAG | Lysine | | UAU | AUA | Isoleucine |
| Proline | CCA | GGU | Glycine | Valine | GUA | CAU | Histidine |
| | CCC | GGG | Glycine | | GUG | CAC | Histidine |
| | CCU | GGA | Glycine | | GUC | CAG | Glutamine |
| | CCG | GGC | Glycine | | GUU | CAA | Glutamine |

### REFERENCES

All publications, patents, and patent applications cited are hereby incorporated by reference in their entirety.

Baranyi L, Campbell W, Ohshima K, Fujimoto S, Boros M and Okada H. 1995. The antisense homology box: a new motif within proteins that encodes biologically active peptides. NatureMedicine. 1:894-901.

Fleischmann RD, Adams MD, White 0, Clayton RA, Kirkness EF, Kerlavage AR, Bult CJ, Tomb JF, Dougherty BA, Merrick JM, et al. 1995. Whole-genome random sequencing and assembly of Haemophilus influenzae Rd. Science 269:496-512.

Fraser CM, Casjens S, Huang WM, Sutton GG, Clayton R, Lathigra R, White O, Ketchum KA, Dodson R, Hickey EK, Gwinn M, Dougherty B, Tomb JF, Fleischmann RD, Richardson D, Peterson J, Kerlavage AR, Quackenbush J, Salzberg S, Hanson M, van Vugt R, Palmer N, Adams MD, Gocayne J, Venter JC, et al. 1997. Genomic sequence of a Lyme disease is spirochaete, Borrelia burgdorferi. Nature 390:580-6.

Gaasterland T. Structural genomics: Bioinformatics in the driver's seat. Nature Biotechnology 16: 645-627, 1998.

Goldstein DJ. 1998. An unacknowledged problem for structural genomics? Nature Biotechnology 16: 696-697.

Sansom C. 1998. Extending the boundaries of molecular modelling. Nature Biotechnology 16: 917-918.

Stryer L. Biochmistry. 4th Edition. Freeman and Company, New York 1997.

Tatusov RL, Mushegian AR, Bork P, Brown NP, Hayes WS, Borodovsky M, Rudd KE and Koonin EV. 1996. Metabolism and evolution of Haemophilus influenzae deduced from a whole-genome comparison with Escherichia coli. Curr Biol. 6:279-91.

We claim:

1. A set of peptide ligands; said set consisting of specific complementary peptides to proteins encoded by genes of the genome of a microbe.

2. A set of peptide ligands according to claim 1, wherein the genome is of a pathogenic microbe.

3. A set of peptide ligands according to claim 2, wherein the pathogenic microbe is selected from the group consisting of Borrelia burgdorferi, Chlamydia Pneumoniae, Chlamydia Trachomatis, Echerichia Coli, Haemophilus Influenzae, Helicobacter Pylori, Mycobacterium tuberculosis, Mycoplasma genitalium, Mycoplasma pneumoniae, Rickettsia Prowazekii and Treponema Pallidum.

4. A set of peptide ligands according to claim 3, wherein the sequences of the peptides in the set are intra-molecular complementary peptide sequences and are selected from the group consisting of Borrelia burgdorferi, Chlamydia Pneumoniae, Chlamydia Trachomatis, Echerichia Coli, Haemophilus Influenzae, Helicobacter Pylori, Mycobacterium tuberculosis, Mycoplasma genitalium, Mycoplasma pneumoniae, Rickettsia Prowazekii and Treponema Pallidum.

5. A set of peptide ligands according to claim 3, wherein the sequences of the peptides in the set are inter-molecular complementary peptide sequences and are selected from the group consisting of Borrelia burgdorferi, Chlamydia Pneumoniae, Chlamydia Trachomatis, Echerichia Coli, Haemophilus Influenzae, Helicobacter Pylori, Mycobacterium tuberculosis, Mycoplasma genitalium, Mycoplasma pneumoniae, Rickettsia Prowazekii and Treponema Pallidum.

6. A novel peptide having a sequence which is a member of a set according to any preceding claim, capable of antagonising or agonising a specific interaction of a protein with another protein or receptor.

7. Use of a set of peptides according to any of claims 1 to 5 in an assay for screening and identification of one or more peptides according to claim 6.

8. Use according to claim 7 wherein the identified peptide(s) is an anti-infective drug candidate.

9. Use according to claim 7 wherein the identified peptide(s) is an anti-infective pro-drug.

10. A partly or wholly non-peptide mimetic of a peptide drug candidate or pro-dlrug according to claim 6, 8 or 9, identified by use of the set of peptides a ccording to claim 7.

11. A method for identifying a peptide drug candidate or pro-drug which is anti-infective against a microbe, which method includes the steps of (i) identifying a set of specific complementary peptides according to any of claims 1 to 5; (ii) screening the set for specific protein interaction activity; and (iii) identifying one or more peptide(s) according to claim 6.

12. A method for processing sequence data comprising the steps of;
selecting a first protein sequence and a second protein sequence;
selecting a frame size corresponding to a number of sequence elements such as amino acids or triplet codons, a score threshold, and a frame existence probability threshold;
comparing each frame of the first sequence with each frame of the second sequence by comparing pairs of sequence elements at corresponding positions within each such pair of frames to evaluate a complementary relationship score for each pair of frames;
storing details of any pairs of frames for which the score equals or exceeds the score threshold;
evaluating for each stored pair of frames the probability of the existence of that complementary pair of frames existing, on the basis of the number of possible complementary sequence elements existing for each sequence element in the pair of frames; and
discarding any stored pairs of frames for which the evaluated probability is greater than the probability threshold; wherein each frame is a peptide sequence of defined length.

13. A method according to claim 12, in which the first sequence is identical to the second sequence and a frame at a given position in the first sequence is only compared with frames in the second sequence at the same given position or at later positions in the second sequence, in order to eliminate repetition of comparisons.

14. A method according to claim 12 or 13, in which the sequence elements at corresponding positions within each of a pair of frames are compared sequentially, each such pair of sequence elements generating a score which is added to an aggregate score for the pair of frames.

15. A method according to claim 14, in which if the aggregate score reaches the score threshold before all the pairs of sequence elements in the pair of frames have been compared, details of the pair of frames are immediately stored and a new pair of frames is selected for comparison.

16. A method according to any preceding claim, in which the sequence elements are amino acids and pairs of amino acids are compared by using an antisense score list.

17. A method according to any of claims 12 to 15, in which the sequence elements are triplet codons and pairs of codons in corresponding positions within each of the pairs of triplet codons are compared by using an antisense score list.

18. A method for processing sequence data substantially as described herein with reference to FIGS. 1 to 6.

19. A pair of frames or a list of pairs of frames being the product of the method of any of claims 12 to 18, optionally carried on a computer-readable medium.

20. A frame being the product of the method of any of claims 12 to 18, optionally carried on a computer-readable medium.

21. A peptide, pair of complementary peptides, or set of peptides, being the peptide(s) having the sequence of the frame(s) of claim 19 or 20.
